(19) United States
(12) Patent Application Publication
(10) Pub. No.: US 2004/0013687 A1
(43) Pub. Date: Jan. 22, 2004

(54) Compositions and methods for transepithelial molecular transport

(75) Inventors: Lance Simpson, Moorestown, NJ (US); Andrew Maksymowych, Gulph Mills, PA (US); Jung-Beak Park, Philadelphia, PA (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(73) Assignee: Thomas Jefferson University

(21) Appl. No.: 10/452,024

(22) Filed: Jun. 2, 2003

Related U.S. Application Data
(60) Provisional application No. 60/384,949, filed on May 31, 2002.

(51) Int. Cl. A61K 39/02

(52) U.S. Cl. 424/190.1

(57) ABSTRACT

The invention relates to fragments of Clostridium botulinum HC that can be linked with an entity (e.g., an antigen, a particle, or a radionuclide) and used to deliver the entity across a non-keratinized epithelial membrane of an animal. The fragments are useful, for example, for making vaccines, antidotes, and anti-toxins and in situations in which rapid uptake of an agent by an animal is desired.

### CROSS-REFERENCE TO RELATED APPLICATIONS

This application claims priority under 35 U.S.C. § 119(e) to U.S. provisional patent application serial No. 60/384,949, filed May 31, 2002, the contents of which are incorporated herein by reference.

### STATEMENT REGARDING FEDERALLY SPONSORED RESEARCH OR DEVELOPMENT

This research was supported in part by U.S. Government funds (National Institutes of Health grant number GM057342).

### BACKGROUND OF THE INVENTION

Botulinum toxin is the causative agent of botulism and other disorders in humans and other animals (e.g., other mammals, reptiles, birds, and amphibians). The pathological effects of this agent are mediated by a neurotoxin that is able to cross the gut and airway epithelium to enter the general circulation. Once in the circulation, botulinum neurotoxin (“BoNT”) is able to bind to the presynaptic membrane of neuromuscular junctions and thereafter enter the neuronal cytosol. In the cytosol, BoNT blocks neuronal release of acetylcholine at the neuromuscular junction, causing flaccid paralysis of the muscle.

BoNT is synthesized as a single-chain inactive propolypeptide having a molecular mass of approximately 150 kilodaltons. Inactive pro-BoNT is activated by proteolytic cleavage of the pro-BoNT by endogenous or exogenous proteases. Cleavage (“nicking”) of the inactive BoNT propeptide yields two polypeptide chains, a heavy chain (“HC”) and a light chain (“LC”). The HC and LC normally remain linked by a disulfide bond that can be severed under reducing conditions, such as those that exist in the interior of an animal cell.

Several species of Clostridia are presently known to produce the BoNT toxin, including Clostridium botulinum, Clostridium baratii, and Clostridium butyricum.

BoNT is presently known to be produced in seven immunologically distinct forms, A, B, C, D, E, F, and G. In nature, each serotype is released from clostridia in association with two classes of proteins (sometimes referred to as the “auxiliary proteins”): (i) a family of hemagglutinins (“HA”) and (ii) a single, nontoxin, non-hemagglutinin protein (“NTNH”).

BoNT is known to be able to penetrate gut, pulmonary, and other epithelial membranes in order to gain access to the bloodstream. In the bloodstream, BoNT is able to enter neurons at the neuromuscular junction, whereupon the toxin can manifest its characteristic effects.

The ability of BoNT molecules altered such that some or all of the LC has been deleted to cross epithelial membranes has been described (e.g., U.S. Pat. No. 6,051,239). However, those molecules require production or isolation of intact HC, which has proven impractical for various reasons. Others have attempted to prepare injectable vaccines for preventing botulism using fragments of BoNTs. However, none describes vaccines that prevent botulism which are capable of transcytosing across epithelia.

A need remains for improved compositions and methods for delivering antigens, drugs, imaging agents, radionuclides, and other agents to the bodies of animals via the epithelium. The invention satisfies this need, at least in part, by providing compositions and methods for delivering entities across animal epithelial membranes.

### BRIEF SUMMARY OF THE INVENTION

The invention is based on the discovery that carboxyterminal fragments of the HC of Clostridium botulinum neurotoxin (BoNT) have the ability to cross epithelial membranes in animals. A surprising further discovery is that fragments of the HC of BoNT are able to mediate transepithelial transport of a wide range of entities when an entity is linked to the fragment. Thus, the invention relates to compositions that comprise a carboxyterminal fragment of a HC of BoNT (hereinafter “HC”) linked to an entity. The invention also relates to methods of using such compositions.

In particular, the invention provides a composition for translocating an entity across a non-keratinized epithelium of an animal. The composition comprises an entity linked to a carboxyterminal fragment of the HC of a BoNT. The size of the entity is preferably not greater than the lumenal capacity of vesicles of cells of the epithelium. The selected entity for use in the composition of the invention may be immunogenic, therapeutic, and/or diagnostic in nature.

For example, within the scope of the invention is contemplated a composition that elicits an immune response (mucosal or systemic) against an antigen in a vertebrate. Such composition may contain at least one epitope of the antigen linked to a carboxyterminal fragment of the HC of a BoNT. The size of the epitope is preferably not greater than the lumenal capacity of vesicles of cells of the epithelium.

In an embodiment, the invention is a vaccine that includes an antigen linked to a carboxyterminal fragment of HC of a BoNT, wherein the antigen induces protective immunity against a pathogen of a vertebrate when the antigen is delivered to the circulation of the vertebrate. For example, the vaccine may comprise an antigen linked to a carboxyterminal fragment of a HC of a BoNT, wherein the antigen induces protective immunity against Clostridium botulinum neurotoxin in a vertebrate when the antigen is delivered to the circulation of the vertebrate.

In another aspect of the invention, a method of translocating an entity across a non-keratinized epithelium of an animal is provided. The method includes contacting an epithelium with a composition comprising the entity linked to a carboxyterminal fragment of the HC of a BoNT, wherein the size of the entity is not greater than the lumenal capacity of vesicles of cells of the epithelium.

Also contemplated are methods of inducing an immune response against an entity in a vertebrate. In such case, the methods involves: a) linking the entity to a carboxyterminal fragment of the HC of a BoNT, wherein the size of the entity is not greater than the lumenal capacity of vesicle cells of the epithelium; and b) contacting the fragment-linked entity with the epithelium.

Pharmaceutical compositions containing an entity linked to a carboxyterminal fragment of the HC of BoNT are also described herein, as are foodstuffs and translocating polypeptides that may be used in the methods and/or compositions of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there is shown in the drawings embodiments which may be presently preferred. It should be understood, however, that the invention is not limited to the precise arrangements, sequences, compounds, and instrumentalities shown.

FIG. 1 (consisting of FIGS. 1A to 1BB) is an alignment of the amino acid sequences of the Clostridium botulinum neurotoxin:

(i) serotype A HC (SEQ ID NO: 1, from GENBANK™ accession no. Q45894);

(ii) serotype B HC (SEQ ID NO: 2, from GENBANK™ accession no. P10844);

(iii) serotype C HC (SEQ ID NO: 3, from GENBANK™ accession no. P18640);

(iv) serotype D HC (SEQ ID NO: 4, from GENBANK™ accession no. P19321);

(v) serotype E HC (SEQ ID NO: 5, from GENBANK™ accession no. P30995);

(vi) serotype F HC (SEQ ID NO: 6, from GENBANK™ accession no. P30996); and

(vii) serotype G HC (SEQ ID NO: 7, from GENBANK™ accession no. Q60393). In the alignment, the amino acid sequence of the 88 kilodalton fragment of HC (serotype A) (SEQ ID NO: 8) (i.e., the fragment herein designated “88 kHC”, beginning at residue 524) is shown in bold text. The amino acid sequence of the 66 kilodalton fragment of HC (serotype A) (SEQ ID NO: 9) (i.e., the fragment herein designated “66 kHC”, beginning at residue 714) is shown in underlined text (part of which is doubly-underlined). The amino acid sequence of the 50 kilodalton fragment of HC (serotype A) (SEQ ID NO: 10) (i.e., the fragment herein designated “50 kHC”, beginning at residure 886) is shown in doubly-underlined text and the amino acid sequence of a forty-eight kilodalton portion of HC (serotype A) (SEQ ID NO: 169) (i.e., the 50 kHC fragment minus 2 kilodalton of its carboxy terminus, hereinafter designated “48 kHC”, beginning at residue 886 and ending at residue 1343) is shown in italicized text.

FIG. 2 is a schematic diagram illustrating the structure of native Clostridium botulinum (serotype A), its HC, and the relative positions of each of the fragments designated 88 kHC, 66 kHC, 50 kHC, and of the portion designated 48 kHC.

FIG. 3 is a Western blot of native botulinum neurotoxin type A (BoNT A) transcytosed in polarized gut epithelial cell cultures (T-84). The lanes represent: (A) Pre-transcytosis control for native toxin. (B) Native toxin transcytosed through T-84 cells (collected from basal chamber).

FIG. 4 is a Western blot of native botulinum neurotoxin type A transcytosed in polarized, canine kidney epithelial cell cultures (MDCK). The lanes represent: (A) Pre-transcytosis control for native toxin. (B) Native toxin transcytosed through MDCK cells (collected from basal chamber).

FIG. 5 is a Western blot of HC (HC) transcytosed in T-84 polarized epithelial cell cultures. The lanes represent: (A) Pre-transcytosis control for HC. (B) HC transcytosed through T-84 cells (collected from basal chamber).

FIG. 6 is a Western blot of HC transcytosed in polarized, canine kidney epithelial cell cultures (MDCK). The lanes represent: (A) Pre-transcytosis control for HC. (B) HC transcytosed through MDCK cells (collected from basal chamber).

FIG. 7 is a Western blot of 66 kDa HC carboxyterminal fragment (66 kHC) transcytosed in T-84 polarized epithelial cell cultures. The lanes represent: (A) Pre-transcytosis control for the 66 kHC. (B) The 66 kHC transcytosed through T-84 cells (collected from basal chamber).

FIG. 8 is a Western blot of 66 kDa HC carboxyterminal fragment (66 kHC) transcytosed in MDCK polarized canine kidney epithelial cell cultures. The lanes represent: (A) Pre-transcytosis control for the 66 kHC. (B) The 66 kHC transcytosed through MDCK cells (collected from basal chamber).

FIG. 9 is a Western blot of 50 kDa HC fragment (50 kHC) transcytosed in polarized epithelial cell cultures. The lanes represent: (A) Pre-transcytosis control for 50 kHC. (B) 50 kHC transcytosed through T-84 cells (collected from basal chamber).

FIG. 10 is a Western blot of 50 kHC fragment transcytosed in polarized, canine kidney epithelial cell cultures. The lanes represent: (A) Pre-transcytosis control for 50 kHC. (B) 50 kHC transcytosed through MDCK cells (collected from basal chamber).

FIG. 11 is a fluorescence emission spectrum of Alexa 568˜Botulinum toxin type A transcytosis in polarized T-84 epithelial cell cultures. In the Figure: ⋄=Alexa 568˜toxin;□=Culture medium.

FIG. 12 is a fluorescence emission spectrum of Alexa 568˜Botulinum toxin type A transcytosis in polarized MDCK epithelial cell cultures. In the Figure: ⋄=Alexa 568˜toxin; □=Culture medium.

FIG. 13 is a Western blot of biotin˜50 kHC transcytosed in polarized T-84 epithelial cell cultures. The lanes represent: (A) Pre-transcytosis control for biotin˜50 kHC. (B) biotin˜50 kHC transcytosed through T-84 cells (collected from basal chamber).

FIG. 14 is a Western blot of biotin˜50 kHC transcytosed in polarized MDCK epithelial cell cultures. The lanes represent: (A) Pre-transcytosis control for biotin˜50 kHC. (B) Biotin˜50 kHC transcytosed through MDCK cells (collected from basal chamber).

FIG. 15 is a fluorescence emission spectrum of GFP˜66 kHC transcytosis in polarized T-84 epithelial cell cultures. In the Figure: ●=GFP˜66 kHC; O=Culture medium.

FIG. 16 is a fluorescence emission spectrum of GFP˜66 kHC transcytosis in polarized MDCK epithelial cell cultures. In the Figure: ●=GFP˜66 kHC; O=Culture medium.

FIG. 17 is a Western blot of a S-Tag˜50 kHC conjugate transcytosed in T-84 polarized epithelial cell cultures. The lanes represent: (A) Pre-transcytosis control for the S-Tag˜50 kHC. (B) The S-Tag˜50 kHC transcytosed through T-84 cells (collected from basal chamber).

FIG. 18 is a Western blot of a S-Tag˜50 kHC conjugate transcytosed in MDCK polarized canine kidney epithelial cell cultures. The lanes represent: (A) Pre-transcytosis control for the S-Tag˜50 kHC. (B) The S-Tag˜50 kHC transcytosed through MDCK cells (collected from basal chamber).

FIG. 19 is a Western blot of GST˜88 kHC transcytosed in polarized T-84 epithelial cell cultures. The lanes represent: (A) Pre-transcytosis control for the GST˜88 kHC. (B) The GST˜88 kHC transcytosed through T-84 cells (collected from basal chamber).

FIG. 20 is a Western blot of GST˜88 kHC transcytosed in polarized MDCK epithelial cell cultures. The lanes represent: (A) Pre-transcytosis control for the GST˜88 kHC. (B) The GST˜88 kHC fragment fusion transcytosed through MDCK cells (collected from basal chamber).

FIG. 21 is a Western blot of GST˜66 kHC transcytosed in polarized T-84 epithelial cell cultures. The lanes represent: (A) Pre-transcytosis control for the GST˜66 kHC. (B) The GST˜66 kHC transcytosed through T-84 cells (collected from basal chamber).

FIG. 22 is a Western blot of GST˜66 kHC transcytosed in polarized MDCK epithelial cell cultures. The lanes represent: (A) Pre-transcytosis control for the GST˜66 kHC. (B) The GST˜66 kHC transcytosed through MDCK cells (collected from basal chamber).

FIG. 23 illustrates the blood level of botulinum neurotoxin serotype A after intranasal administration to mice.

FIG. 24 illustrates the blood level of serotype A HC after intranasal administration to mice.

FIG. 25 illustrates the blood level of 6×His-50 kHC after intranasal administration to mice.

FIG. 26 illustrates the blood level of GST˜50 kHC after intranasal administration to mice.

FIG. 27 is a Western blot of GST˜50 kHC fragment probed with immune serum obtained from mice immunized intranasally with GST˜50 kHC.

FIG. 28 illustrates enhanced immune responses for intranasal administration of 50 kHC by co-administration with cholera toxin B subunit (CTB). In the Figure: ▪=50 kHC; □=50 kHC +CTB.

FIG. 29 illustrates the development of a specific antibody response to 100 kDa HC after oral immunization in mice. ELISA titers obtained seven days after the second (boost 1), third (boost 2), and fourth (boost 3) administration of 100 kDa HC are shown.

FIG. 30, which consists of FIGS. 30A to 30C, is an alignment of the amino acid sequences of several seventeen kilodalton (17 kDa) hemagglutinin (“HA”) proteins associated with the botulinum toxin of various serotypes:

(1) 17 kDa HA, GENEBANK™ Accession No. CAA44260, SEQ ID NO: 20;

(2) 17 kDA HA, GENEBANK™ Accession No. BAA75081, SEQ ID NO: 21;

(3) 17 kDa HA, GENEBANK™ Accession No. P46083, SEQ ID NO: 22;

(4) 17 kDa HA, GENEBANK™ Accession No. BAA90658, SEQ ID NO: 23;

(5) 17 kDa HA, GENEBANK™ Accession No. BAB71742, SEQ ID NO: 24;

(6) 17 kDa HA, GENEBANK™ Accession No. BAA89710, SEQ ID NO: 25;

(7) 17 kDa HA, GENEBANK™ Accession No. S49104, SEQ ID NO: 26;

(8) 17 kDa HA, GENEBANK™ Accession No. AAA99056, SEQ ID NO: 27;

(9) 17 kDa HA, GENEBANK™ Accession No. CAA70496, SEQ ID NO: 28;

(10) 17 kDa HA, GENEBANK™ Accession No. AAB42188, SEQ ID NO: 29;

(11) 17 kDa HA, GENEBANK™ Accession No. CAA61226, SEQ ID NO: 30;

(12) 17 kDa HA, GENEBANK™ Accession No. B44644, SEQ ID NO: 31;

(13) 17 kDa HA, GENEBANK™ Accession No. AAB21357, SEQ ID NO: 32;

(14) 17 kDa HA, GENEBANK™ Accession No. S67990, SEQ ID NO: 33; and

(15) 17 kDa HA, GENEBANK™ Accession No. AAB21356, SEQ ID NO: 34.

FIG. 31, which consists of FIGS. 31A to 31D, is an alignment of the amino acid sequences of several twenty-one kilodalton (21 kDa) hemagglutinin (“HA”) proteins associated with the botulinum toxin of various serotypes:

(1) 21 kDa HA, GENEBANK™ Accession No. CAA55717, SEQ ID NO: 35;

(2) 21 kDa HA, GENEBANK™ Accession No. S68219, SEQ ID NO: 36;

(3) 21 kDa HA, GENEBANK™ Accession No. AAB42190, SEQ ID NO: 37;

(4) 21 kDa HA, GENEBANK™ Accession No. S58864, SEQ ID NO: 38;

(5) 21 kDa HA, GENEBANK™ Accession No. AAB64349, SEQ ID NO: 39;

(6) 21 kDa HA, GENEBANK™ Accession No. S58856, SEQ ID NO: 40;

(7) 21 kDa HA, GENEBANK™ Accession No. NB_{—}783832, SEQ ID NO: 41;

(8) 21 kDa HA, GENEBANK™ Accession No. CAA07094, SEQ ID NO: 42;

(9) 21 kDa HA, GENEBANK™ Accession No. CAA61227, SEQ ID NO : 43;

(10) 21 kDa HA, GENEBANK™ Accession No. CAA73969, SEQ ID NO: 44;

(11) 21 kDa HA, GENEBANK™ Accession No. CAA65350, SEQ ID NO: 45;

(12) 21 kDa HA, GENEBANK™ Accession No. CAA65347, SEQ ID NO: 46;

(13) 21 kDa HA, GENEBANK™ Accession No. CAA65345, SEQ ID NO: 47;

(14) 21 kDa HA, GENEBANK™ Accession No. BAA90656, SEQ ID NO: 48;

(15) 21 kDa HA, GENEBANK™ Accession No. BAA89708, SEQ ID NO: 49;

(16) 21 kDa HA, GENEBANK™ Accession No. S46426, SEQ ID NO: 50;

(17) 21 kDa HA, GENEBANK™ Accession No. CAA65346, SEQ ID NO: 51;

(18) 21 kDa HA, GENEBANK™ Accession No. BAA75074, SEQ ID NO: 52;

(19) 21 kDa HA, GENEBANK™ Accession No. BAB71744, SEQ ID NO: 53;

(20) 21 kDa HA, GENEBANK™ Accession No. CAC 19890, SEQ ID NO: 54;

(21) 21 kDa HA, GENEBANK™ Accession No. NP_{—}781286, SEQ ID NO: 55;

(22) 21 kDa HA, GENEBANK™ Accession No. JC5340, SEQ ID NO: 56; and

(23) 21 kDa HA, GENEBANK™ Accession No. AAK17956, SEQ ID NO: 57.

FIG. 32, which consists of FIGS. 32A to 32I, is an alignment of the amino acid sequences of several thirty-five kilodalton (35 kDa) hemagglutinin (“HA”) proteins associated with the botulinum toxin of various serotypes:

(1) 35 kDa HA, GENEBANK™ Accession No. AAA99055, SEQ ID NO: 58;

(2) 35 kDa HA, GENEBANK™ Accession No. S58865, SEQ ID NO: 59;

(3) 35 kDa HA, GENEBANK™ Accession No. CAA73965, SEQ ID NO: 60;

(4) 35 kDa HA, GENEBANK™ Accession No. H44644, SEQ ID NO: 61;

(5) 35 kDa HA, GENEBANK™ Accession No. S58857, SEQ ID NO: 62;

(6) 35 kDa HA, GENEBANK™ Accession No. AAB42189, SEQ ID NO: 63;

(7) 35 kDa HA, GENEBANK™ Accession No. CAA74632, SEQ ID NO: 64;

(8) 35 kDa HA, GENEBANK™ Accession No. BAB71747, SEQ ID NO: 65;

(9) 35 kDa HA, GENEBANK™ Accession No. BAA75077, SEQ ID NO: 66;

(10) 35 kDa HA, GENEBANK™ Accession No. S46429, SEQ ID NO: 67;

(11) 35 kDa HA, GENEBANK™ Accession No. P46084, SEQ ID NO: 68;

(12) 35 kDa HA, GENEBANK™ Accession No. BAA90659, SEQ ID NO: 69;

(13) 35 kDa HA, GENEBANK™ Accession No. BAA89711, SEQ ID NO: 70;

and

(14) 35 kDa HA, GENEBANK™ Accession No. CAA61226, SEQ ID NO: 71.

FIG. 33, which consists of FIGS. 33A to 33J, is an alignment of the amino acid sequences of several seventy kilodalton (70 kDa) hemagglutinin (“HA”) proteins associated with the botulinum toxin of various serotypes:

(1) 70 kDa HA, GENEBANK™ Accession No. BAA89709, SEQ ID NO: 72;

(2) 70 kDa HA, GENEBANK™ Accession No. BAA90657, SEQ ID NO: 73;

(3) 70 kDa HA, GENEBANK™ Accession No. AAB42187, SEQ ID NO: 74;

(4) 70 kDa HA, GENEBANK™ Accession No. AAM75949, SEQ ID NO: 75;

(5) 70 kDa HA, GENEBANK™ Accession No. CAA70495, SEQ ID NO: 76;

(6) 70 kDa HA, GENEBANK™ Accession No. CAA73963, SEQ ID NO: 77;

(7) 70 kDa HA, GENEBANK™ Accession No. CAA61225, SEQ ID NO: 78;

(8) 70 kDa HA, GENEBANK™ Accession No. CAA75931, SEQ ID NO: 79;

(9) 70 kDa HA, GENEBANK™ Accession No. QLCLBF, SEQ ID NO: 80;

(10) 70 kDa HA, GENEBANK™ Accession No. AAA72120, SEQ ID NO: 81;

(11) 70 kDa HA, GENEBANK™ Accession No. CAA04327, SEQ ID NO: 82;

(12) 70 kDa HA, GENEBANK™ Accession No. CAA57443, SEQ ID NO: 83;

(13) 70 kDa HA, GENEBANK™ Accession No. AAA99057, SEQ ID NO: 84;

(14) 70 kDa HA, GENEBANK™ Accession No. P01558, SEQ ID NO: 85;

(15) 70 kDa HA, GENEBANK™ Accession No. BAA07575, SEQ ID NO: 170;

(16) 70 kDa HA, GENEBANK™ Accession No. P46085, SEQ ID NO: 171;

and

(17) 70 kDa HA, GENEBANK™ Accession No. BAA75075, SEQ ID NO: 188.

FIG. 34, which consists of FIGS. 34A to 34AA, is an alignment of the amino acid sequences of several nontoxin, non-hemagglutinin protein (“NTNH”) proteins associated with the botulinum toxin of various serotypes:

(1) NTNH, GENEBANK™ Accession No. CAA55073, SEQ ID NO: 86;

(2) NTNH, GENEBANK™ Accession No. CAA73967, SEQ ID NO: 87;

(3) NTNH, GENEBANK™ Accession No. CAA55074, SEQ ID NO: 88;

(4) NTNH, GENEBANK™ Accession No. AAB64350, SEQ ID NO: 89;

(5) NTNH, GENEBANK™ Accession No. CAA61125, SEQ ID NO: 90;

(6) NTNH, GENEBANK™ Accession No. CAA74634, SEQ ID NO: 91;

(7) NTNH, GENEBANK™ Accession No. S68218, SEQ ID NO: 92;

(8) NTNH, GENEBANK™ Accession No. CAA63550, SEQ ID NO: 93;

(9) NTNH, GENEBANK™ Accession No. AAB42191, SEQ ID NO: 94;

(10) NTNH, GENEBANK™ Accession No. CAA61228, SEQ ID NO: 95;

(11) NTNH, GENEBANK™ Accession No. BAA90660, SEQ ID NO: 96;

(12) NTNH, GENEBANK™ Accession No. BAA89712, SEQ ID NO: 97;

(13) NTNH, GENEBANK™ Accession No. BAB71748, SEQ ID NO: 98;

(14) NTNH, GENEBANK™ Accession No. BAA75083, SEQ ID NO: 99;

(15) NTNH, GENEBANK™ Accession No. S46430, SEQ ID NO: 100;

(16) NTNH, GENEBANK™ Accession No. AAB36016, SEQ ID NO: 101;

(17) NTNH, GENEBANK™ Accession No. P46081, SEQ ID NO: 102;

(18) NTNH, GENEBANK™ Accession No. JC4901, SEQ ID NO: 103;

(19) NTNH, GENEBANK™ Accession No. BAA12299, SEQ ID NO: 104;

(20) NTNH, GENEBANK™ Accession No. CAA74630, SEQ ID NO: 105;

(21) NTNH, GENEBANK™ Accession No. CAA61123, SEQ ID NO: 106;

(22) NTNH, GENEBANK™ Accession No. CAA67511, SEQ ID NO: 107;

(23) NTNH, GENEBANK™ Accession No. CAA61233, SEQ ID NO: 108;

(24) NTNH, GENEBANK™ Accession No. AAC60474, SEQ ID NO: 109;

(25) NTNH, GENEBANK™ Accession No.I40644, SEQ ID NO: 110;

(26) NTNH, GENEBANK™ Accession No. CAA7397 1, SEQ ID NO: 111;

(27) NTNH, GENEBANK™ Accession No. CAA72807, SEQ ID NO: 112;

(28) NTNH, GENEBANK™ Accession No. P46082, SEQ ID NO: 113;

(29) NTNH, GENEBANK™ Accession No. A47708, SEQ ID NO: 114;

(30) NTNH, GENEBANK™ Accession No. Q06366, SEQ ID NO: 115;

(31) NTNH, GENEBANK™ Accession No. AAM75953, SEQ ID NO: 116;

(32) NTNH, GENEBANK™ Accession No. I40631, SEQ ID NO: 117;

(33) NTNH, GENEBANK™ Accession No. P10844, SEQ ID NO: 118;

(34) NTNH, GENEBANK™ Accession No. 1EPWA, SEQ ID NO: 119;

(35) NTNH, GENEBANK™ Accession No. BAA75078, SEQ ID NO: 120;

(36) NTNH, GENEBANK™ Accession No. AAL11498, SEQ ID NO: 121;

(37) NTNH, GENEBANK™ Accession No. AAK97132, SEQ ID NO: 122;

(38) NTNH, GENEBANK™ Accession No. CAA73968, SEQ ID NO: 123;

(39) NTNH, GENEBANK™ Accession No. P30995, SEQ ID NO: 124;

(40) NTNH, GENEBANK™ Accession No. BAC05434, SEQ ID NO: 125;

(41) NTNH, GENEBANK™ Accession No. BAB12249, SEQ ID NO: 126;

(42) NTNH, GENEBANK™ Accession No. BAB03512, SEQ ID NO: 127;

(43) NTNH, GENEBANK™ Accession No. S21178, SEQ ID NO: 128;

(44) NTNH, GENEBANK™ Accession No. Q00496, SEQ ID NO: 129;

(45) NTNH, GENEBANK™ Accession No. Q45894, SEQ ID NO: 130;

(46) NTNH, GENEBANK™ Accession No. CAA65352, SEQ ID NO: 131;

(47) NTNH, GENEBANK™ Accession No. CAA65348, SEQ ID NO: 132;

(48) NTNH, GENEBANK™ Accession No. AAB22656, SEQ ID NO: 133;

(49) NTNH, GENEBANK™ Accession No. CAA71744, SEQ ID NO: 134;

(50) NTNH, GENEBANK™ Accession No. A49777, SEQ ID NO: 135;

(51) NTNH, GENEBANK™ Accession No. P18640, SEQ ID NO: 136;

(52) NTNH, GENEBANK™ Accession No. 1F83A, SEQ ID NO: 137;

(53) NTNH, GENEBANK™ Accession No. 1F82A, SEQ ID NO: 138;

(54) NTNH, GENEBANK™ Accession No. BAA89713, SEQ ID NO: 139;

(55) NTNH, GENEBANK™ Accession No. BAA08418, SEQ ID NO: 140;

(56) NTNH, GENEBANK™ Accession No. A49928, SEQ ID NO: 141;

(57) NTNH, GENEBANK™ Accession No. BAC22064, SEQ ID NO: 142;

(58) NTNH, GENEBANK™ Accession No. 1906297A, SEQ ID NO: 143;

(59) NTNH, GENEBANK™ Accession No. AAC37720, SEQ ID NO: 144;

(60) NTNH, GENEBANK™ Accession No. NP_{—}783831, SEQ ID NO: 145;

(61) NTNH, GENEBANK™ Accession No. S39791, SEQ ID NO: 146;

(62) NTNH, GENEBANK™ Accession No. 1906297B, SEQ ID NO: 147;

(63) NTNH, GENEBANK™ Accession No. CAA37321, SEQ ID NO: 148;

(64) NTNH, GENEBANK™ Accession No. AAK72964, SEQ ID NO: 149;

(65) NTNH, GENEBANK™ Accession No. Q60393, SEQ ID NO: 150;

(66) NTNH, GENEBANK™ Accession No. AA021363, SEQ ID NO: 151;

(67) NTNH, GENEBANK™ Accession No. AAA23210, SEQ ID NO: 152;

(68) NTNH, GENEBANK™ Accession No. CAA61124, SEQ ID NO: 153;

(69) NTNH, GENEBANK™ Accession No. AAL66183, SEQ ID NO: 154;

(70) NTNH, GENEBANK™ Accession No. 1717342A, SEQ ID NO: 155;

(71) NTNH, GENEBANK™ Accession No. S33411, SEQ ID NO: 156;

(72) NTNH, GENEBANK™ Accession No. 3BTAA, SEQ ID NO: 157;

(73) NTNH, GENEBANK™ Accession No. CAA36289, SEQ ID NO: 158;

(74) NTNH, GENEBANK™ Accession No. P10845, SEQ ID NO: 159;

(75) NTNH, GENEBANK™ Accession No. BTCLAB, SEQ ID NO: 160;

(76) NTNH, GENEBANK™ Accession No. AAD09563, SEQ ID NO: 161;

(77) NTNH, GENEBANK™ Accession No. CAA73972, SEQ ID NO: 162;

(78) NTNH, GENEBANK™ Accession No. CAA77991, SEQ ID NO: 163;

(79) NTNH, GENEBANK™ Accession No. AAB24244, SEQ ID NO: 164;

(80) NTNH, GENEBANK™ Accession No. BAA90661, SEQ ID NO: 165;

(81) NTNH, GENEBANK™ Accession No. S70582, SEQ ID NO: 166; and

(82) NTNH, GENEBANK™ Accession No. BAA75084, SEQ ID NO: 167;

and

(83) NTNH, GENEBANK™ Accession No. P19321, SEQ ID NO: 168.

(84) NTNH, GENEBANK™ Accession No. S48110, SEQ ID NO: 173;

(85) NTNH, GENEBANK™ Accession No. S48109, SEQ ID NO: 174;

(86) NTNH, GENEBANK™ Accession No. CAA50145, SEQ ID NO: 175;

(87) NTNH, GENEBANK™ Accession No. CAA50150, SEQ ID NO: 176;

(88) NTNH, GENEBANK™ Accession No. AAA23282, SEQ ID NO: 177;

(89) NTNH, GENEBANK™ Accession No. AAG01403, SEQ ID NO: 178;

(90) NTNH, GENEBANK™ Accession No. AAA80610, SEQ ID NO: 179;

(91) NTNH, GENEBANK™ Accession No. 1DIWA, SEQ ID NO: 180;

(92) NTNH, GENEBANK™ Accession No. 1FV2A, SEQ ID NO: 181;

(93) NTNH, GENEBANK™ Accession No. 1D0HA, SEQ ID NO: 182;

(94) NTNH, GENEBANK™ Accession No. 1DLLA, SEQ ID NO: 183;

(95) NTNH, GENEBANK™ Accession No. 1AF9, SEQ ID NO: 184;

(96) NTNH, GENEBANK™ Accession No. 1DFQA, SEQ ID NO: 185;

(97) NTNH, GENEBANK™ Accession No. AAF73267, SEQ ID NO: 186;

(98) NTNH, GENEBANK™ Accession No. AAA230209, SEQ ID NO: 187;

and

(99) NTNH, GENEBANK™ Accession No. CAB43706. SEQ ID NO: 188.

### DETAILED DESCRIPTION OF THE INVENTION

Translocation of BoNTs (the holotoxin) across epithelial membranes is believed to occur by binding of the BoNT to the membrane of an epithelial cell, invagination of the cell's membrane resulting in enclosure of the BoNT within a vesicle of the cell, translocation of the vesicle from one side of the cell to the other (e.g., from the apical face of the cell to its basolateral face, or vice versa), re-integration of the vesicle with the cell's membrane and release of the BoNT from the cell. It has been discovered that some carboxyterminal fragments of HCs share with naturally-occurring BoNTs the ability to translocate (i.e., transcytose) across epithelial membranes without entering the cytosol of epithelial cells. In particular, the inventors have discovered that HC carboxyterminal fragments as small as about 2 kilodaltons retain epithelial transcytotic capacity and can be used to ferry entities as large as 1000 daltons or greater across epithelial membranes.

The nature of the entity linked to the carboxyterminal HC fragment does not significantly affect the fragment's transcytotic capacity. Substantially any type of entity can be transported in this manner, limited only by the size capacity of the epithelial vesicles and by one's ability to link the entity to the HC fragment.

The invention includes a composition for translocating an entity across an animal's non-keratinized epithelium. The composition comprises an entity linked to a carboxyterminal HC fragment. It is preferred that the size of the entity is not greater than the lumenal capacity of vesicles of cells of the epithelium. The epithelium should be a non-keratinized epithelium, and is preferably not kidney epithelium. The fragment-linked entity(ies) can be suspended or mixed with a variety of other ingredients, such as pharmaceutically acceptable vehicles, the protective auxiliary proteins HA and/or NTNH which ordinarily accompany the active botulinum neurotoxin, fillers and/or other components commonly used in pharmaceutical preparations.

As used herein, the terms “carboxyterminal fragment of the HC of Clostridium botulinum neurotoxin” or “carboxyterminal HC fragment” means a fragment of the amino acid sequence of a full length HC of BoNT of any serotype (e.g., the heavy chain of SEQ ID NOs: 1-7), the fragment including at least a portion of the sequence that makes up that half of the full length HC amino acid sequence that includes that carboxy terminus. Accordingly, it is contemplated that the carboxyterminal fragment for use in the invention is shorter than the full length HC. The fragment may exclude, for example, at least one amino acid residue of the full length HC, and preferably excludes 50, 100, 150, 200, 250, 300, 350, or 400 or more residues of the full length HC. Preferably, many of the excluded residues are those that occur in that half of the full length sequence that includes the amino terminus of the full length HC. Nonetheless, embodiments of the fragment are contemplated in which 5, 10, 15, 25, 50, or more amino acid residues are also omitted from that half of the full length HC that include the carboxy terminus.

In an embodiment of the invention, the carboxyterminal HC fragment of the invention includes about sixty residues of the HC of the Clostridium botulinum neurotoxin, with a preferred fragment including about thirty-five residues HC of the Clostridium botulinum neurotoxin and a more preferred fragment including twenty to about fifty amino acid residues of the HC of the Clostridium botulinum neurotoxin.

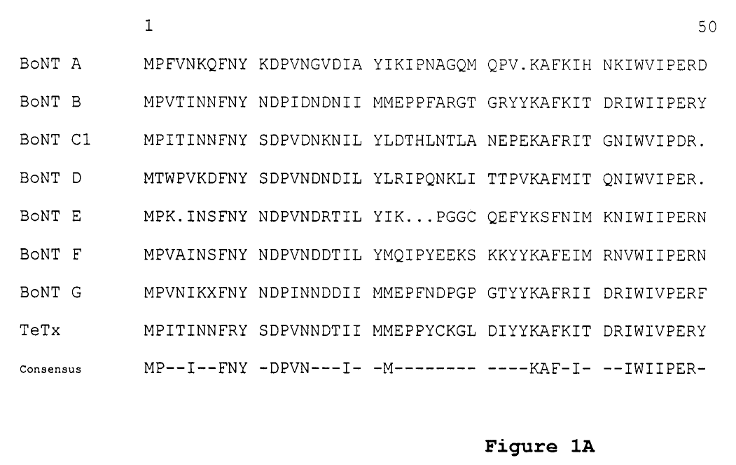

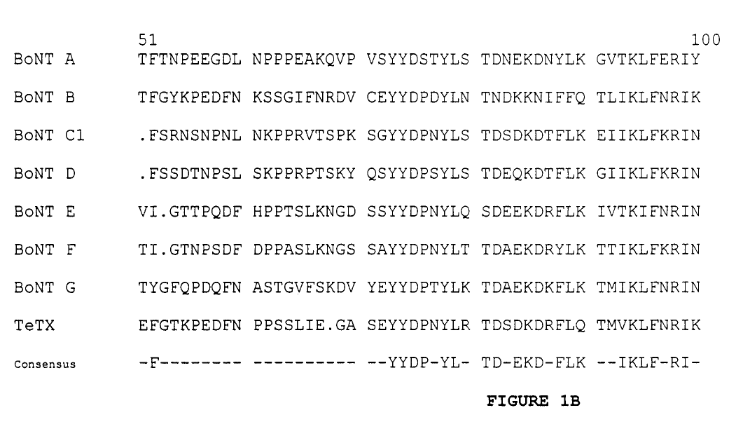

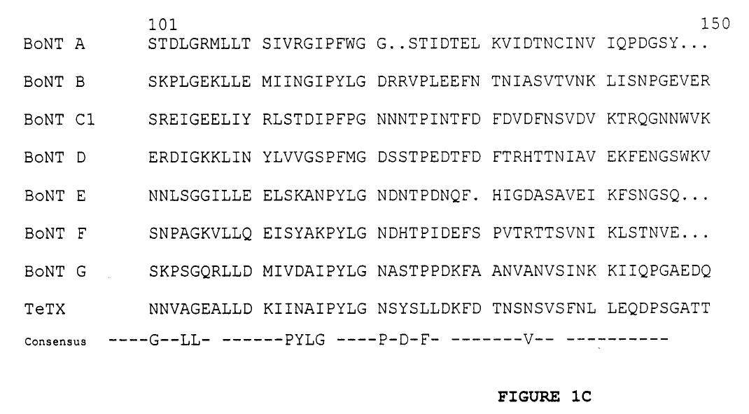

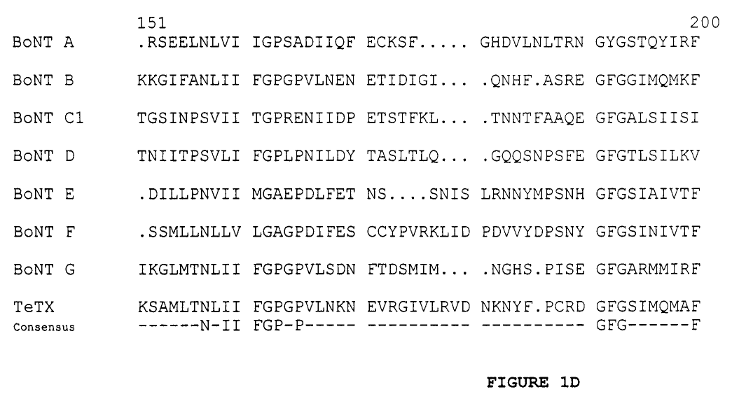

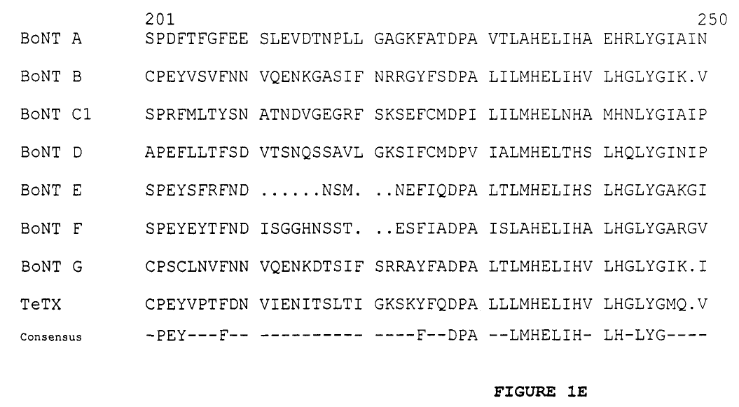

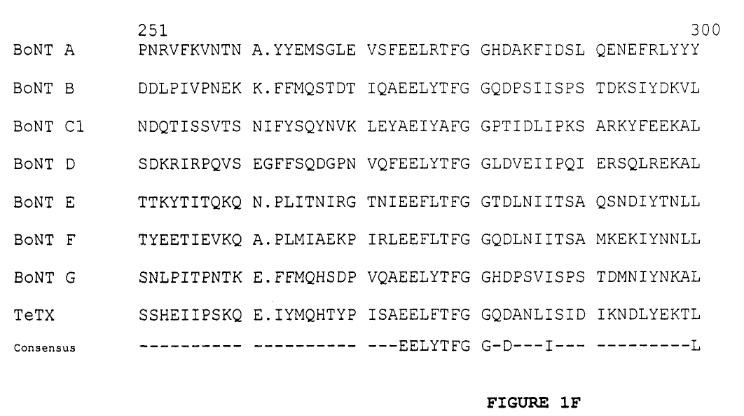

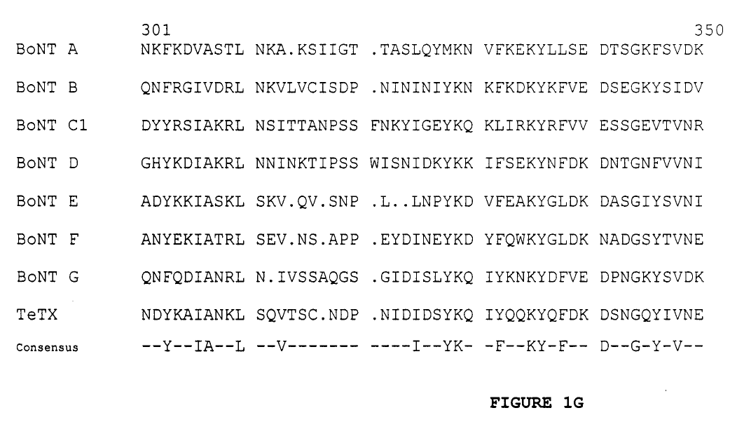

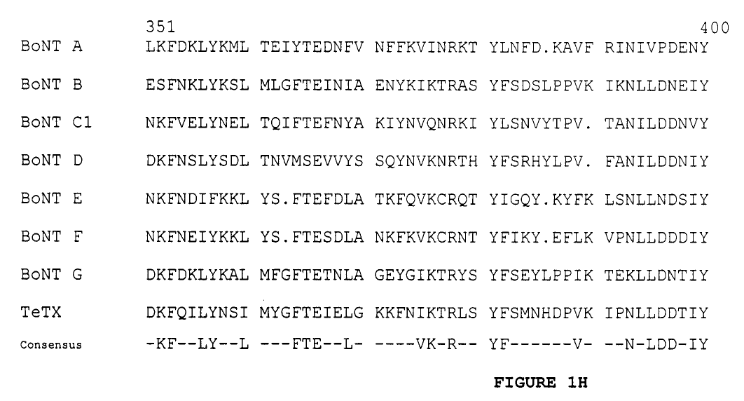

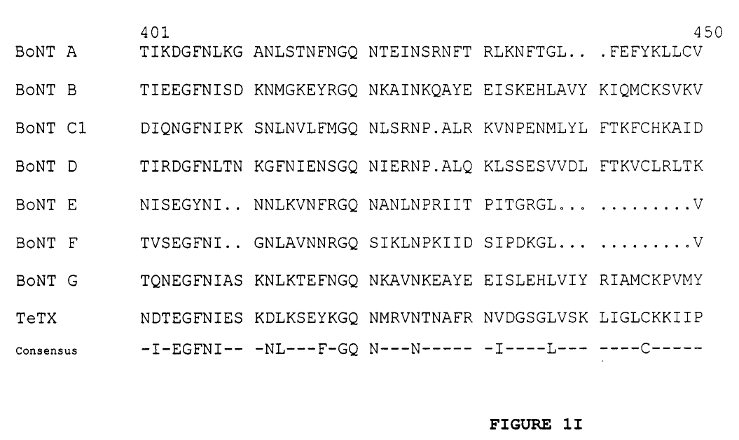

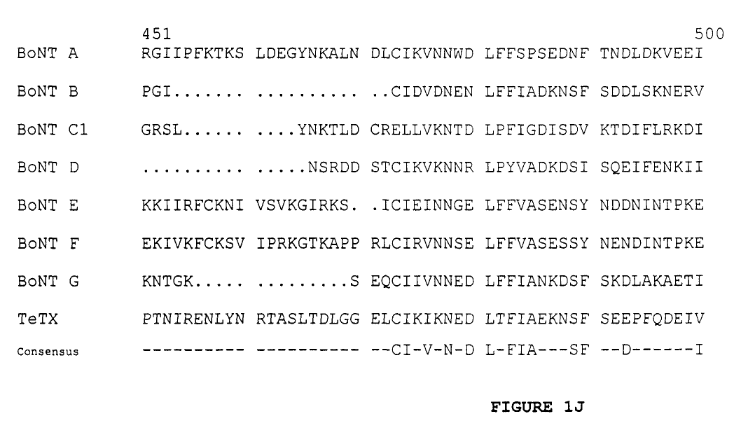

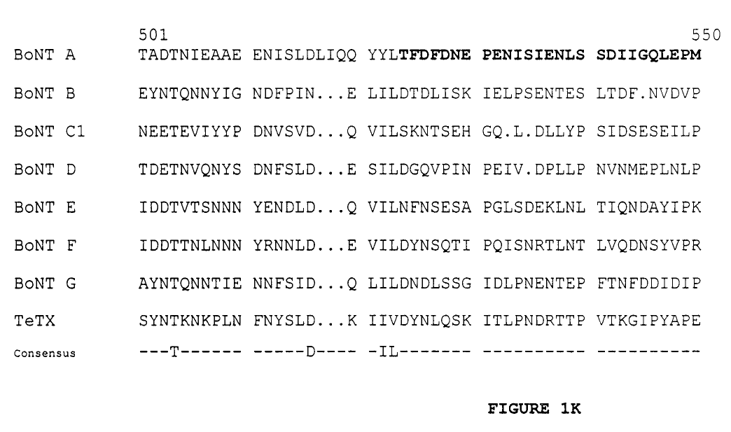

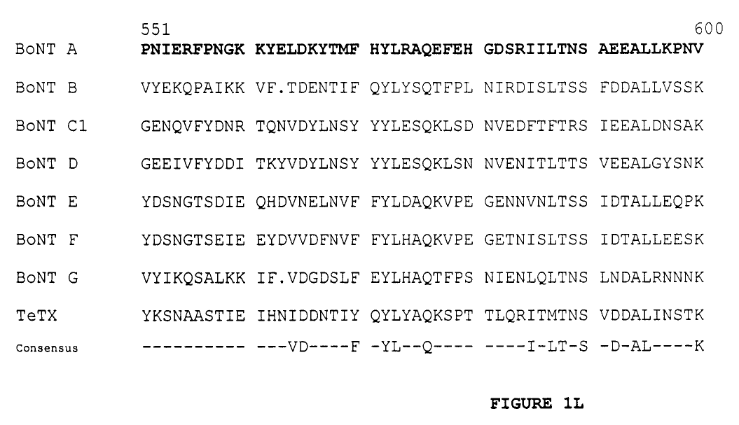

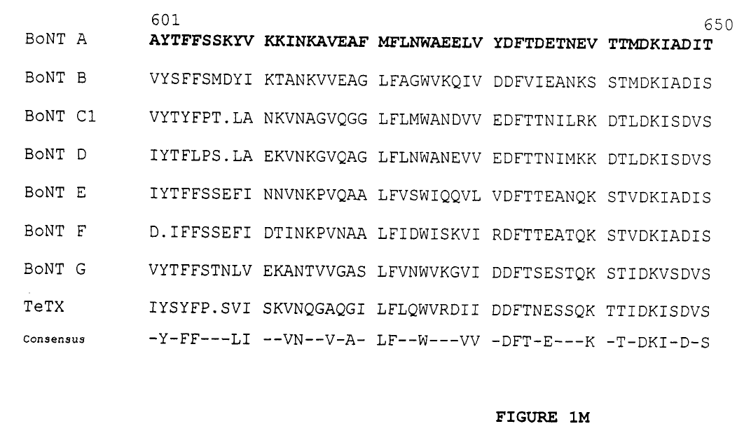

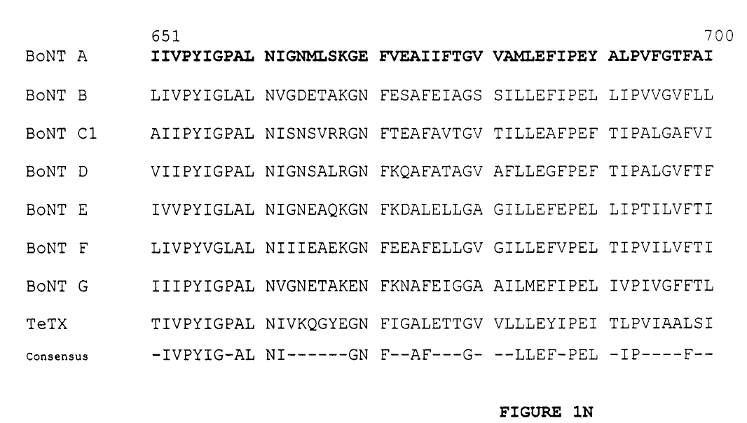

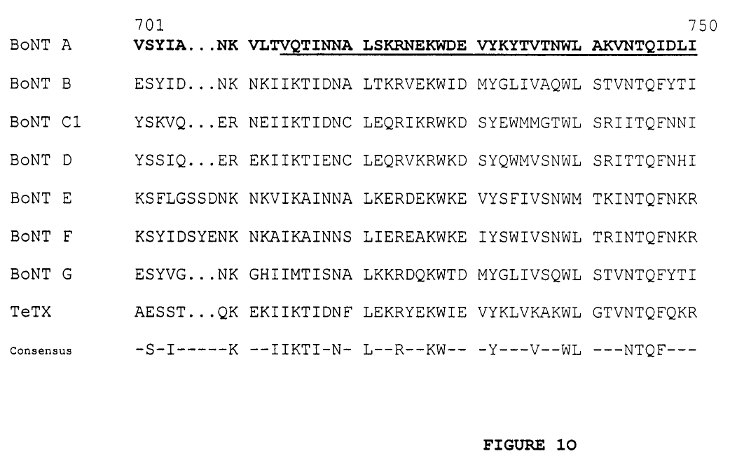

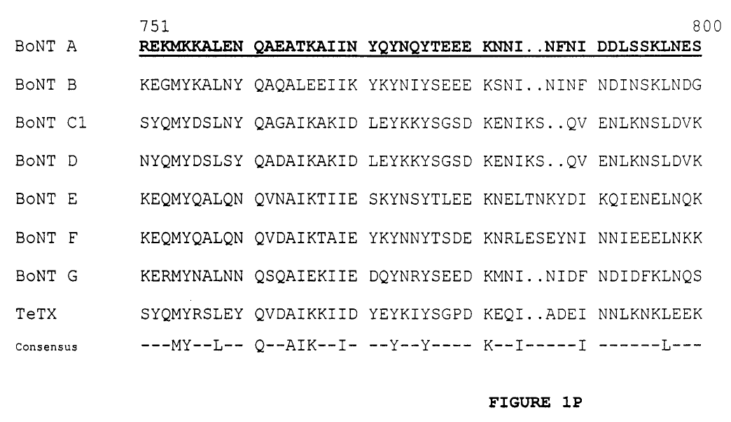

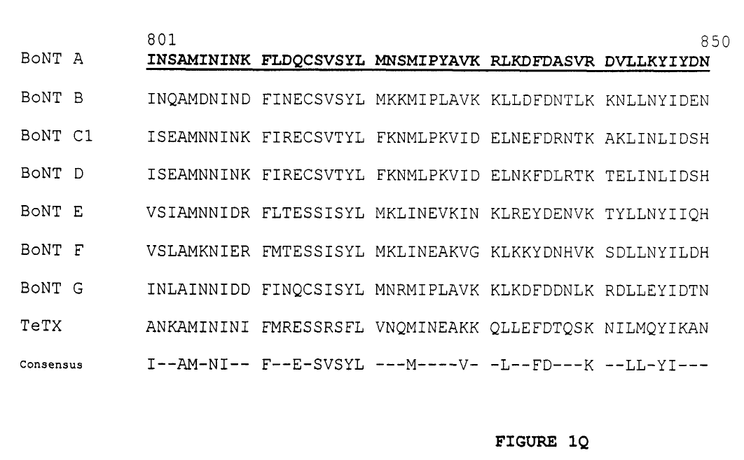

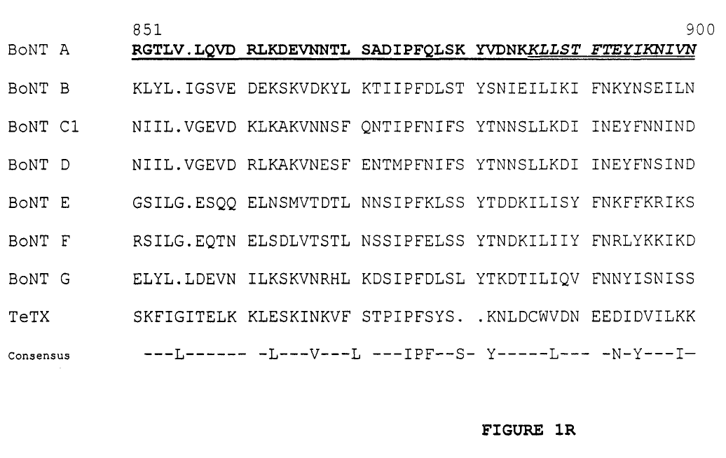

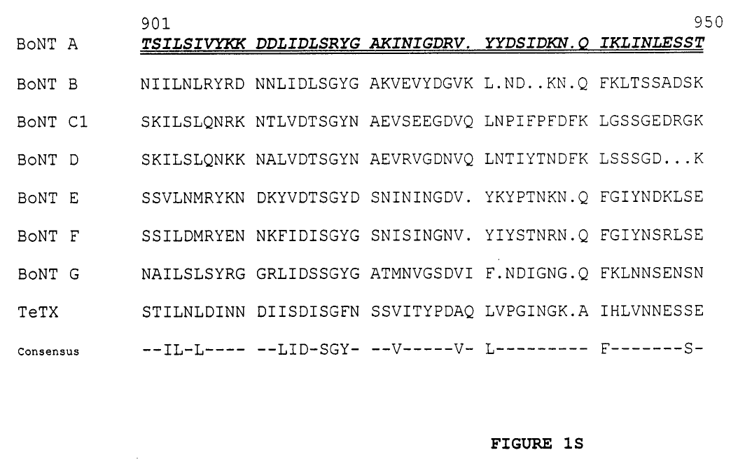

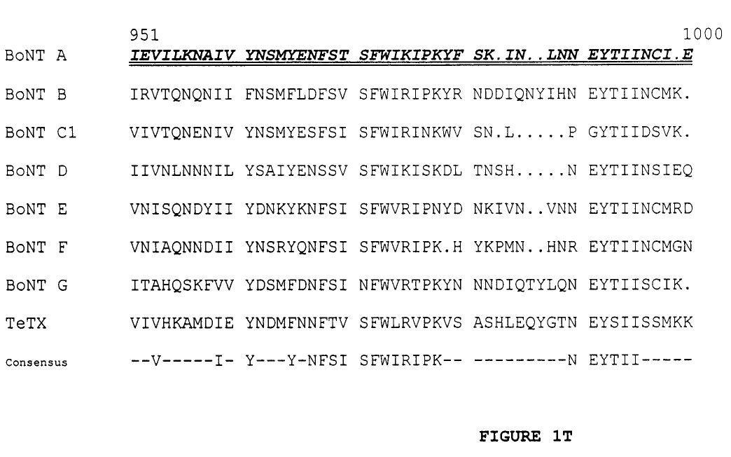

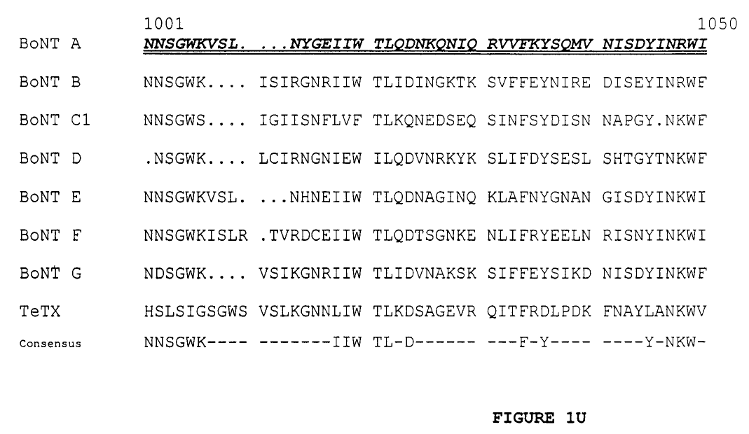

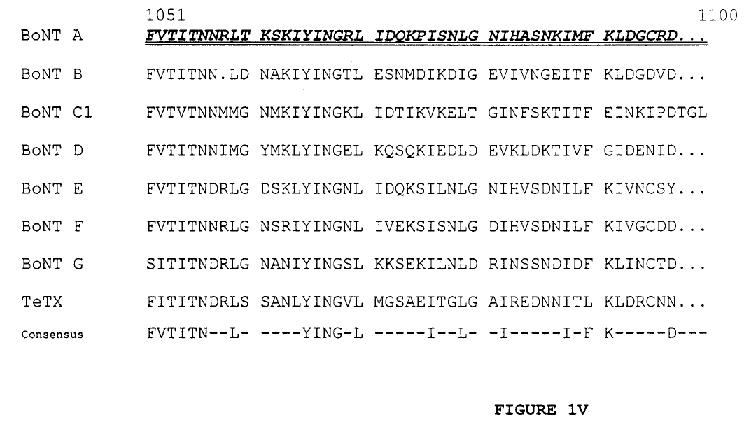

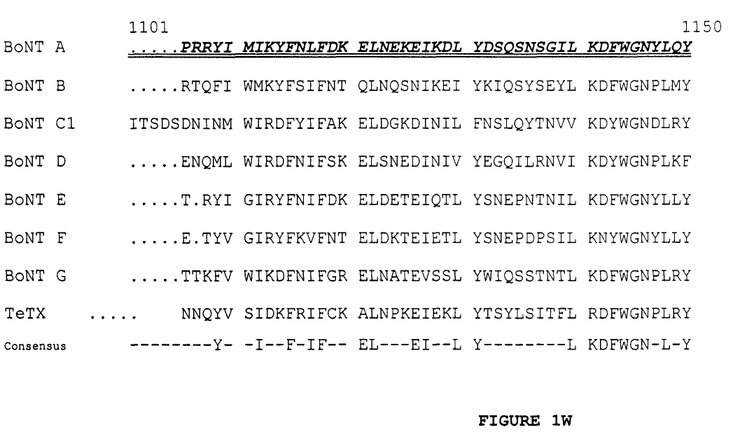

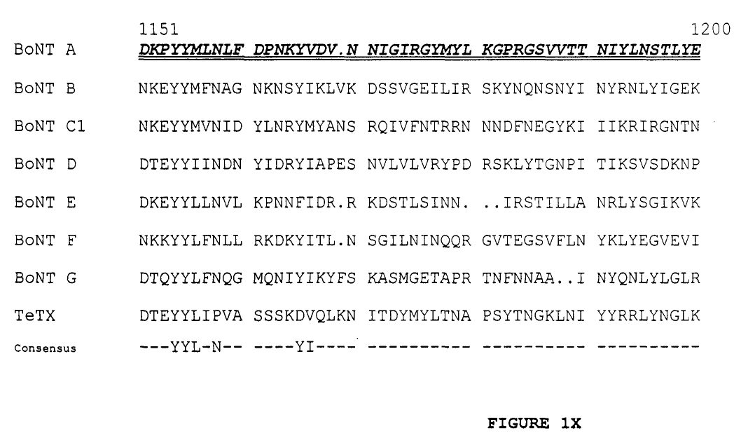

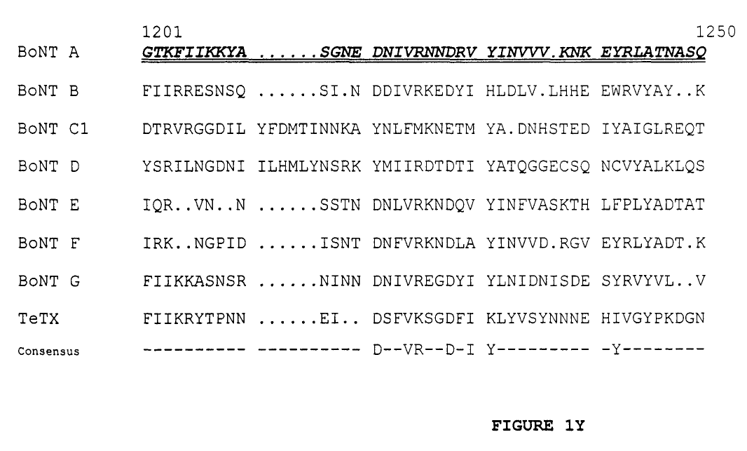

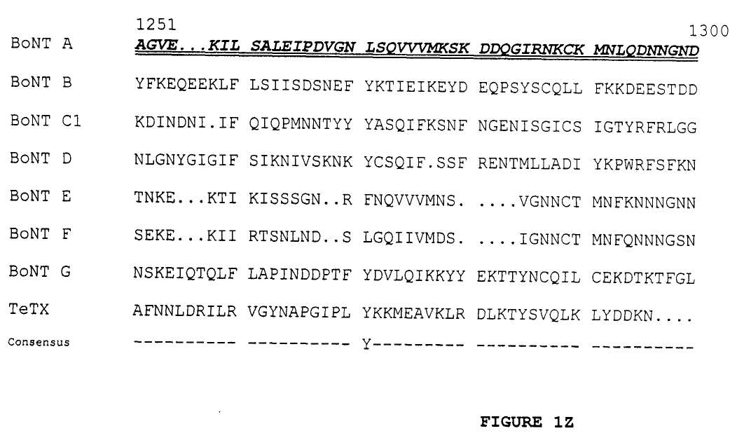

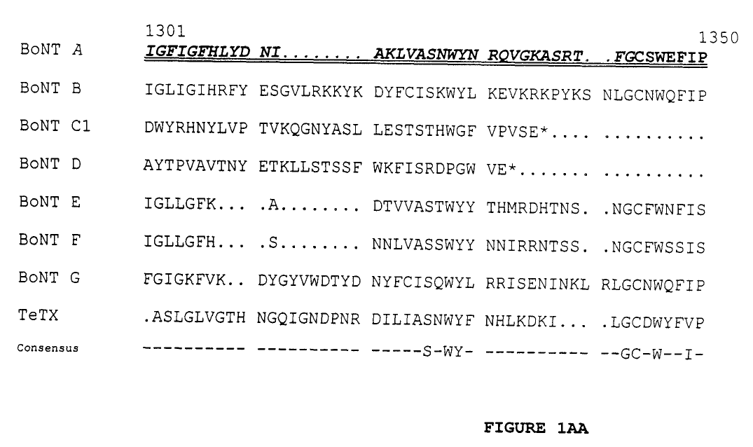

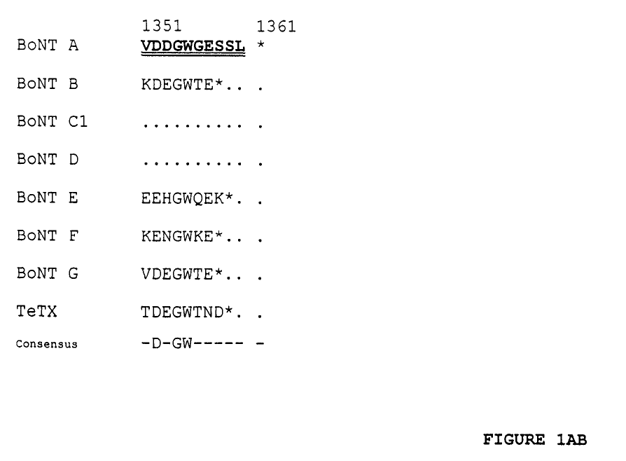

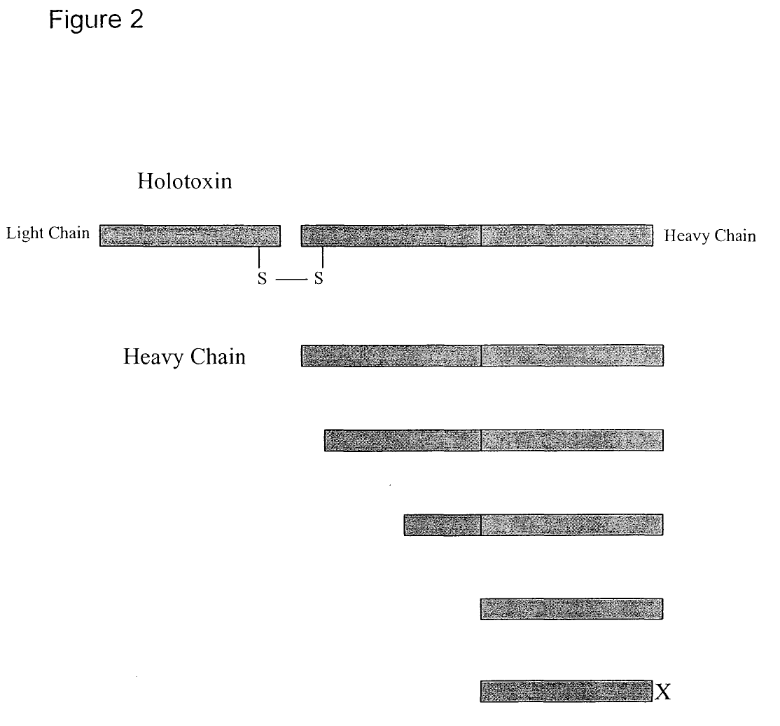

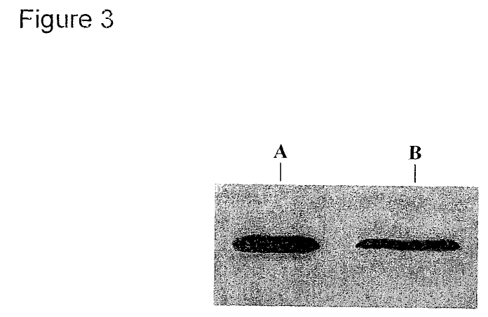

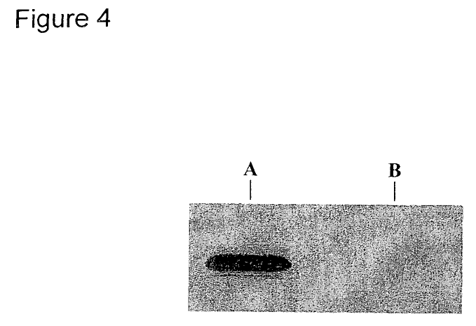

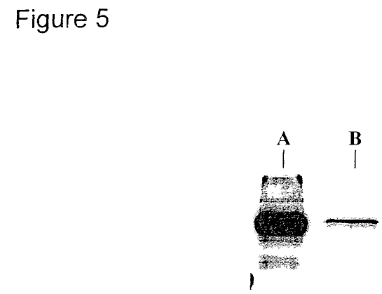

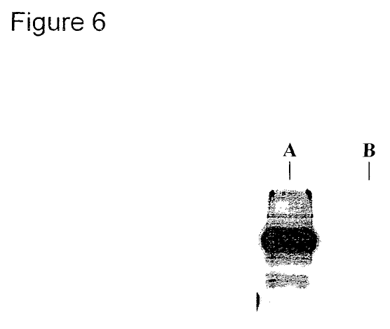

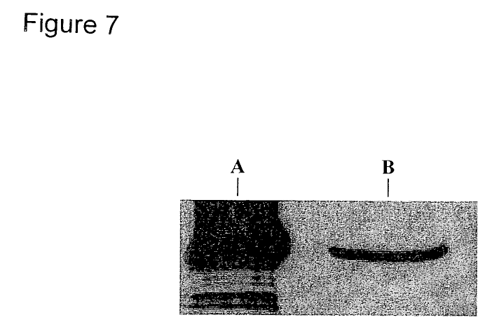

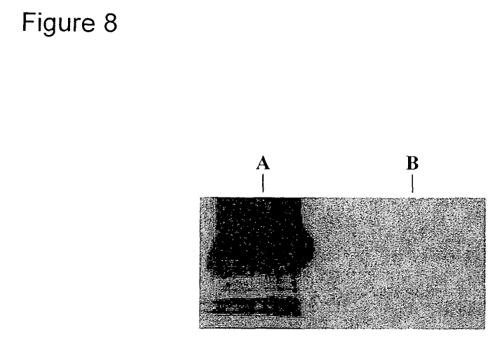

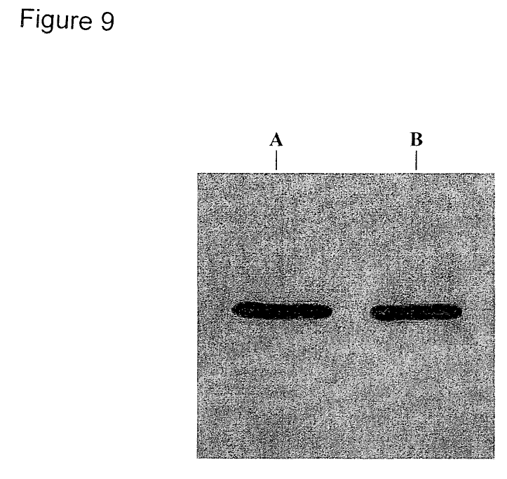

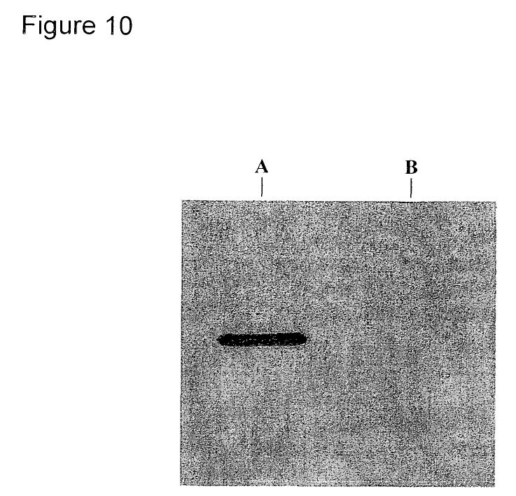

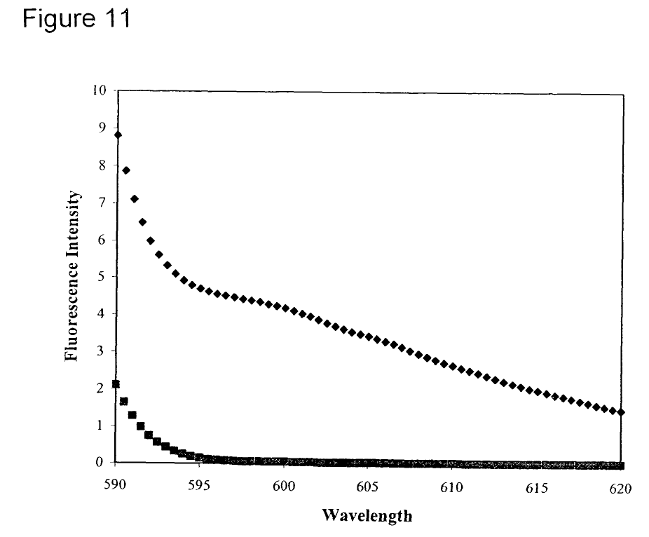

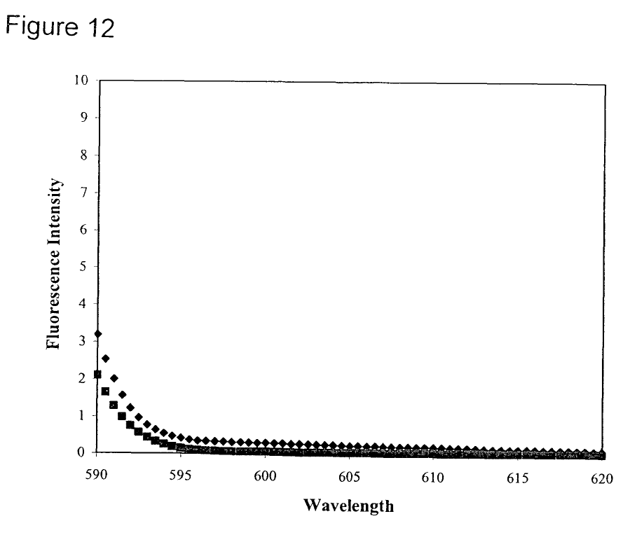

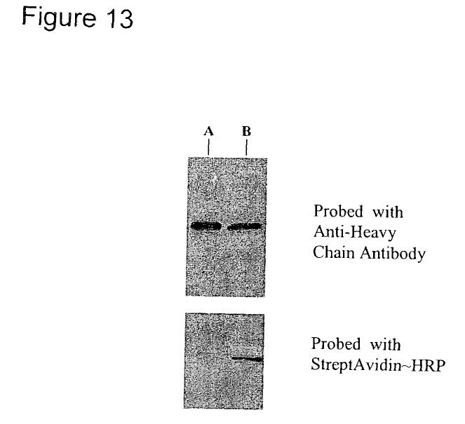

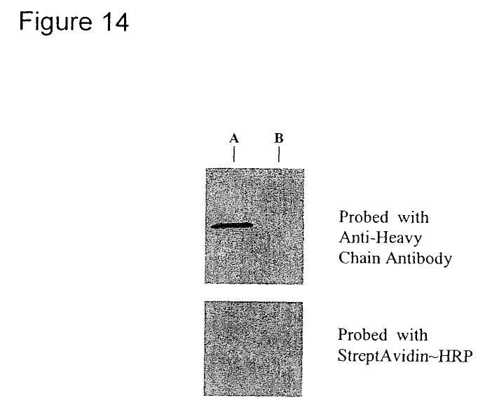

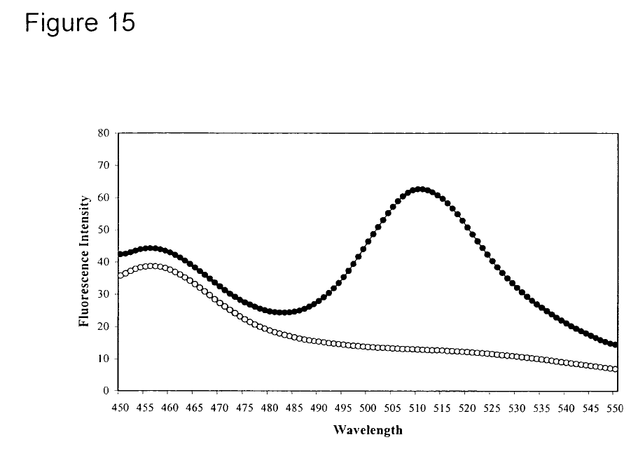

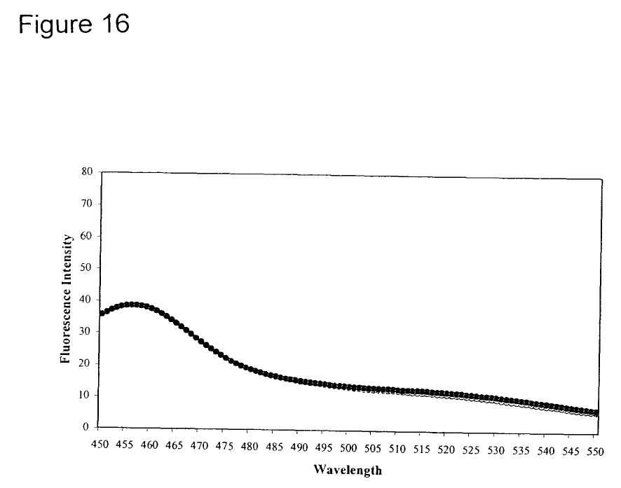

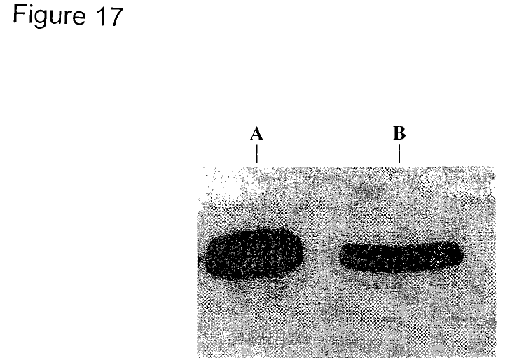

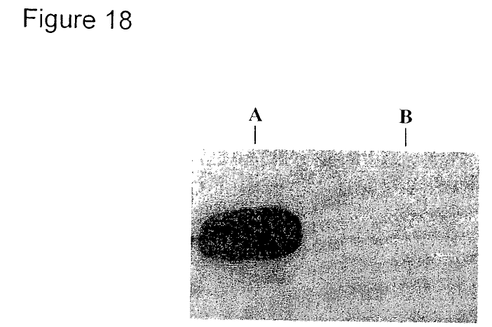

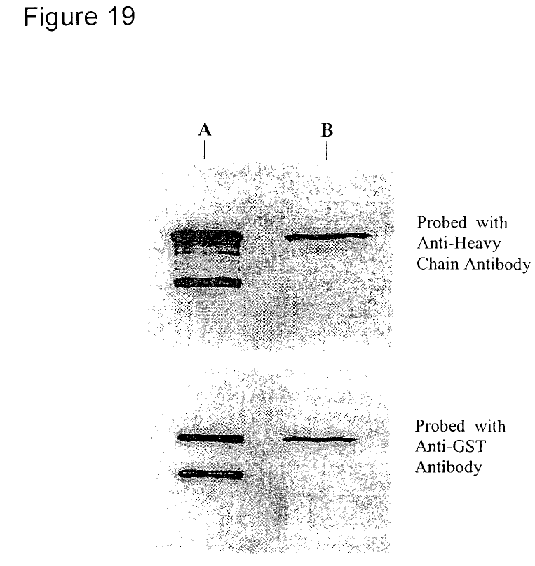

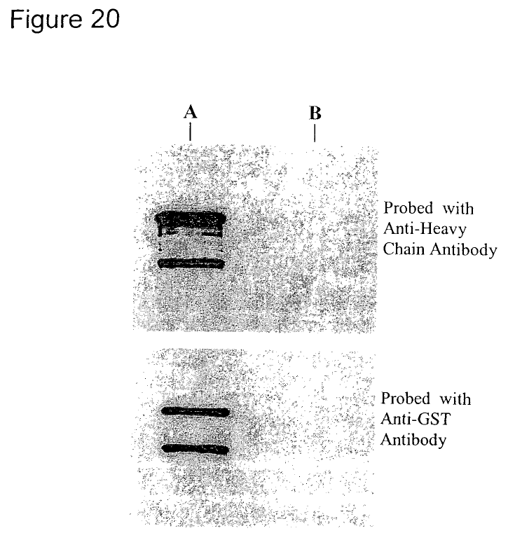

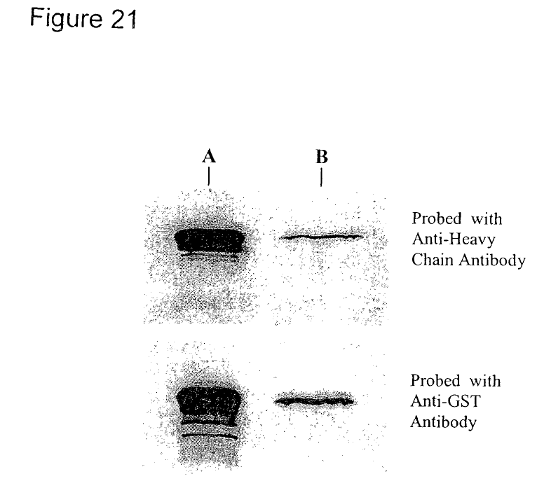

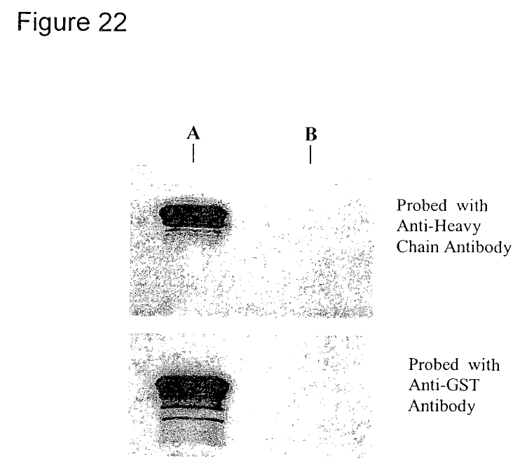

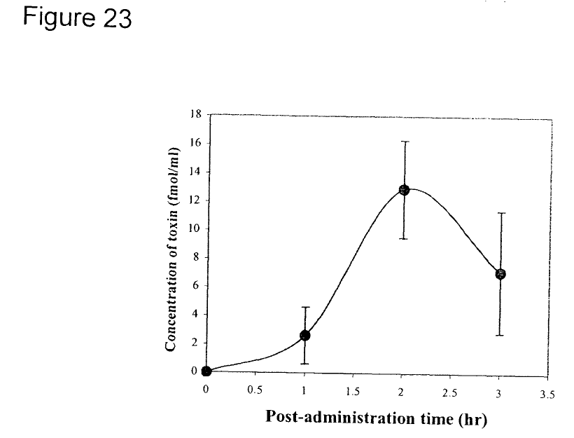

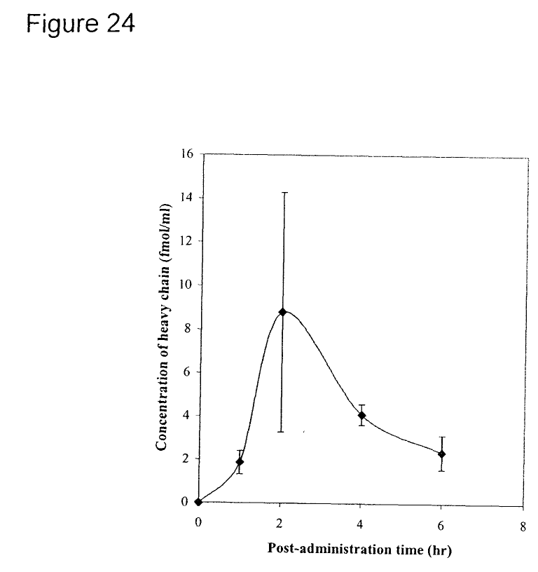

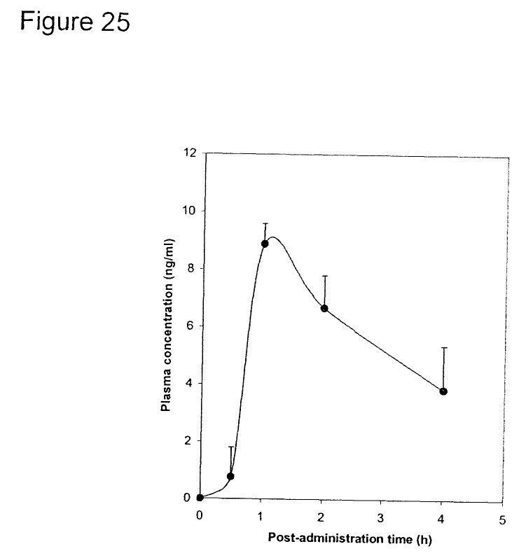

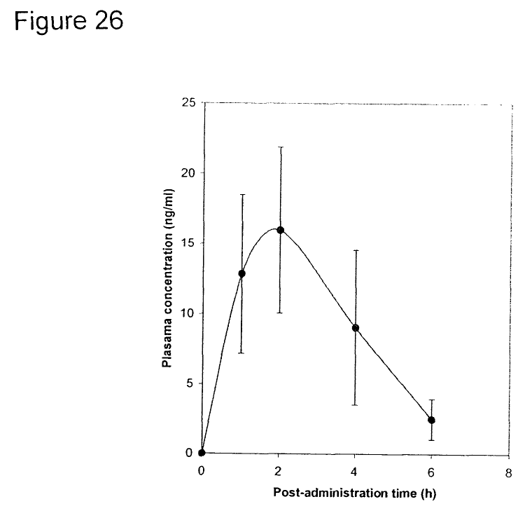

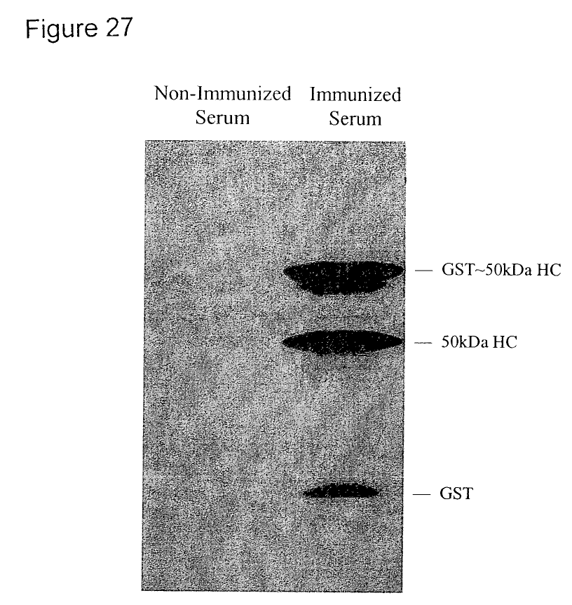

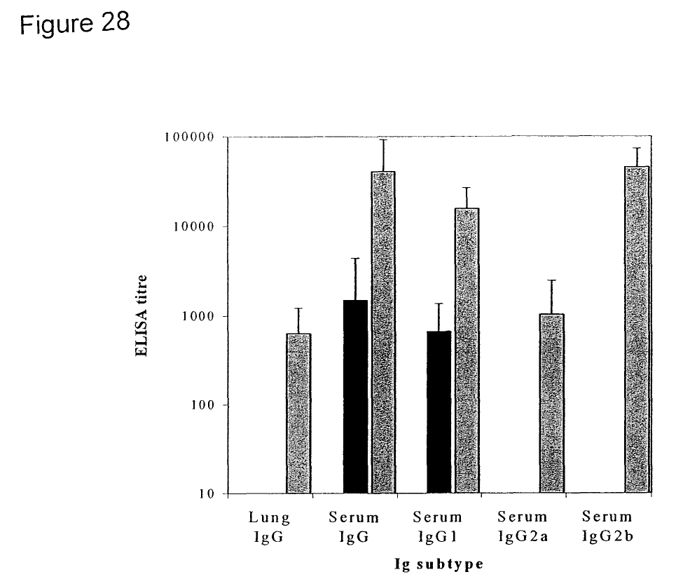

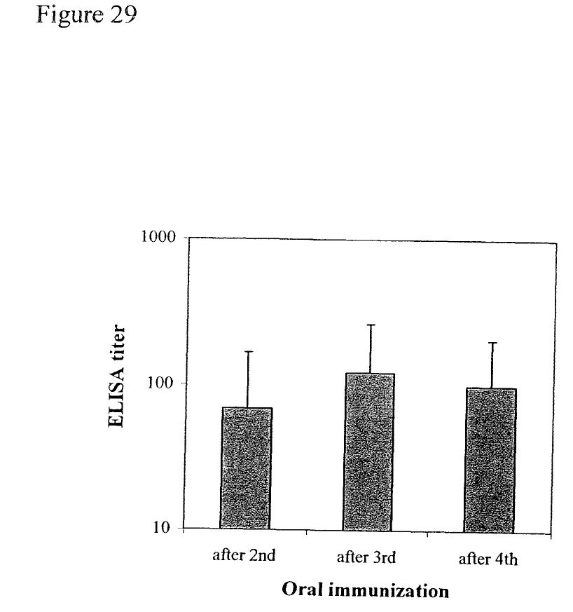

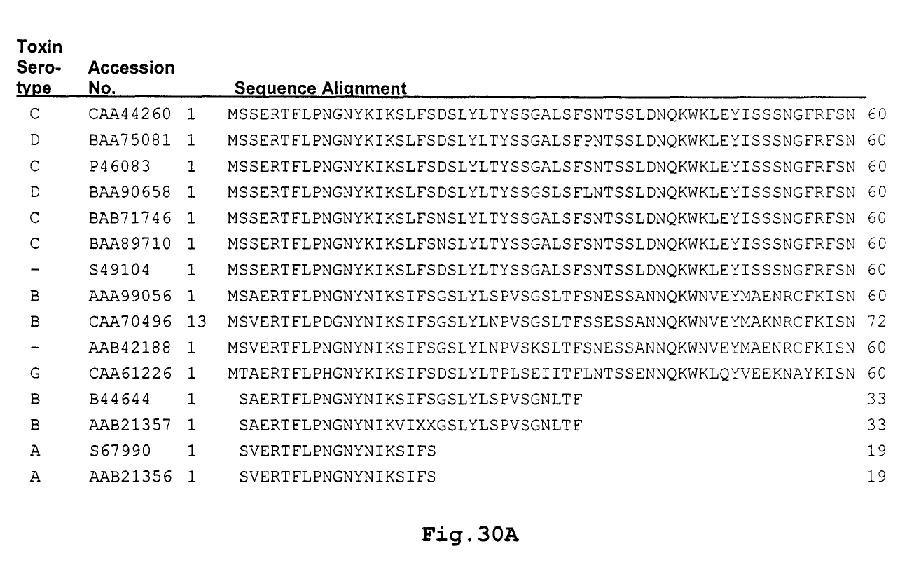

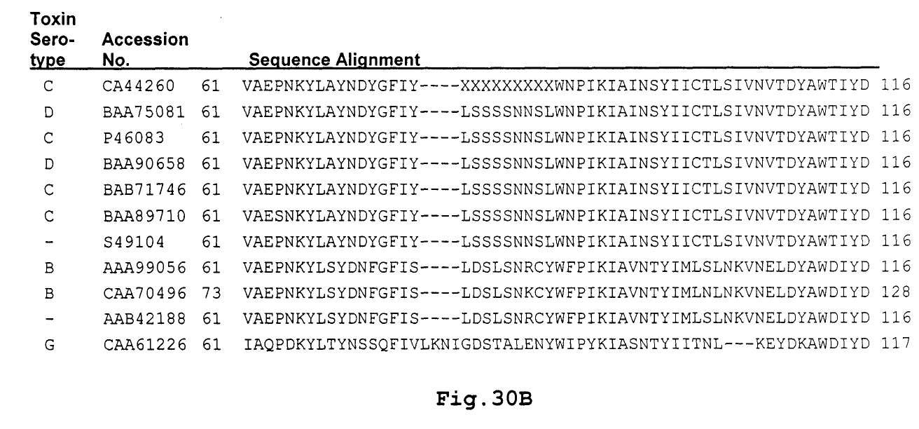

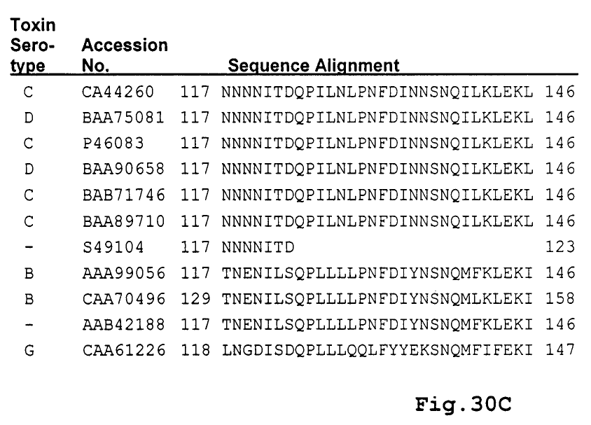

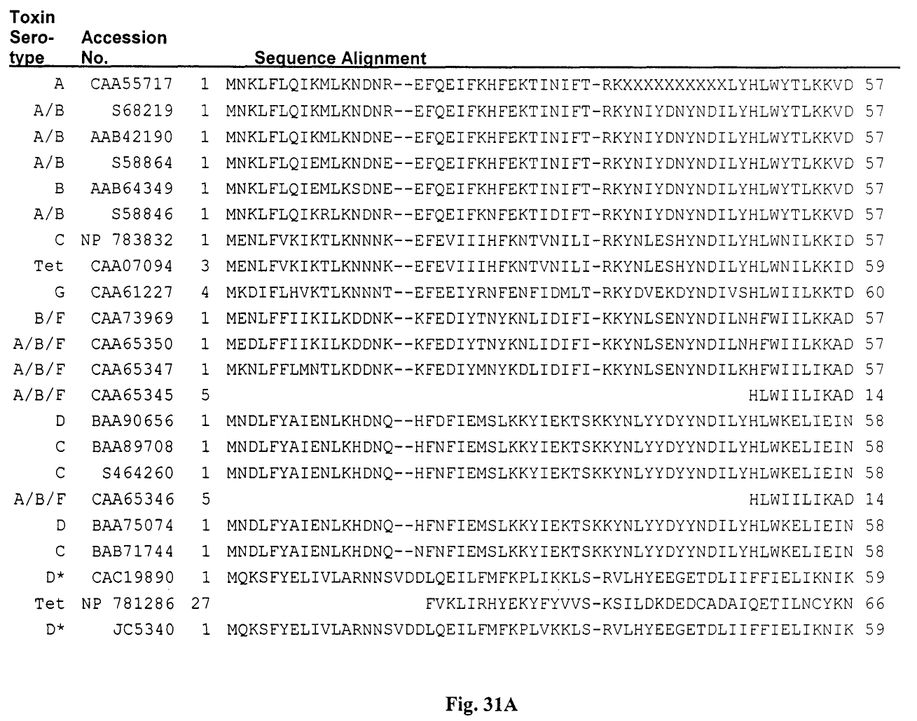

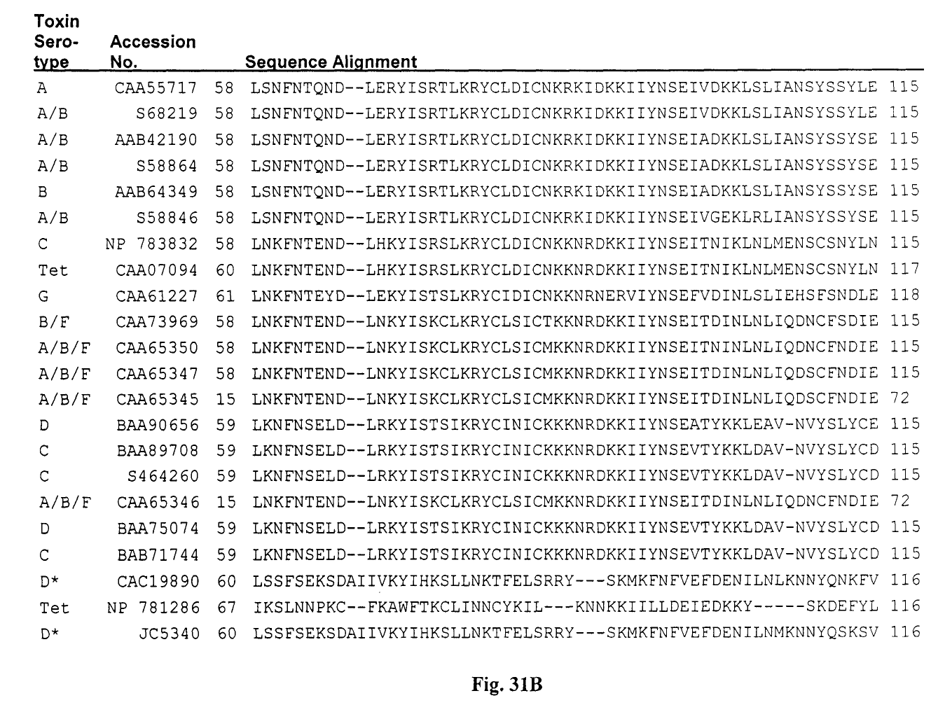

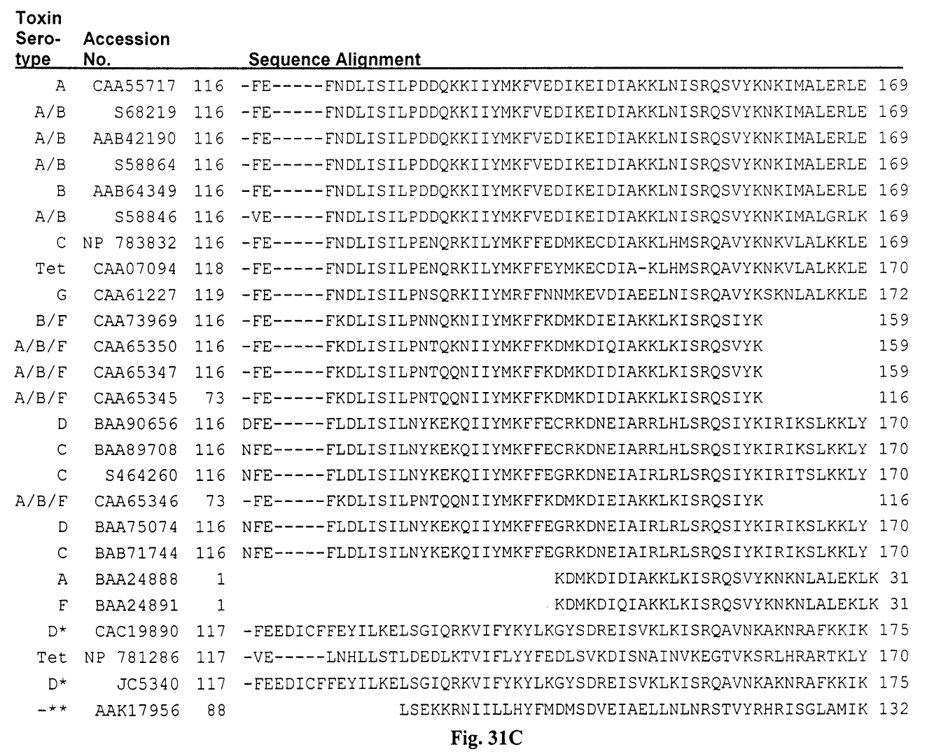

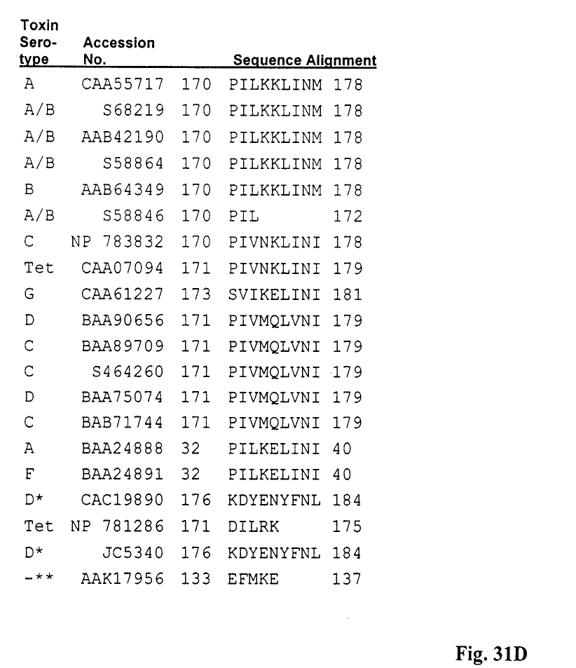

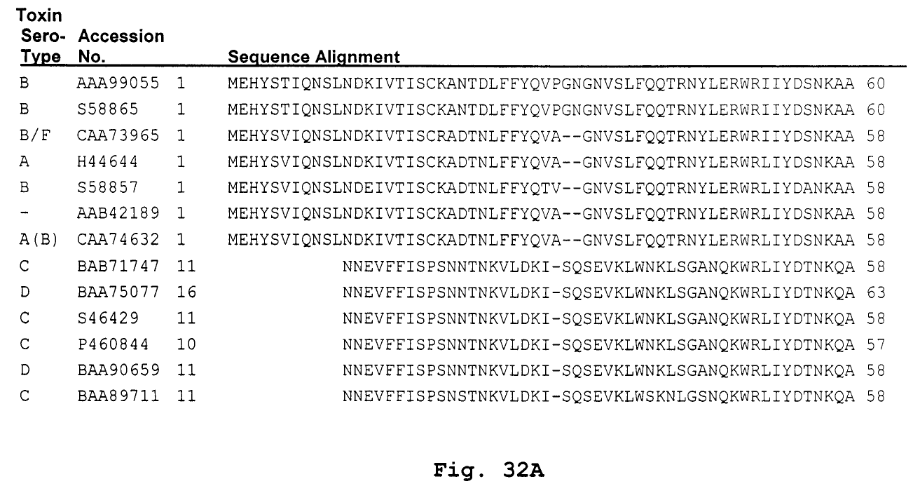

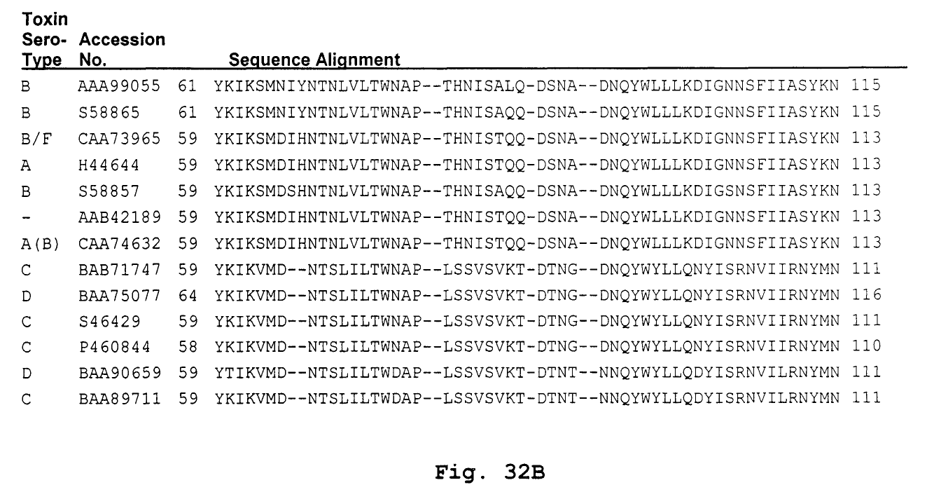

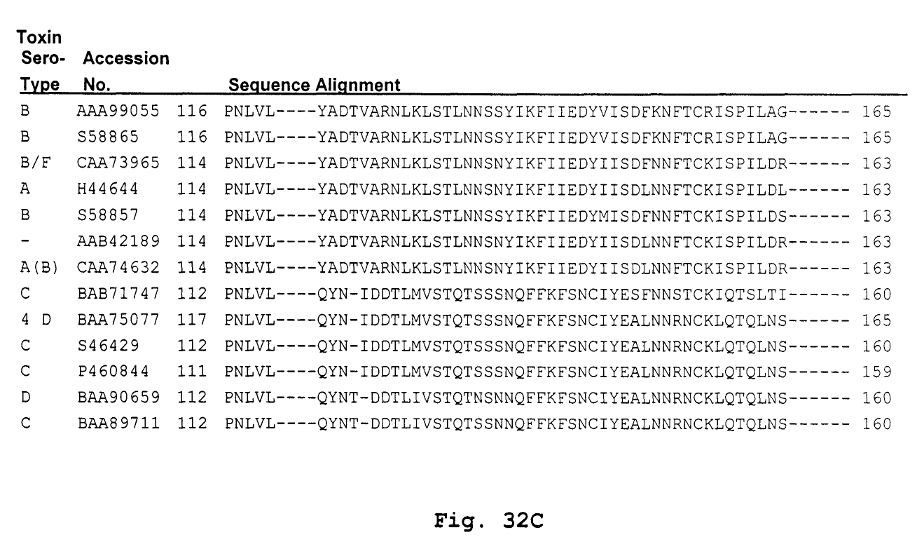

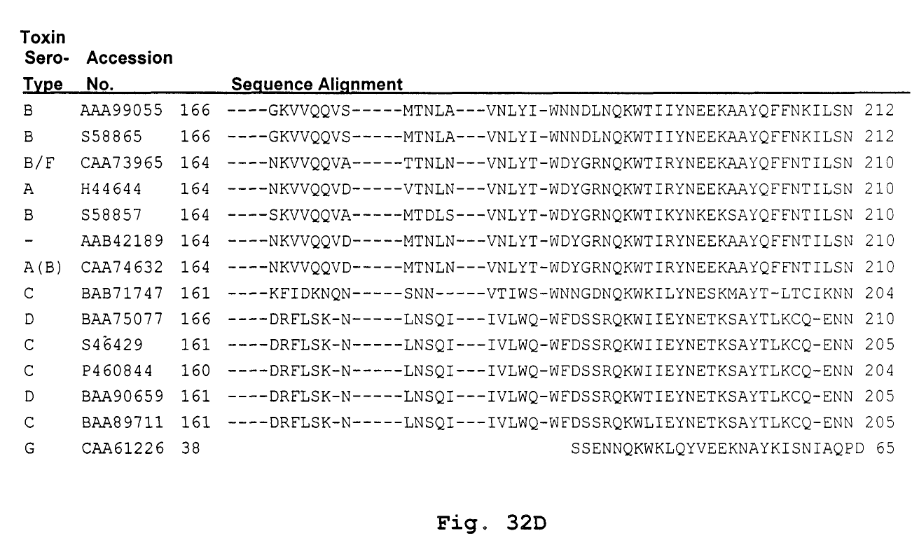

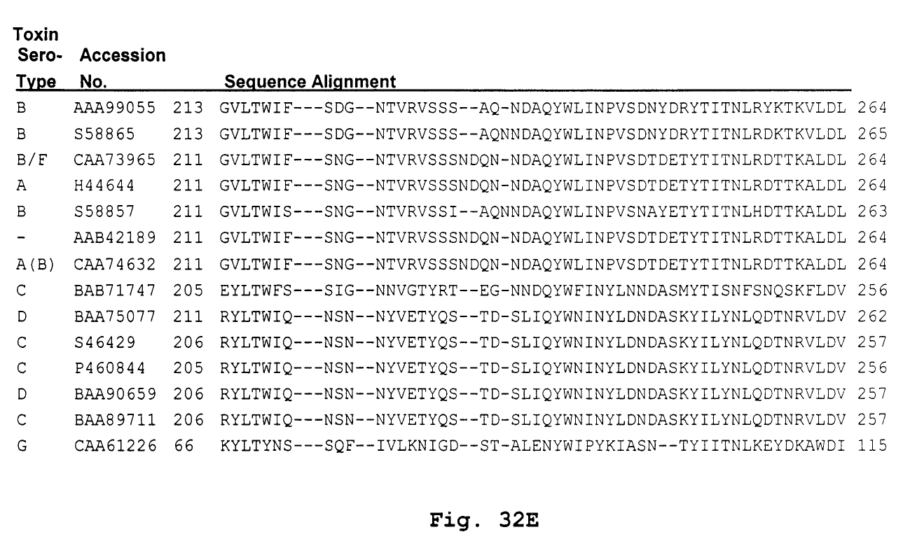

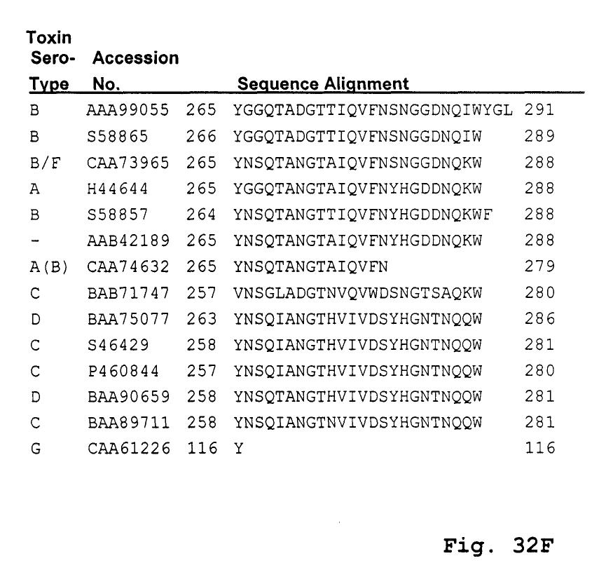

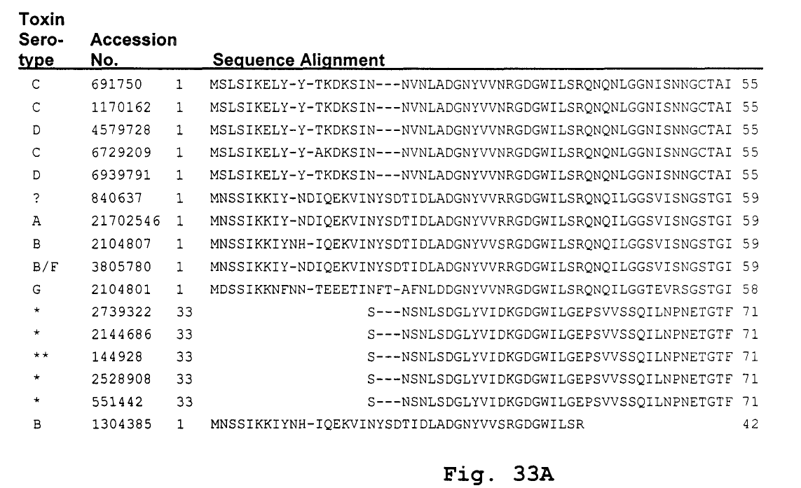

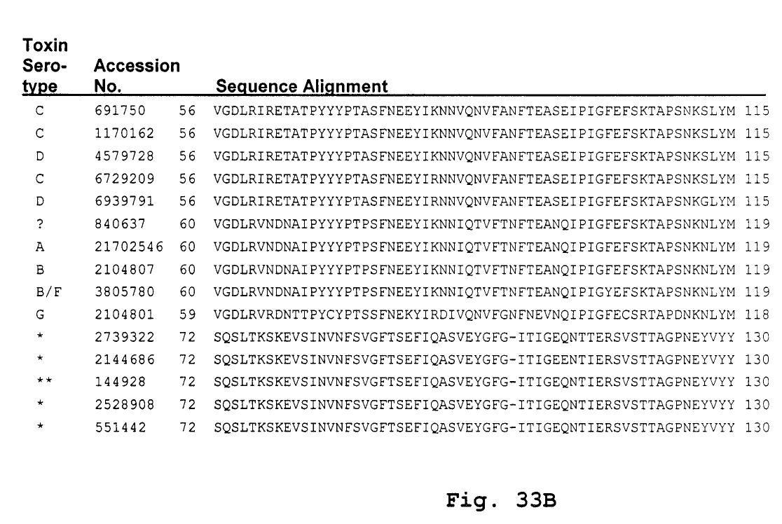

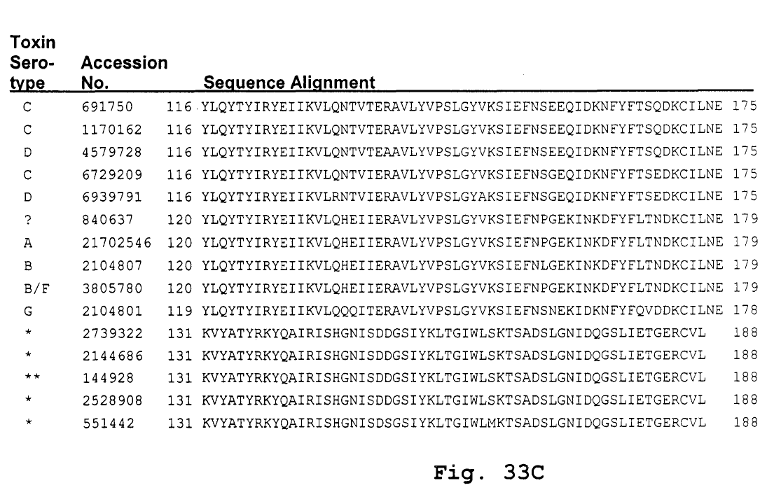

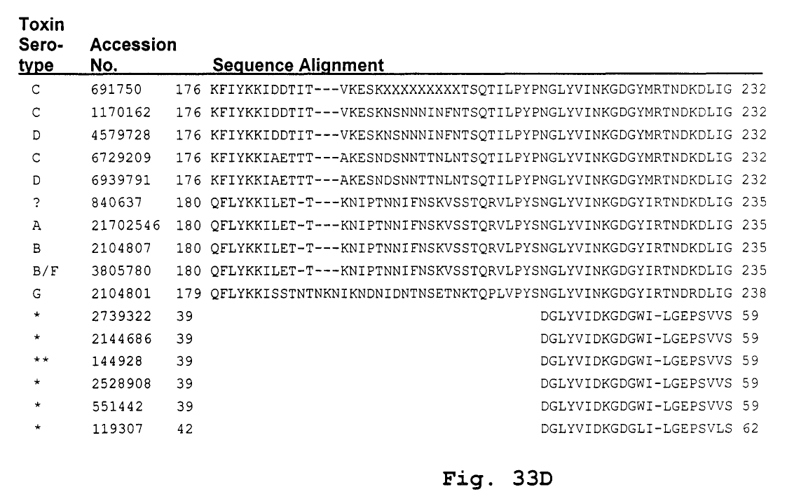

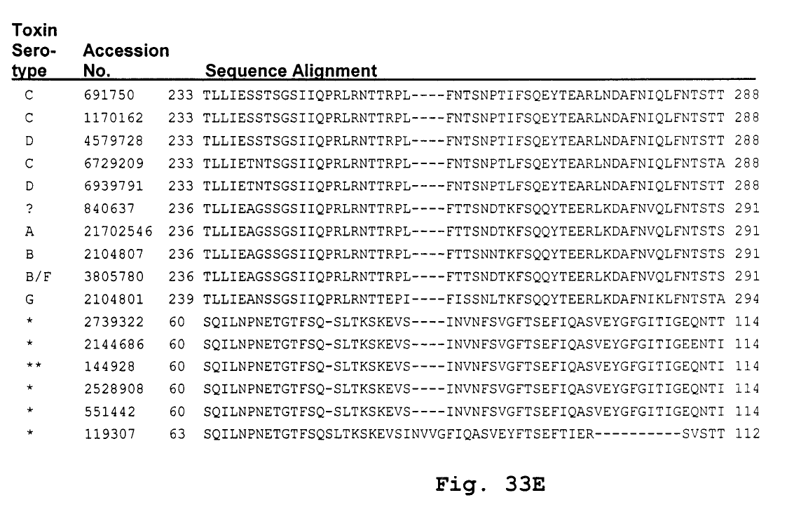

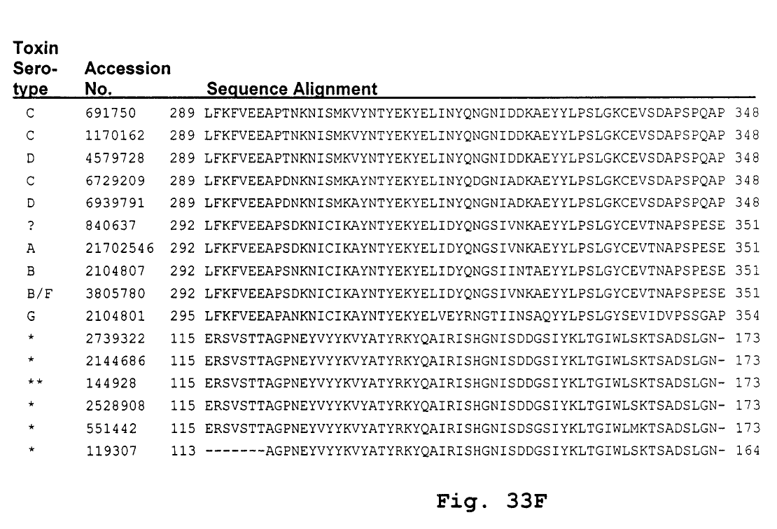

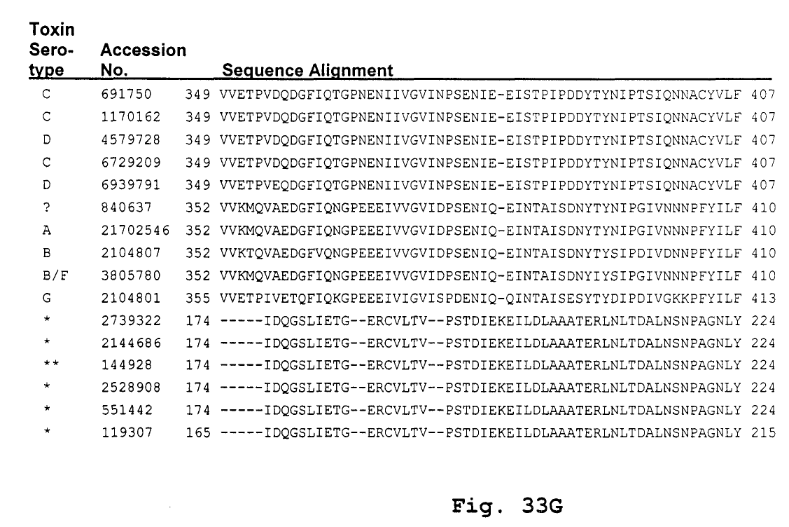

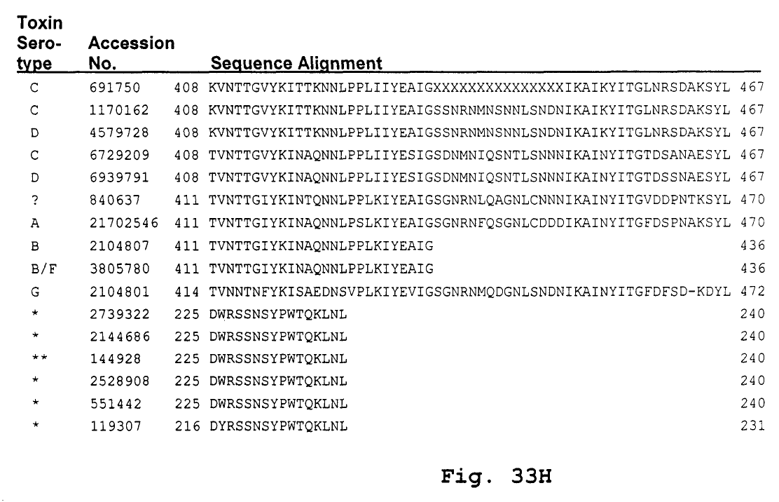

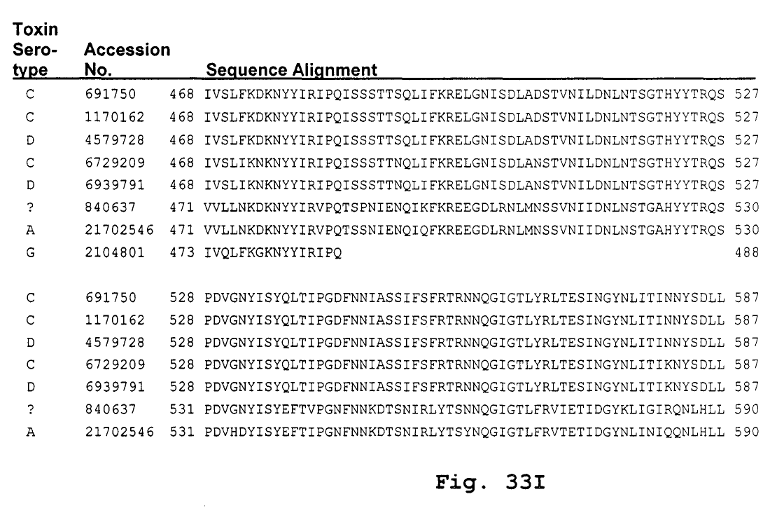

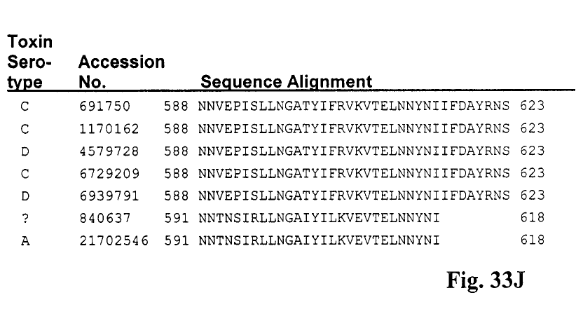

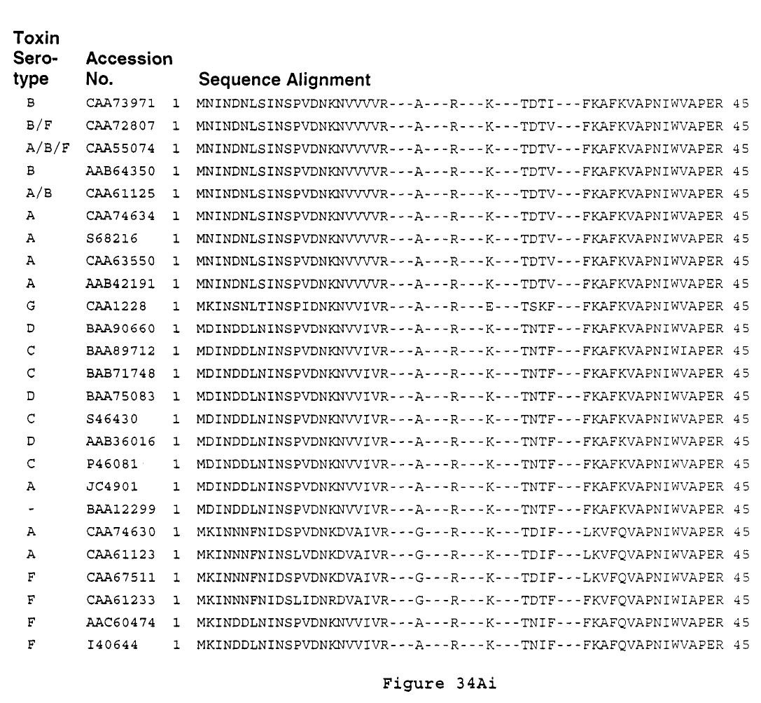

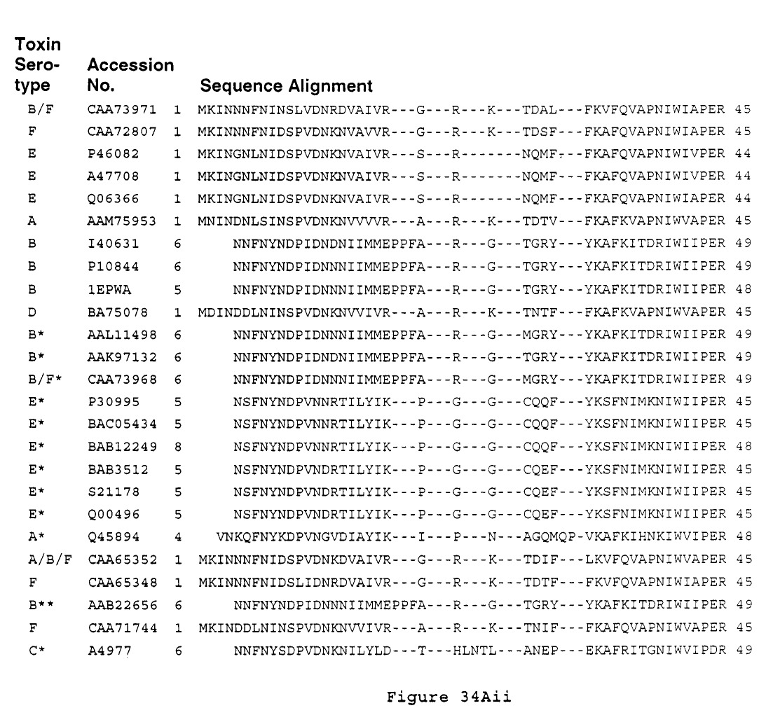

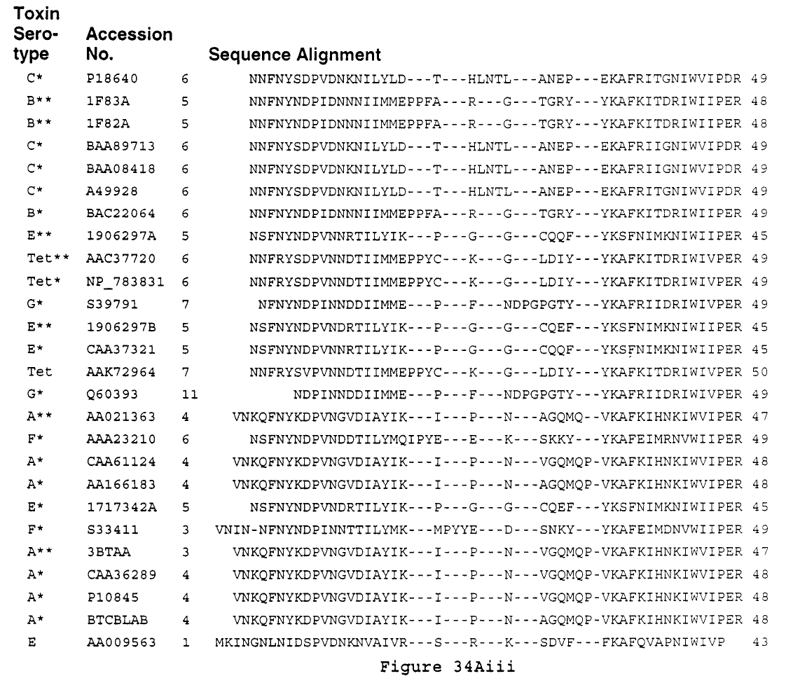

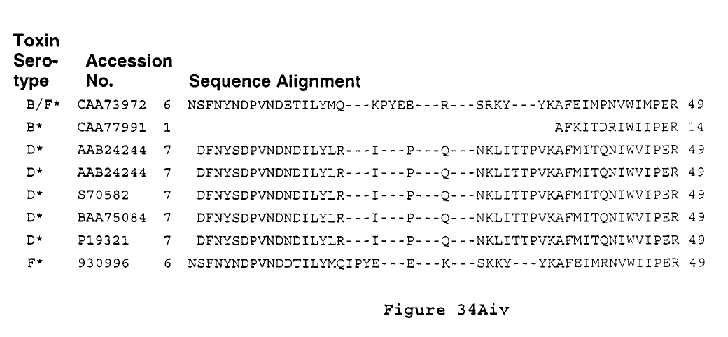

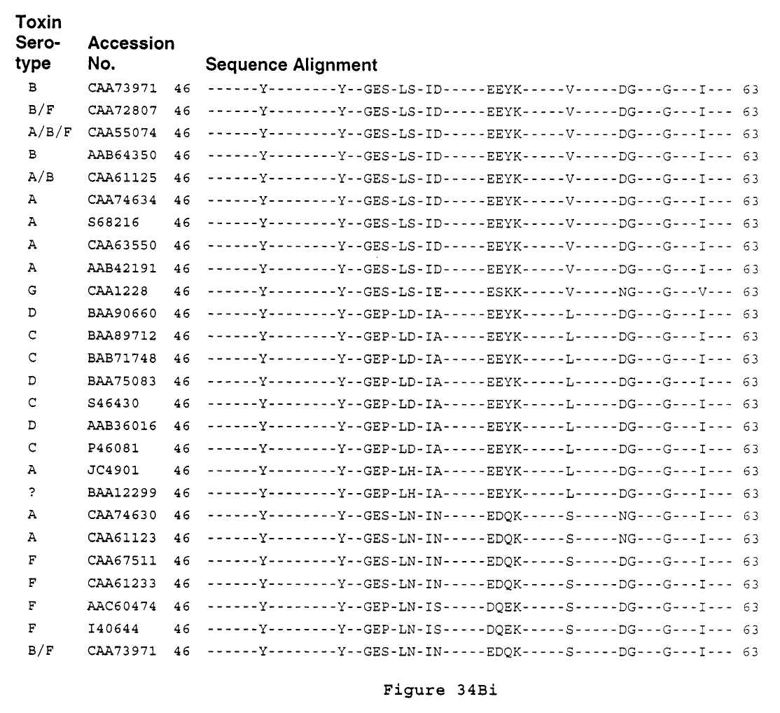

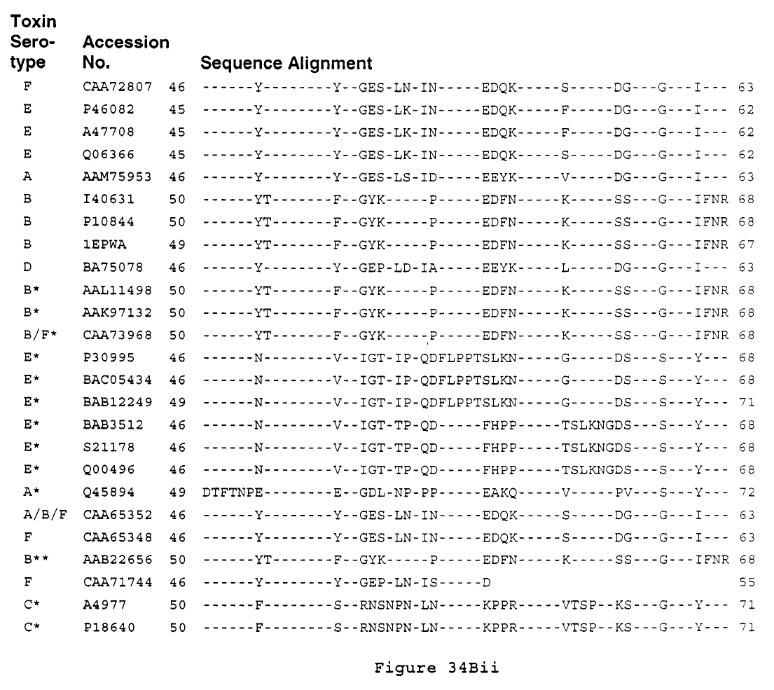

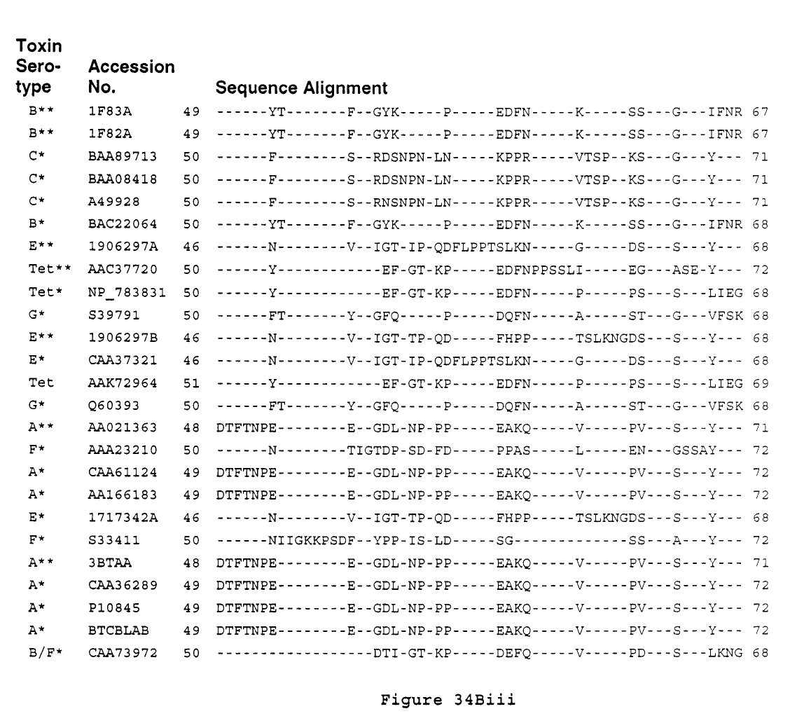

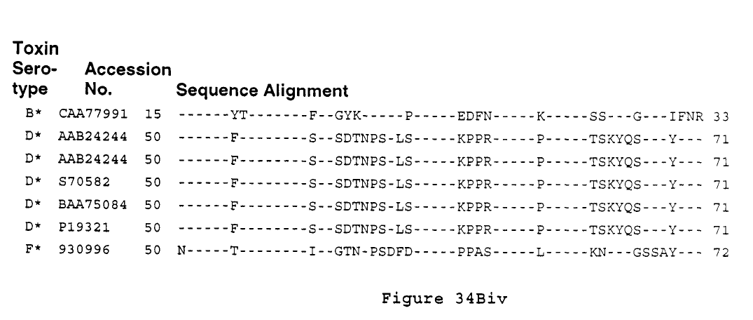

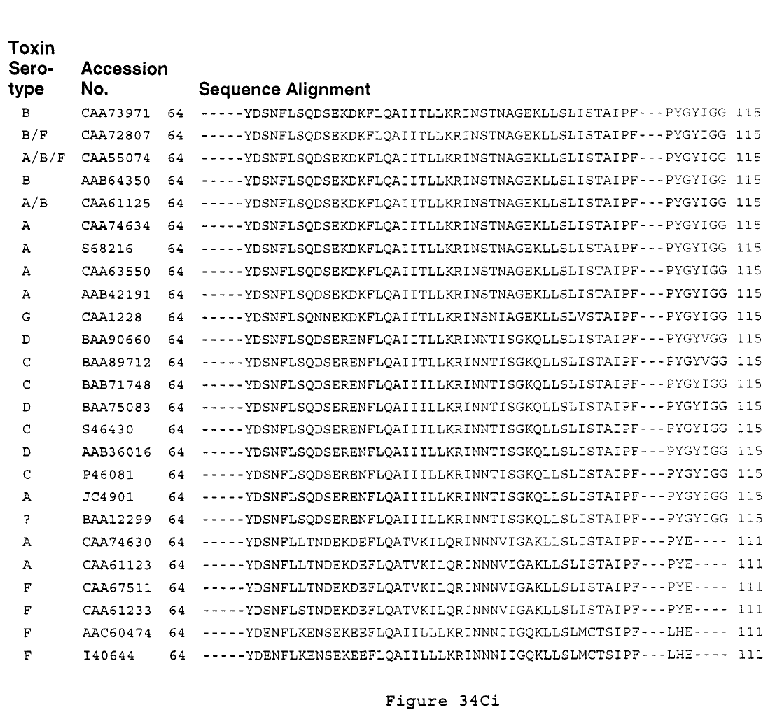

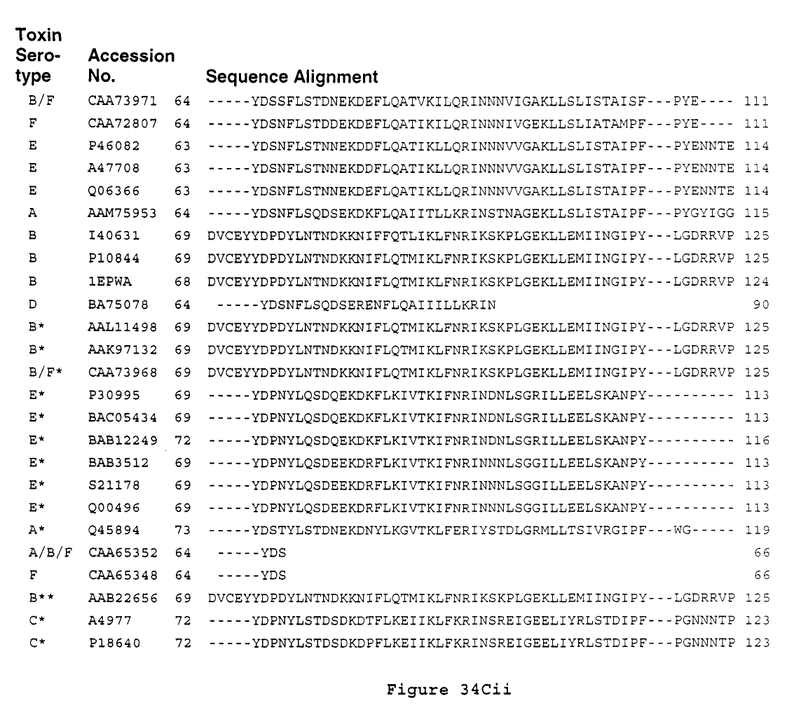

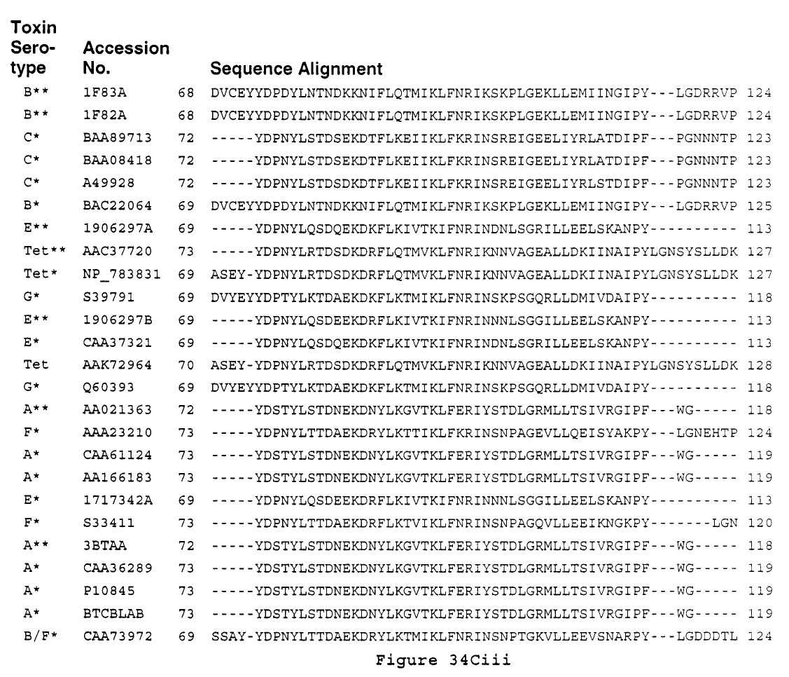

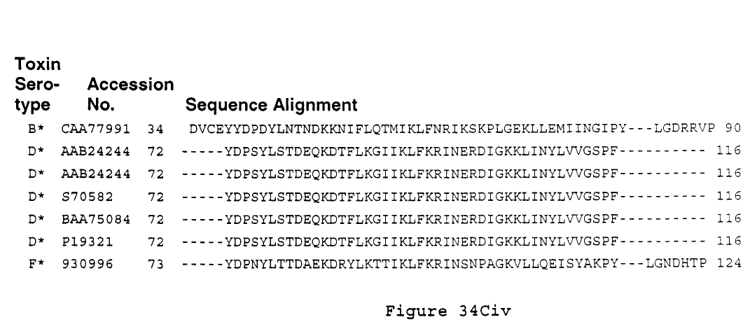

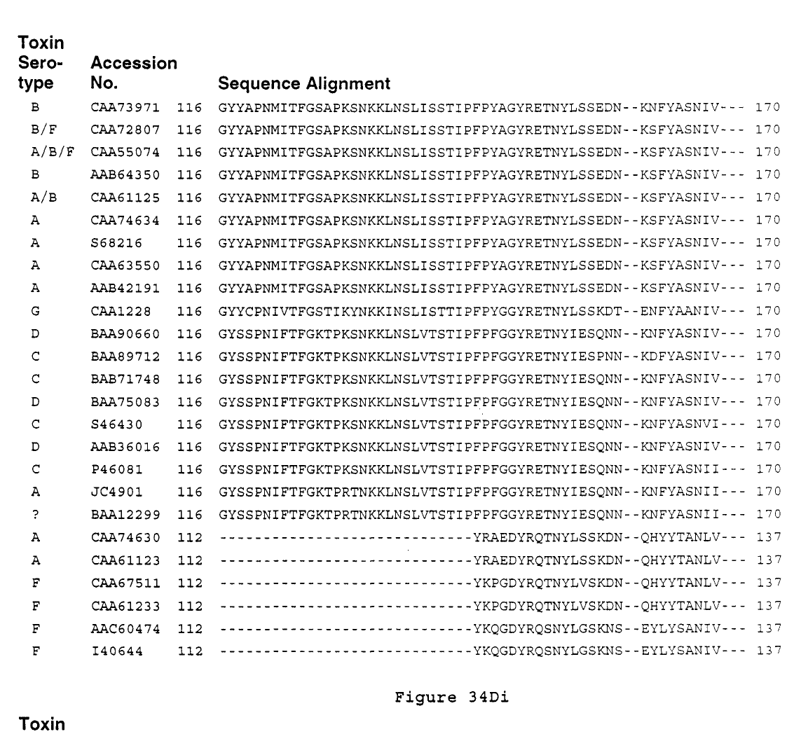

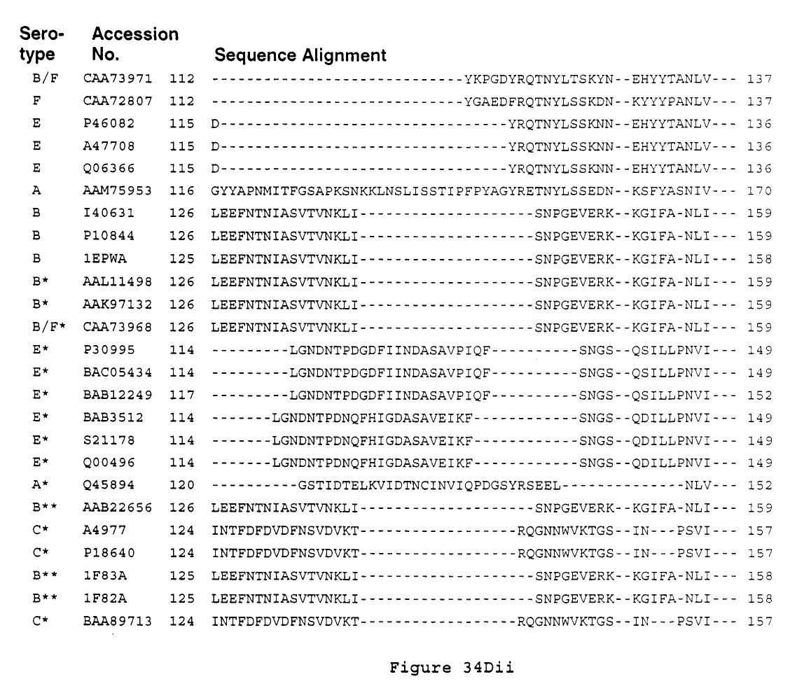

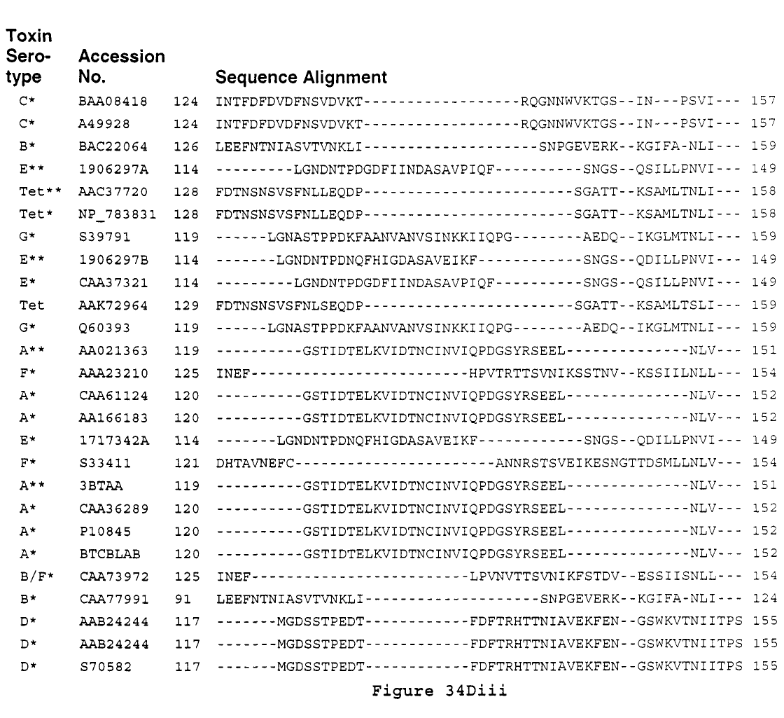

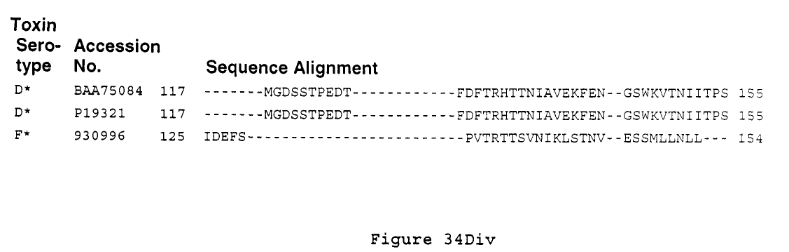

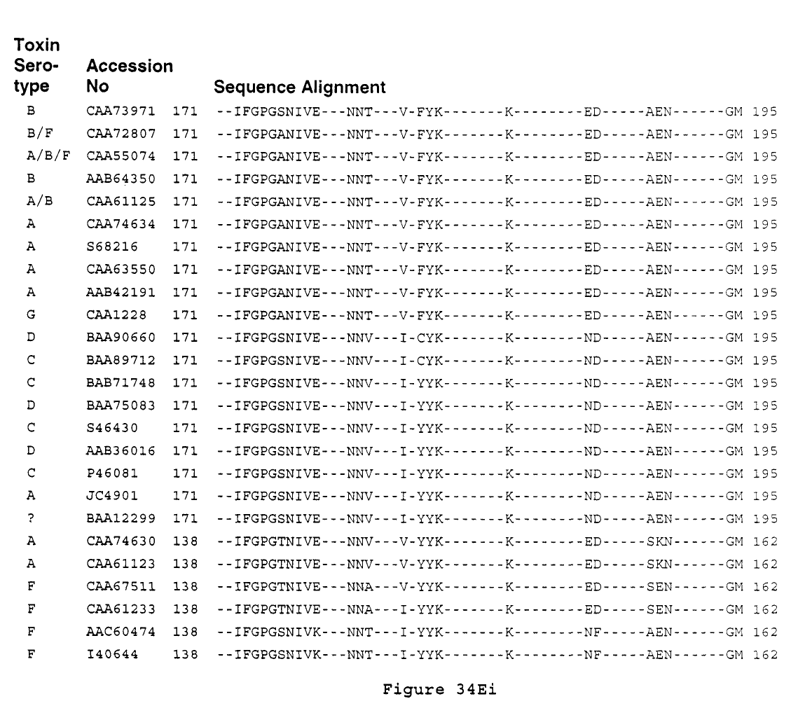

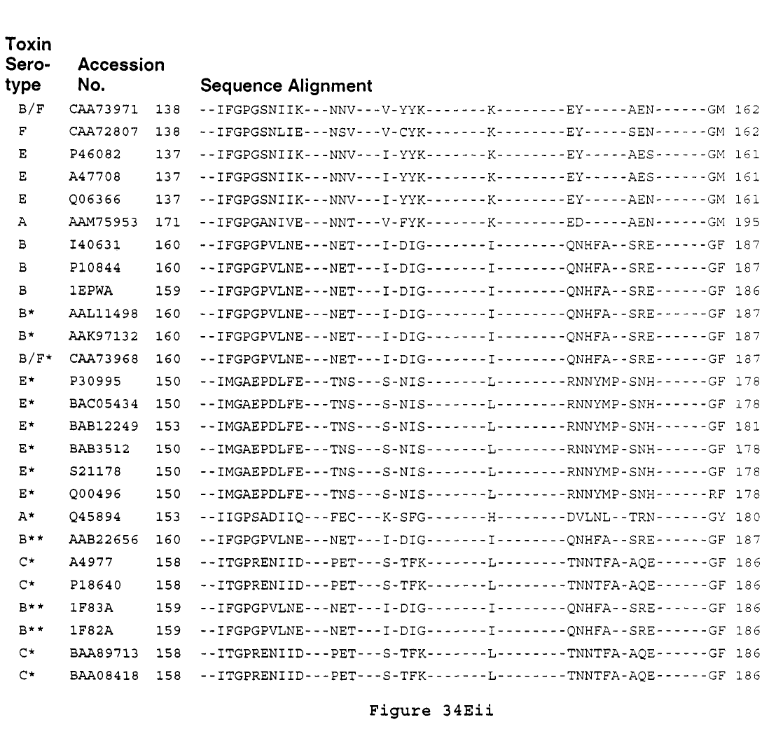

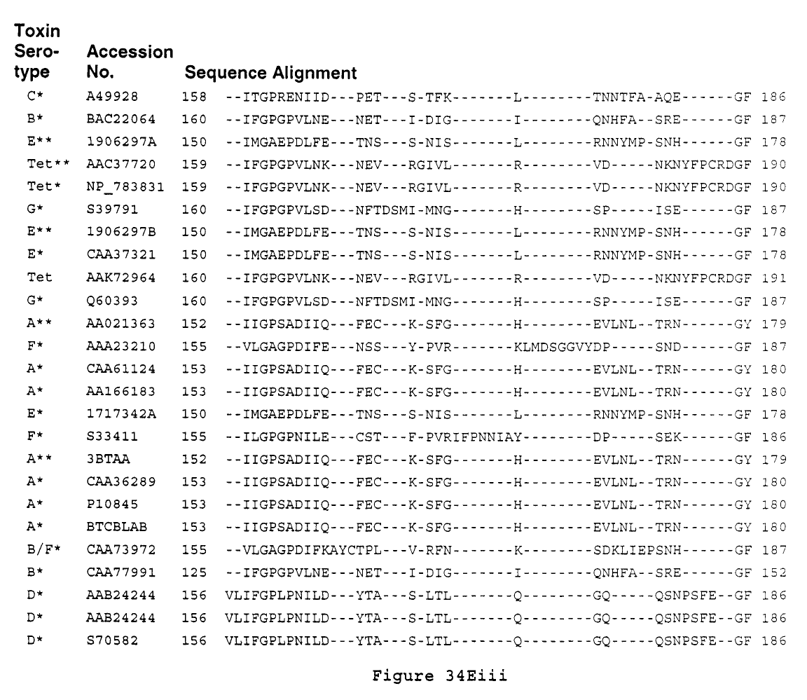

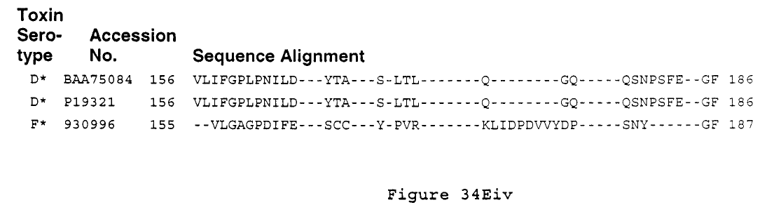

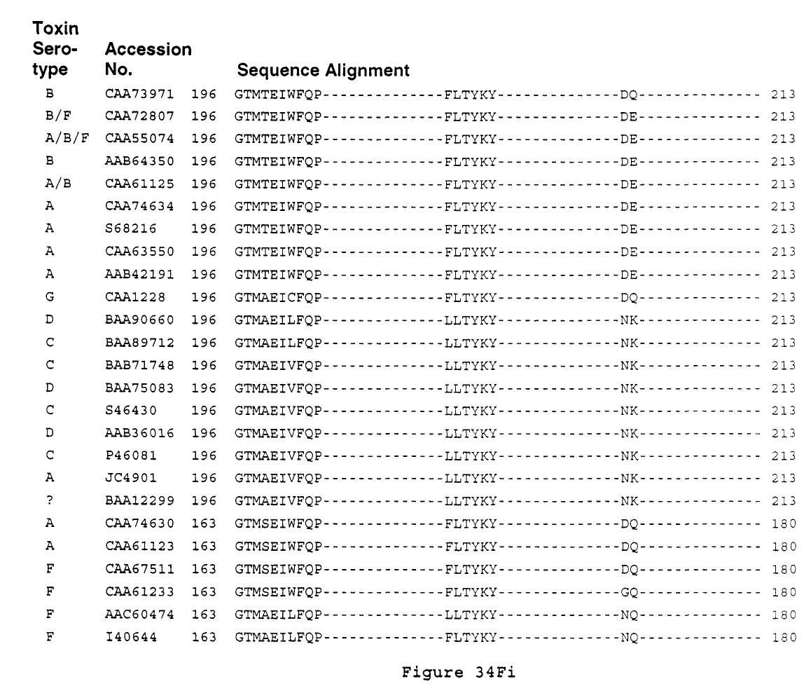

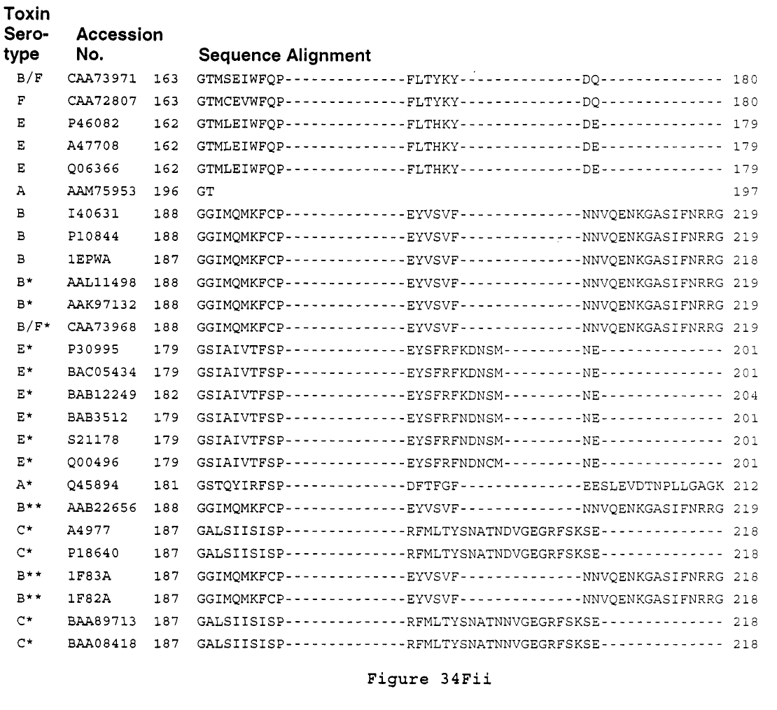

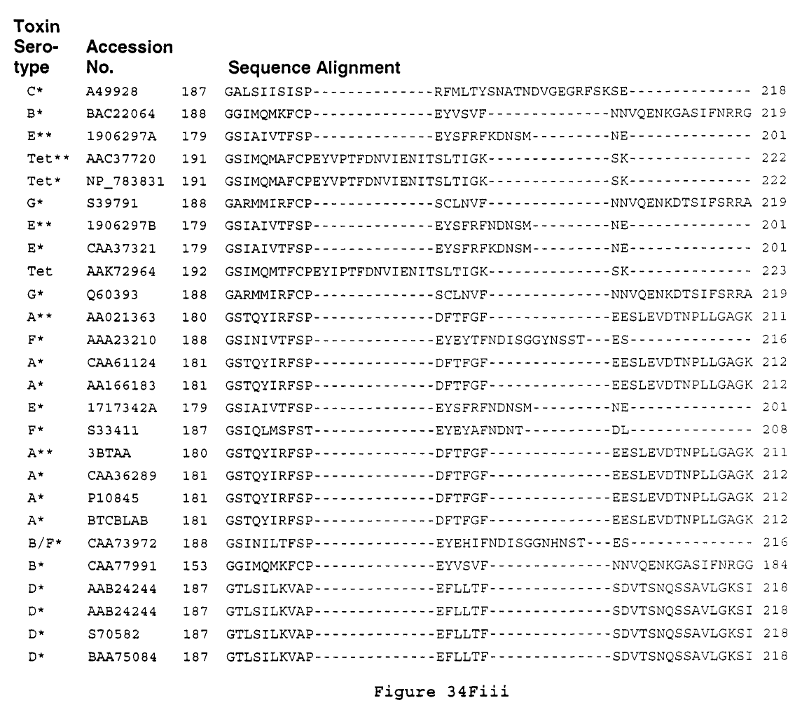

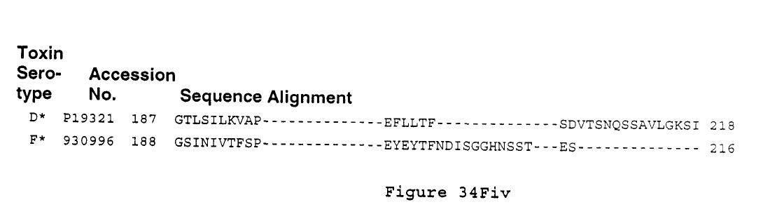

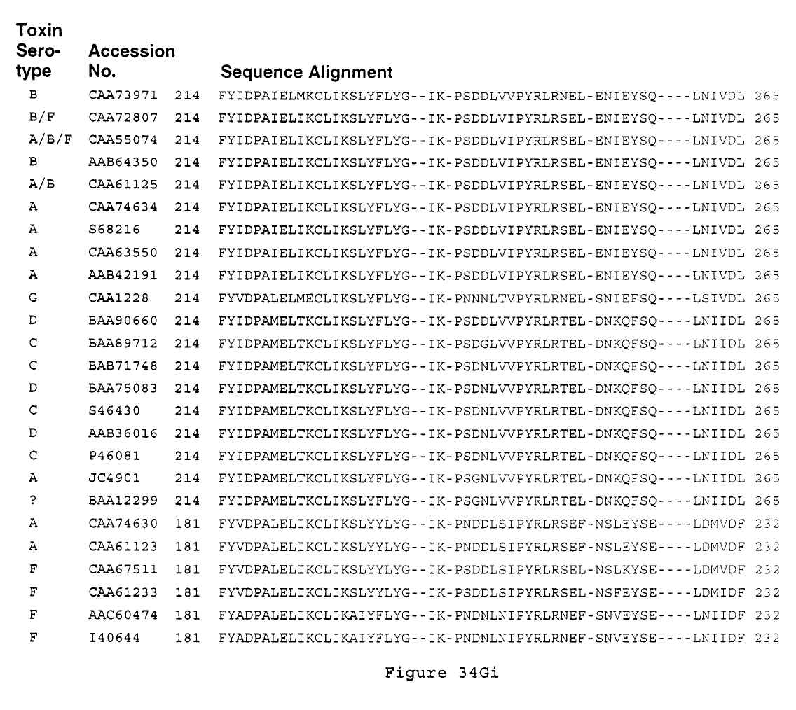

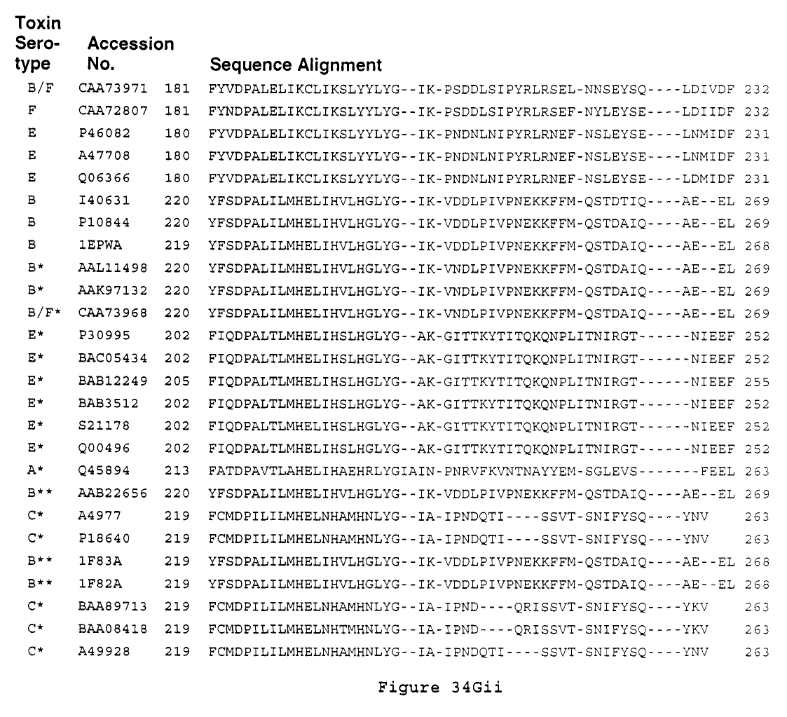

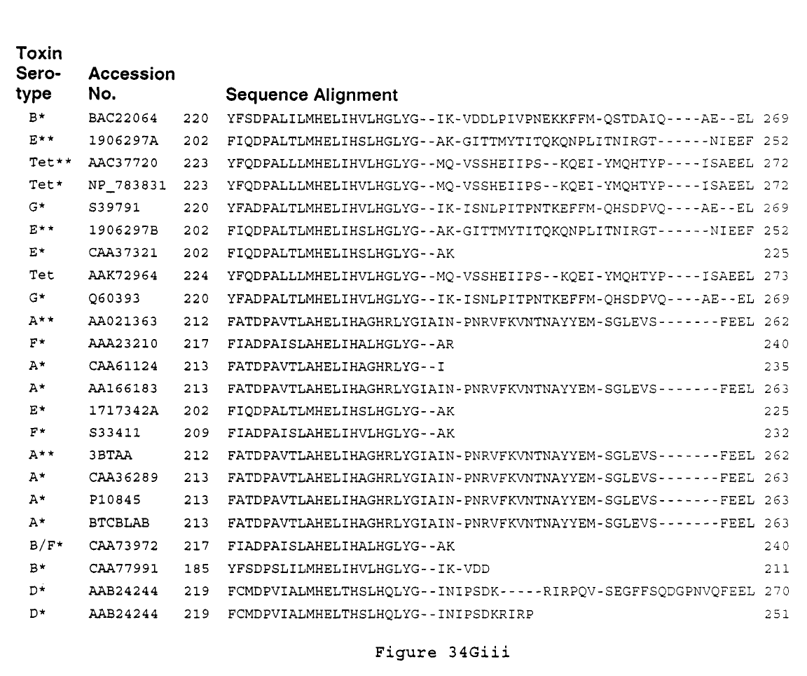

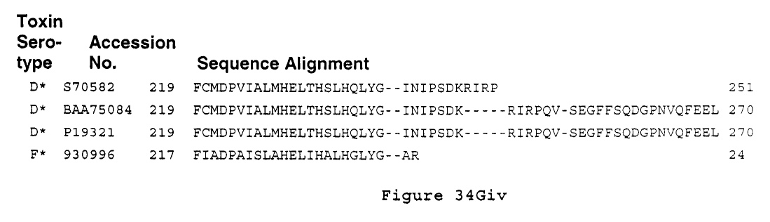

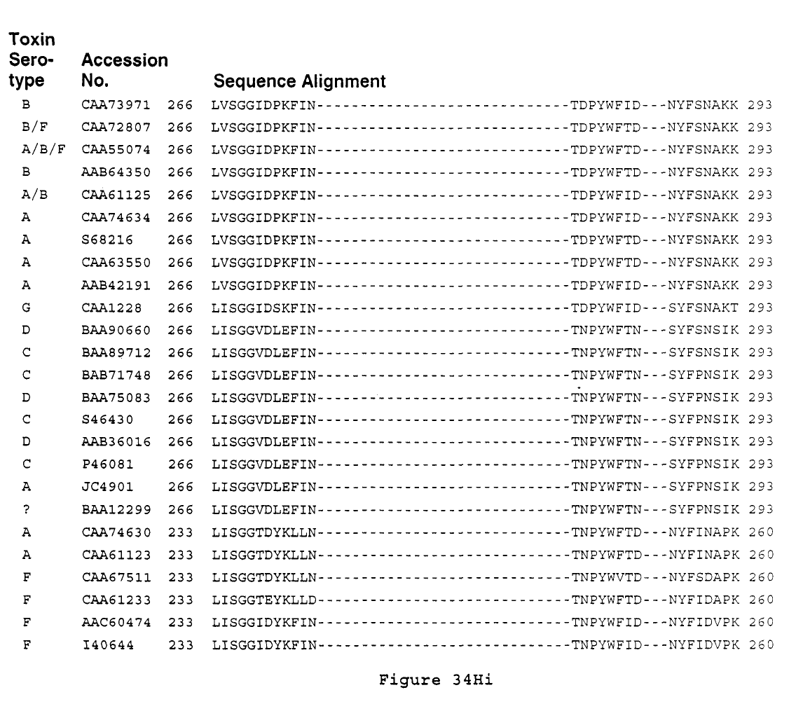

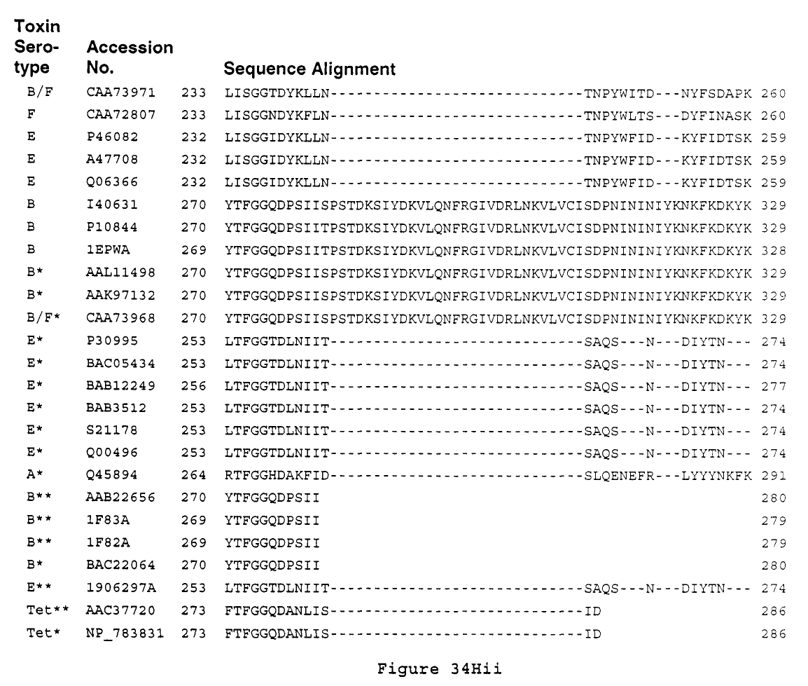

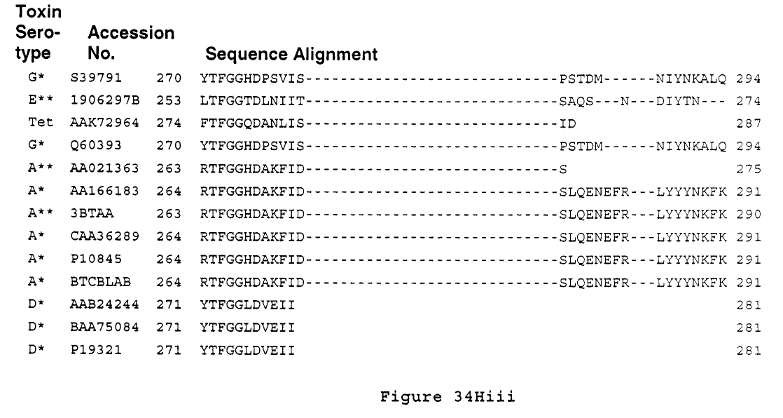

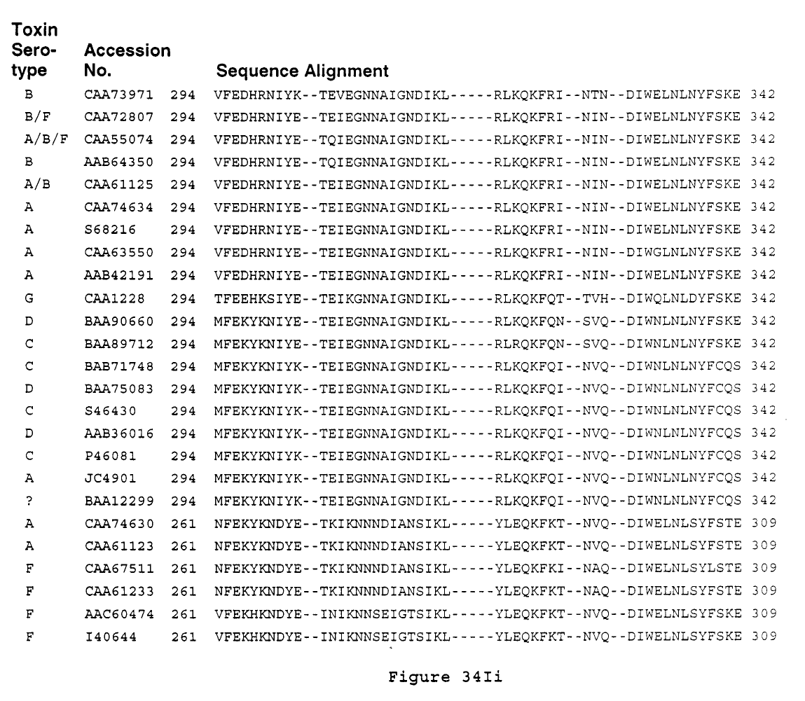

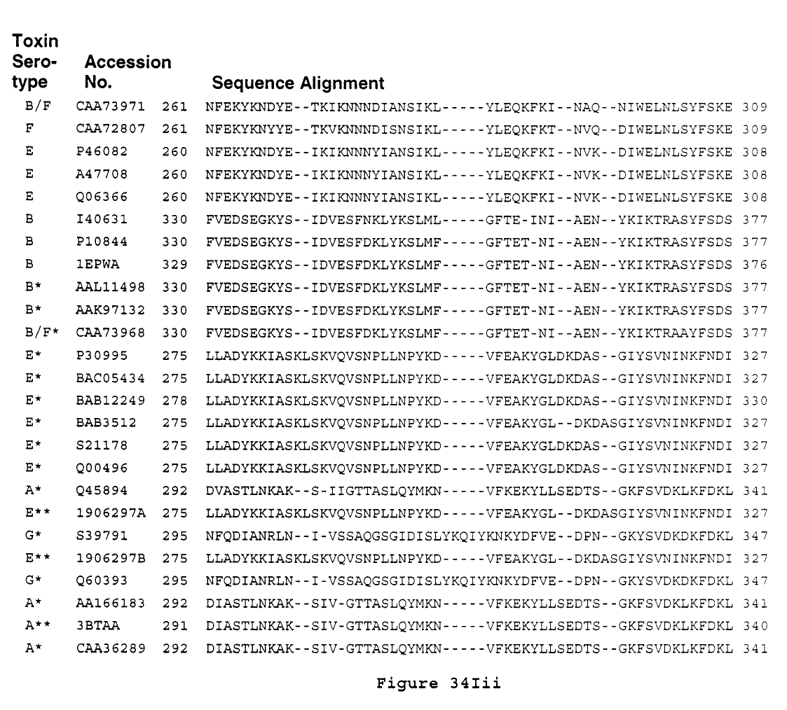

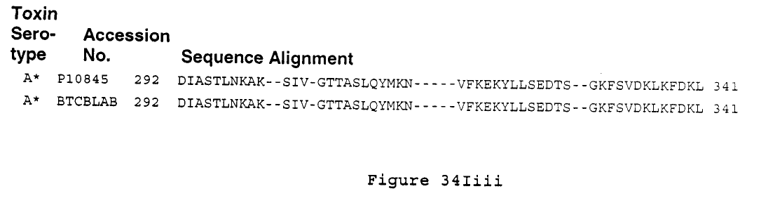

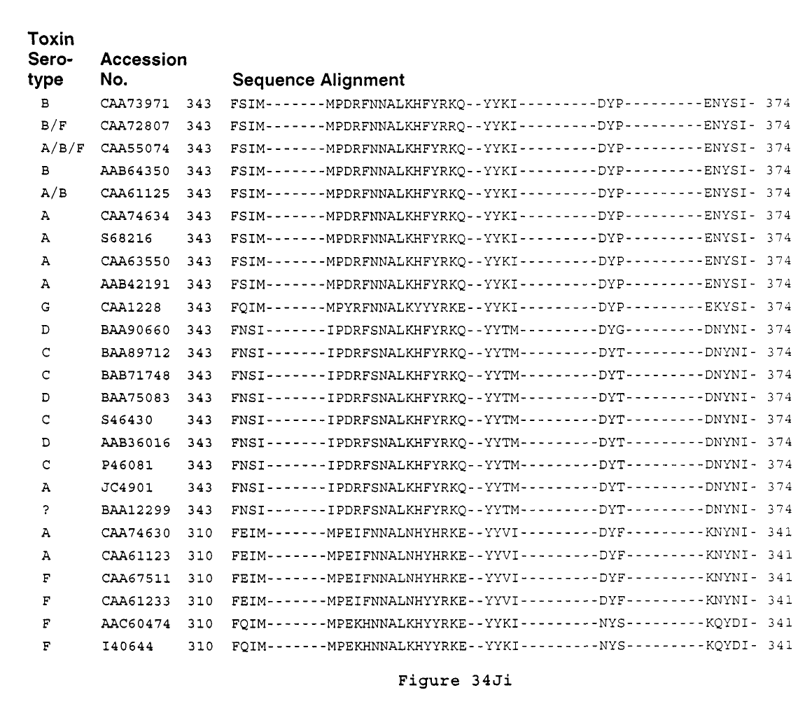

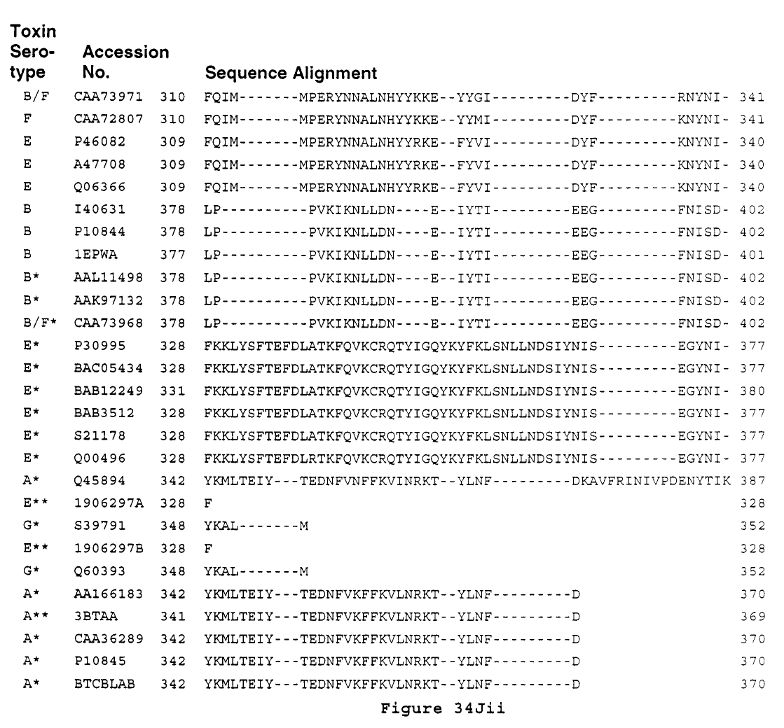

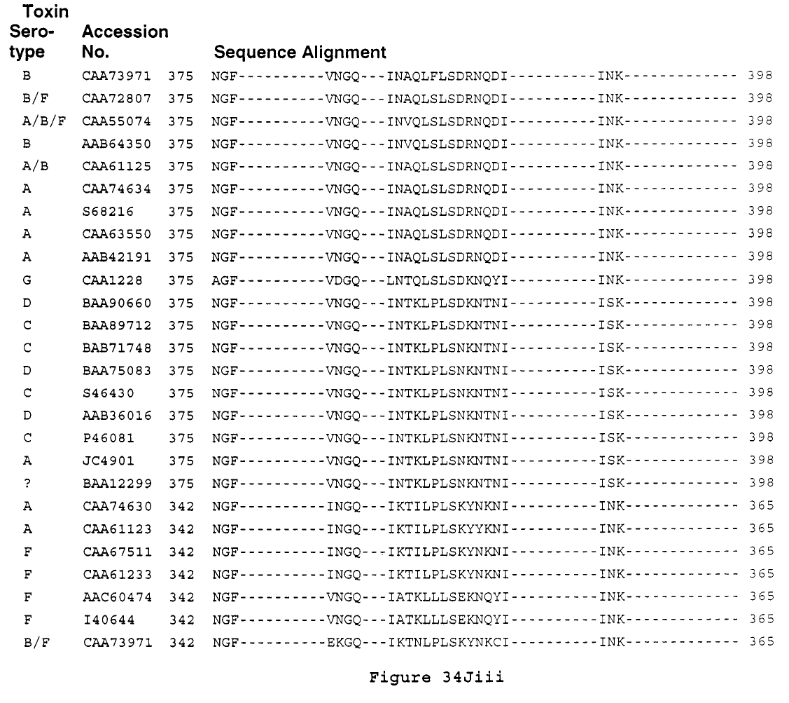

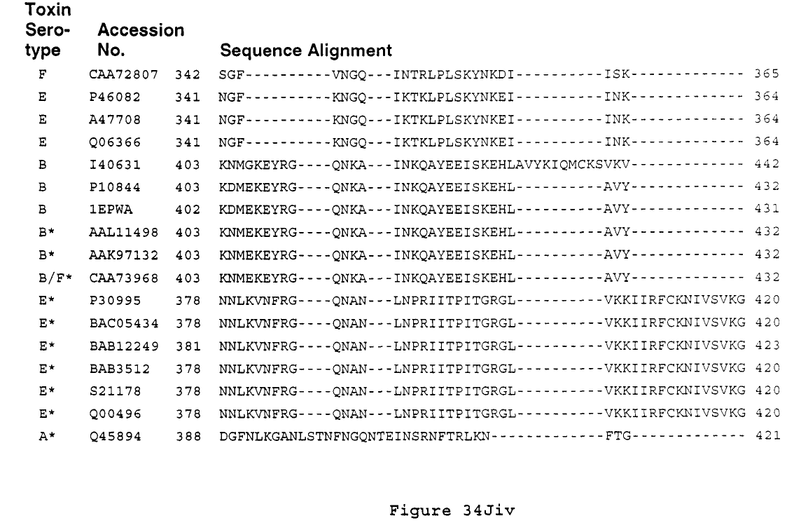

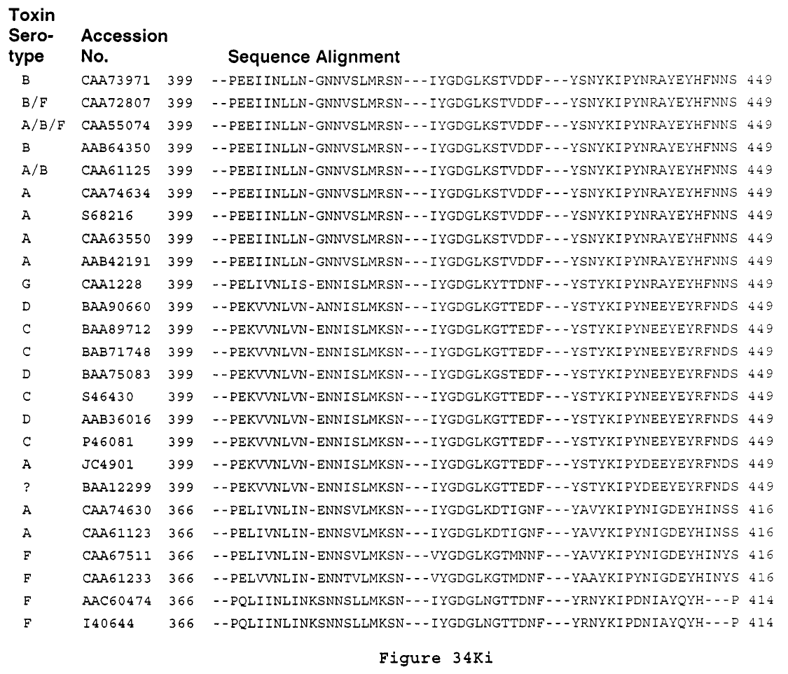

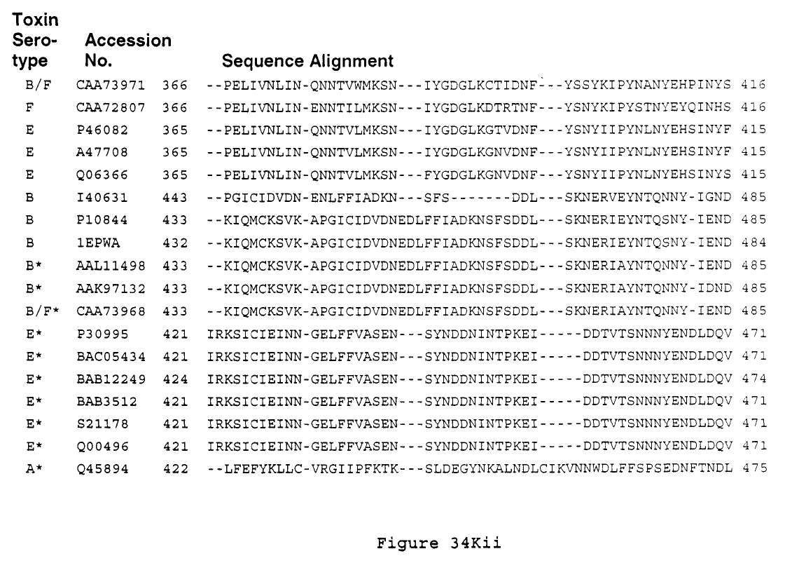

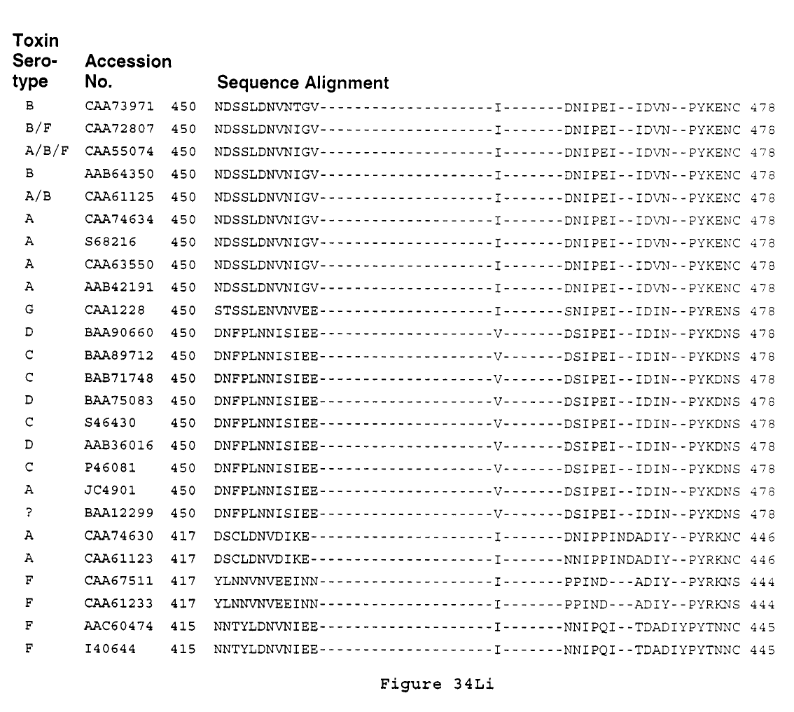

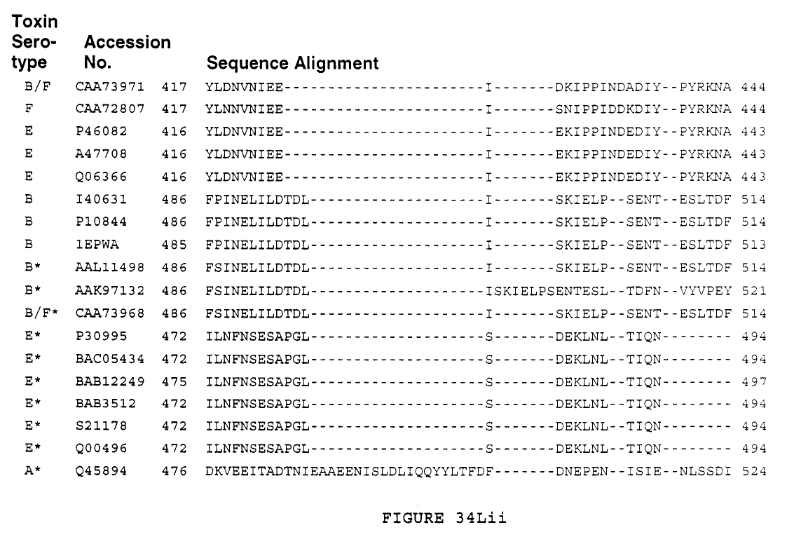

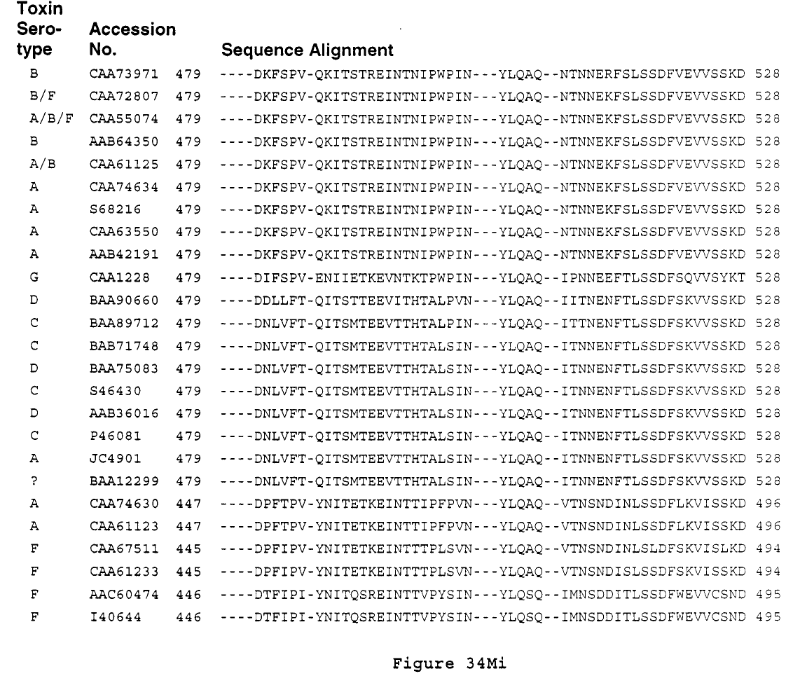

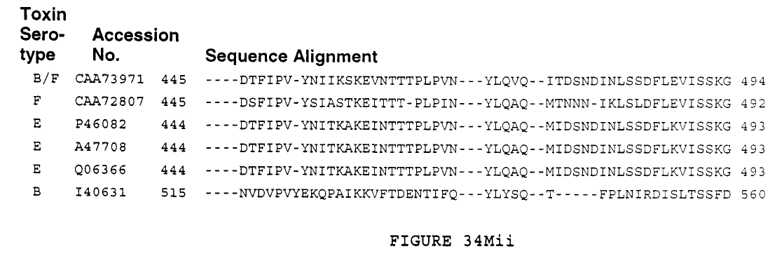

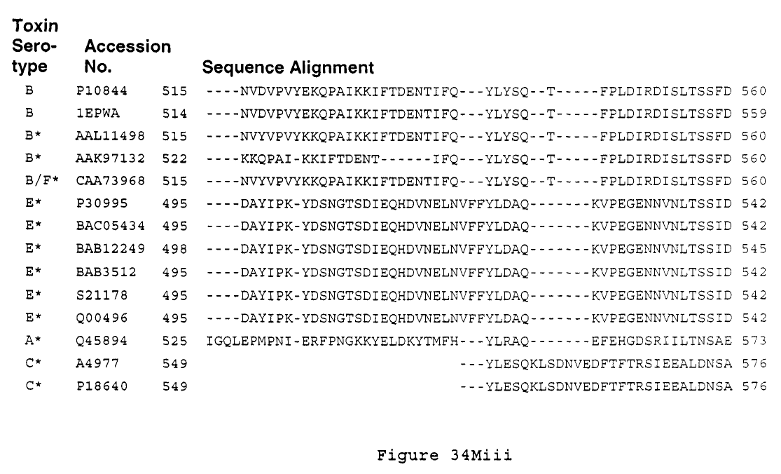

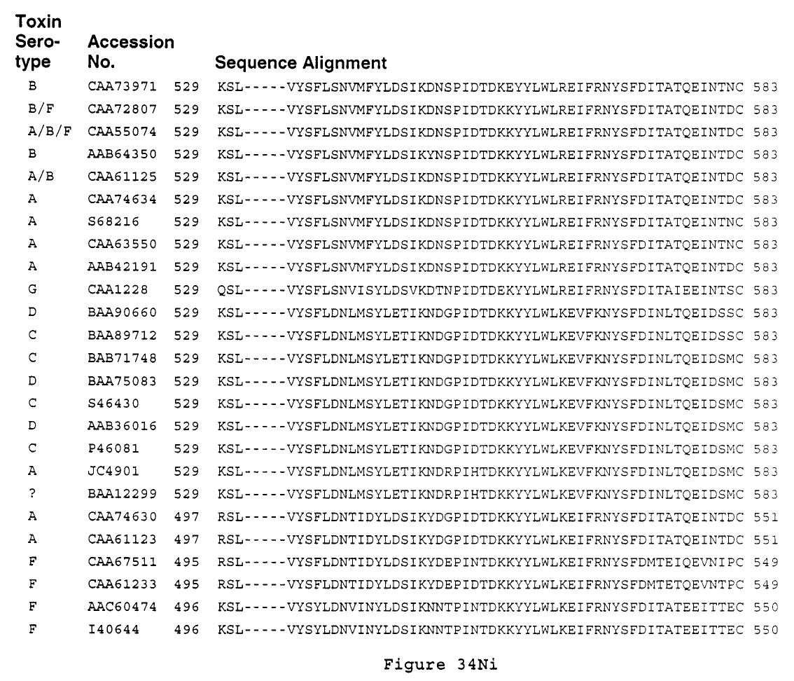

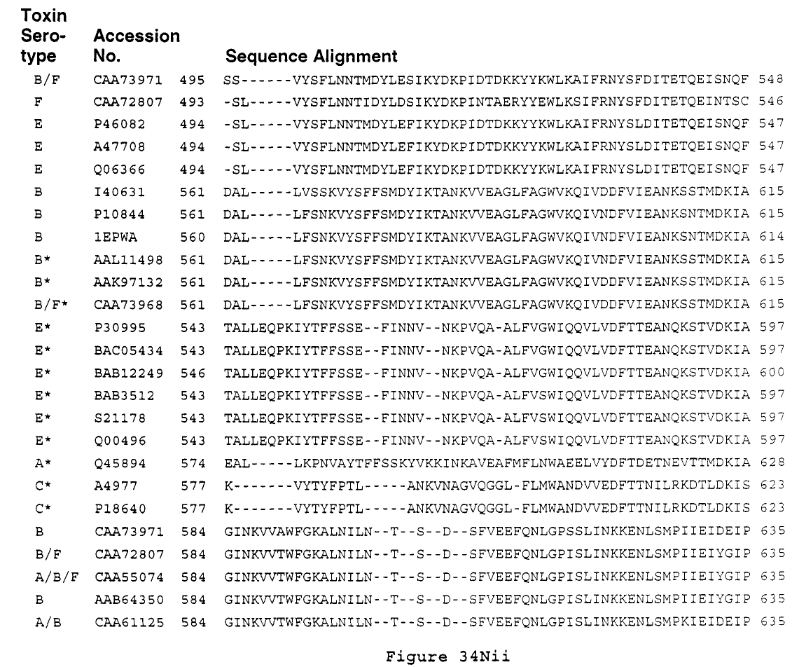

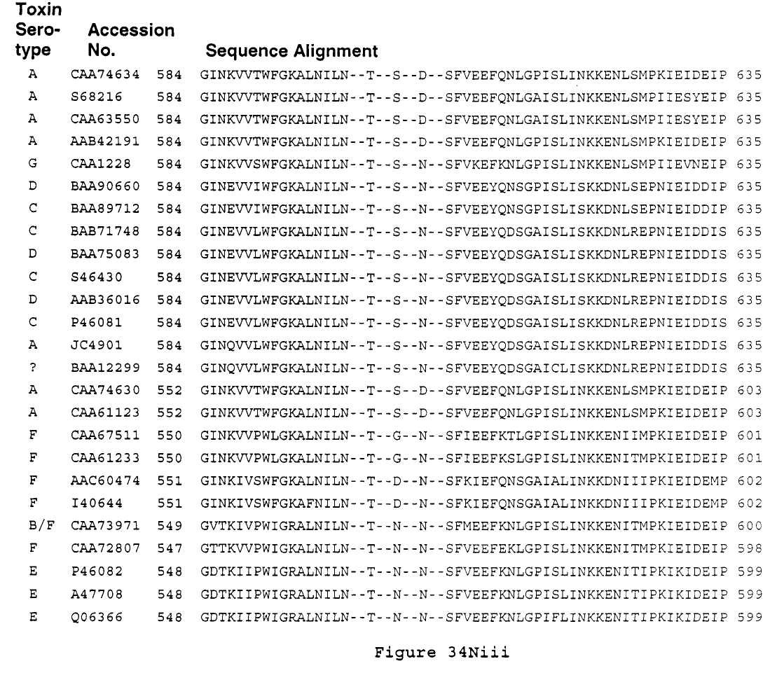

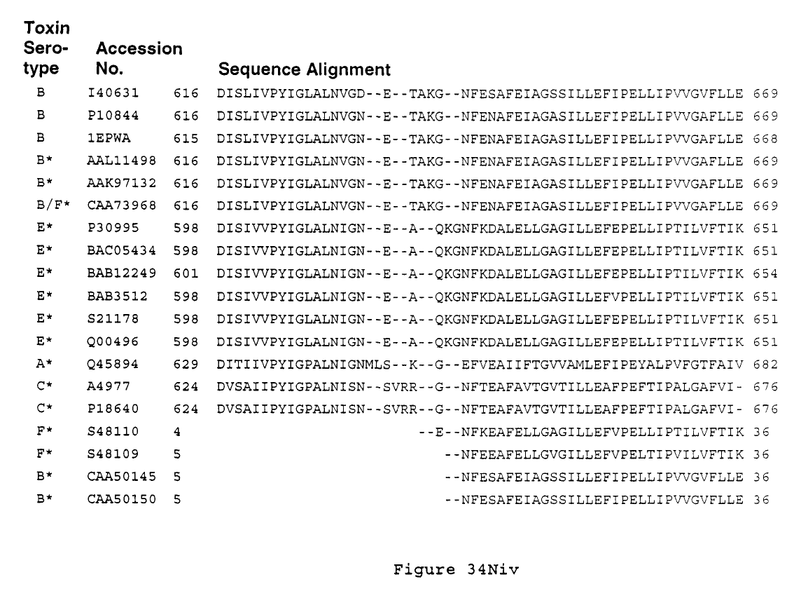

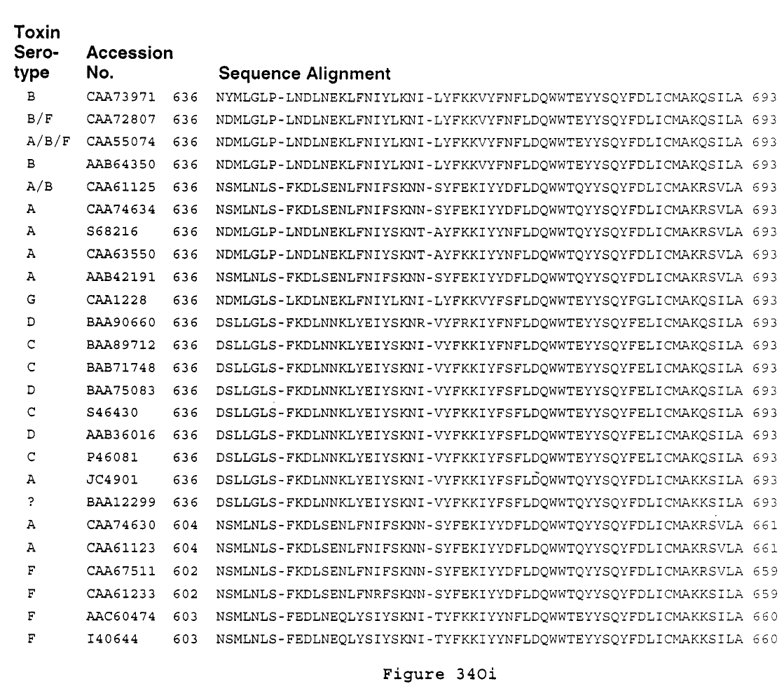

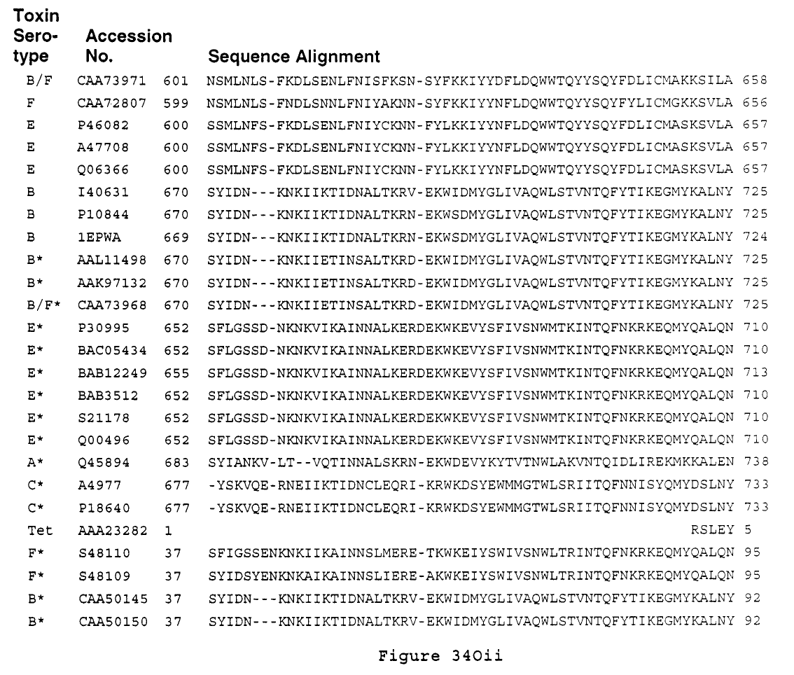

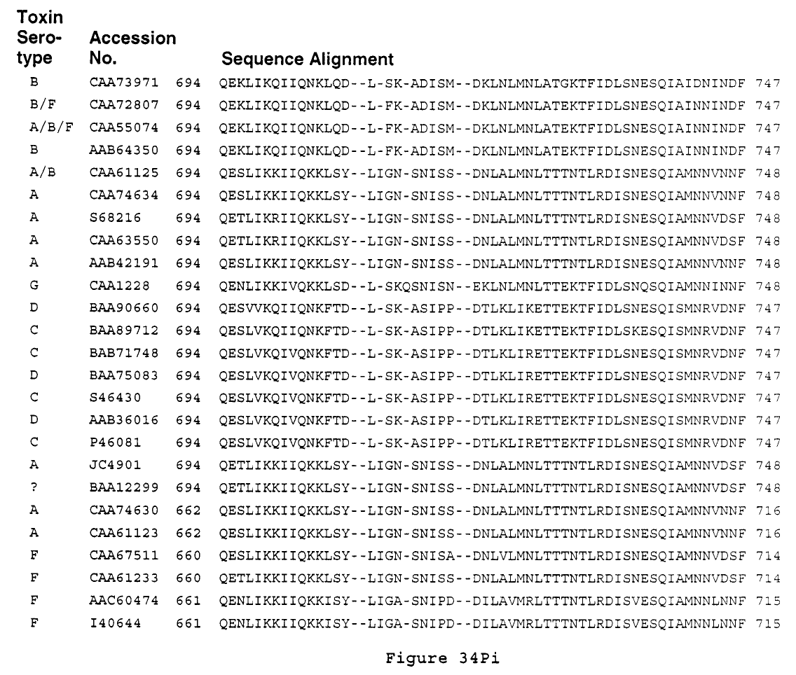

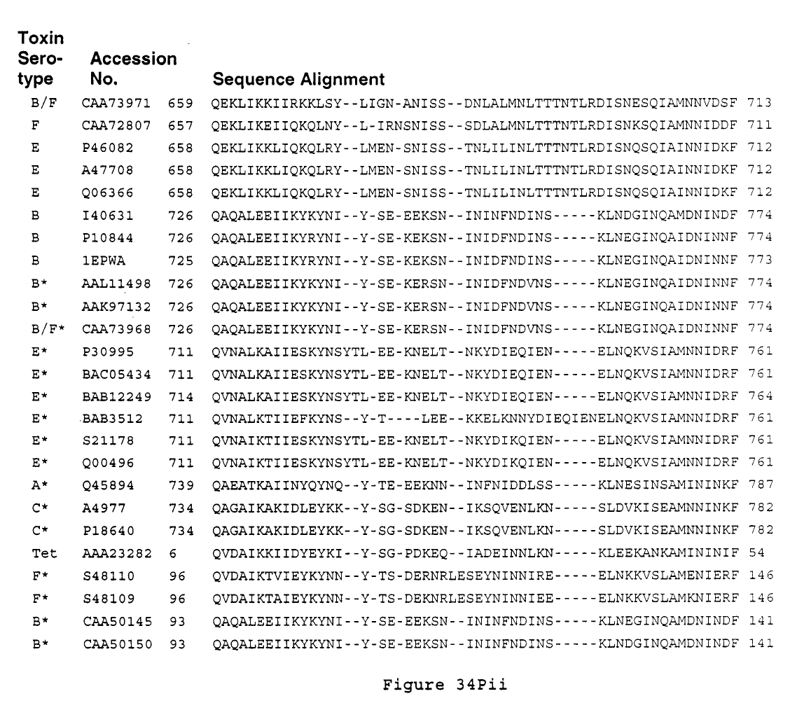

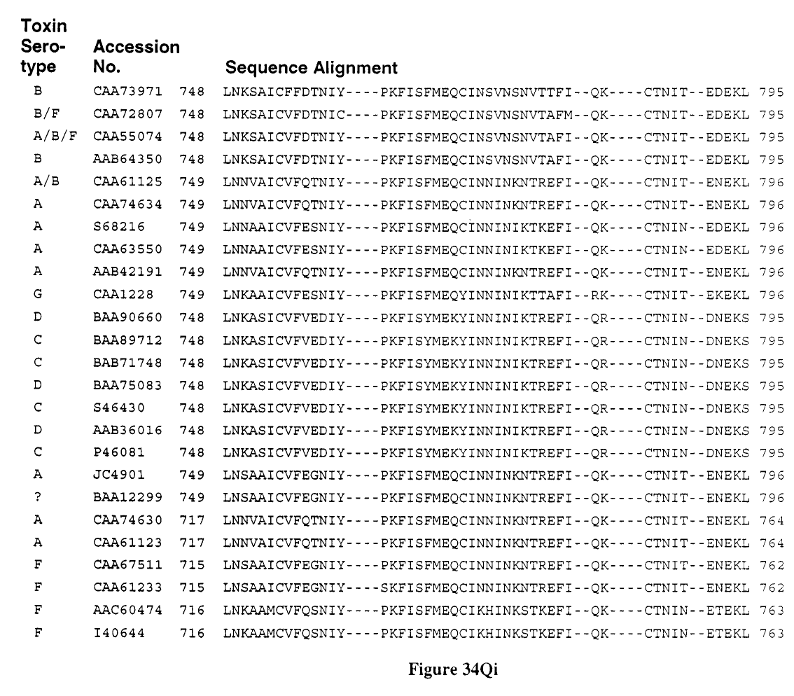

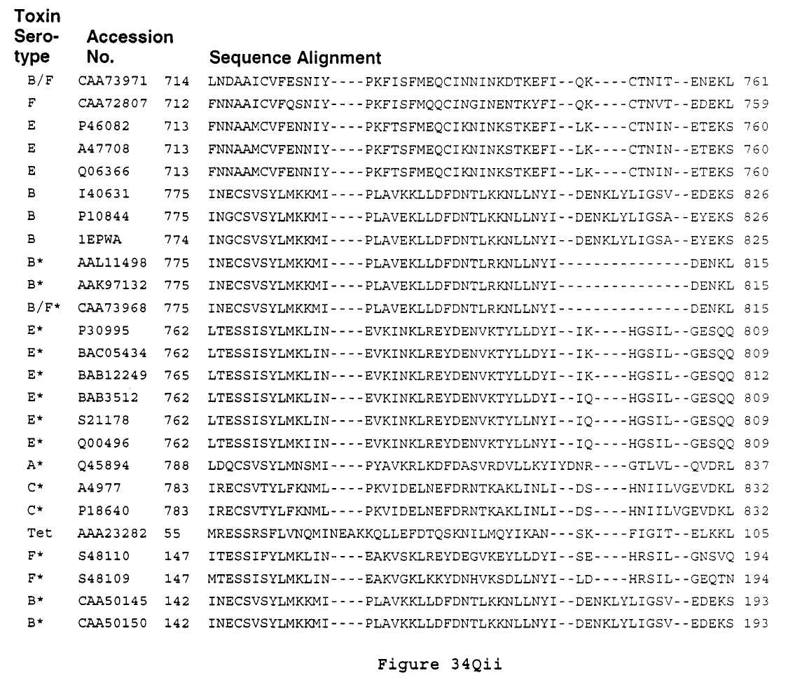

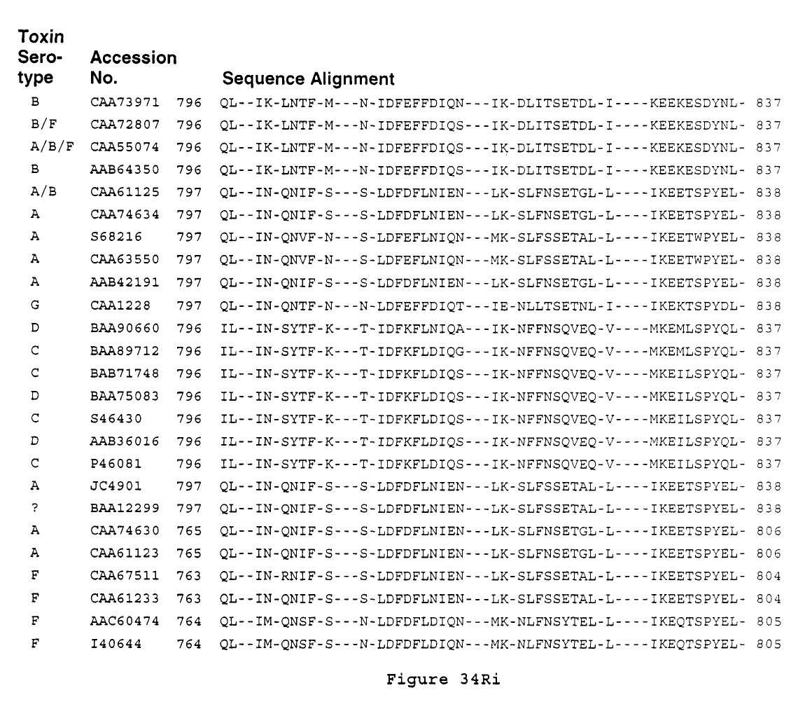

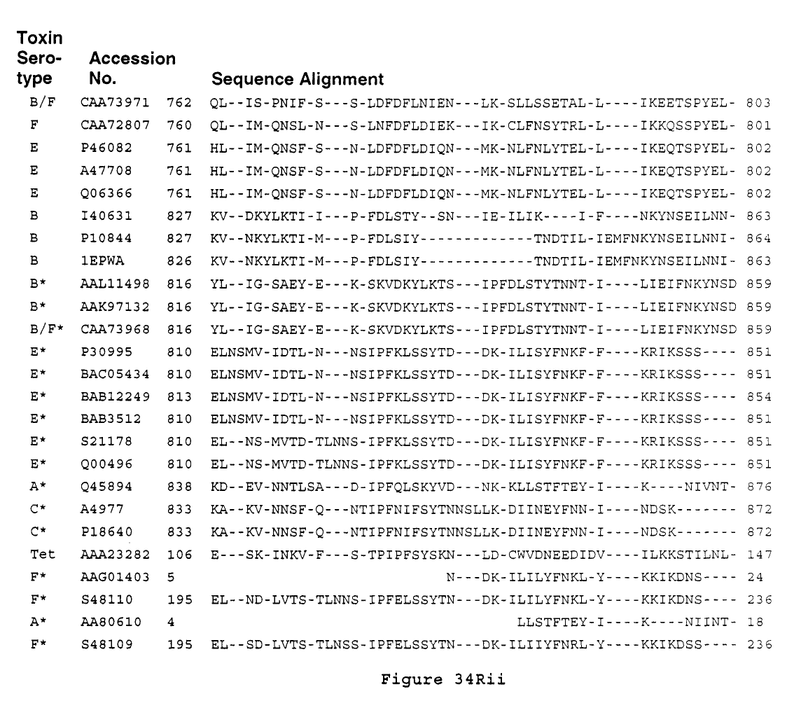

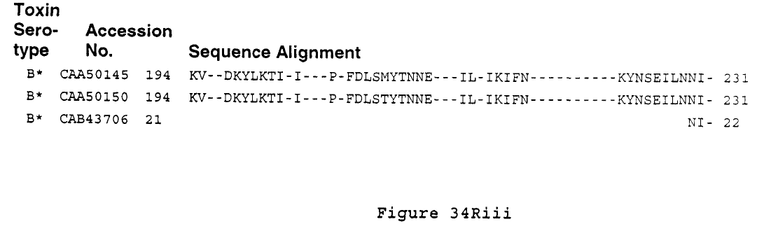

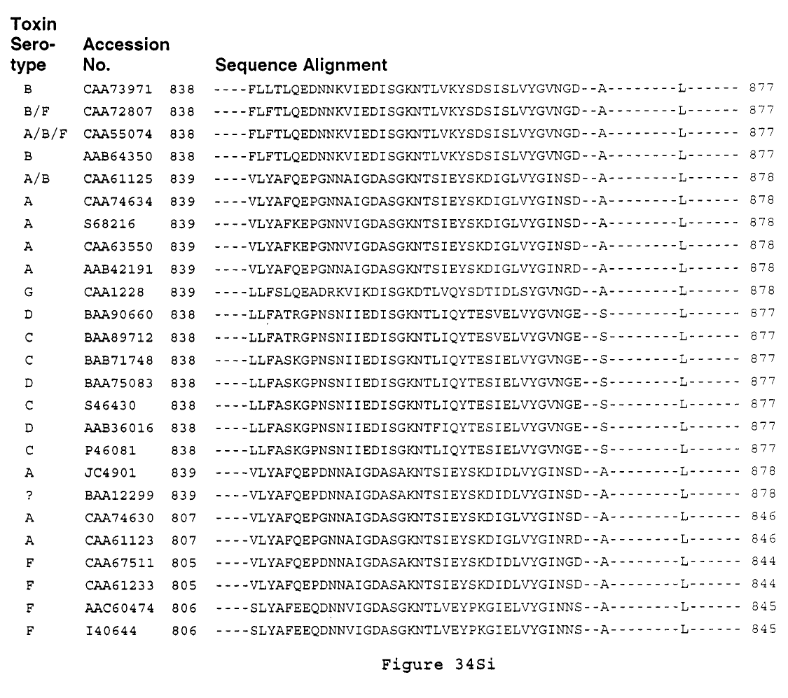

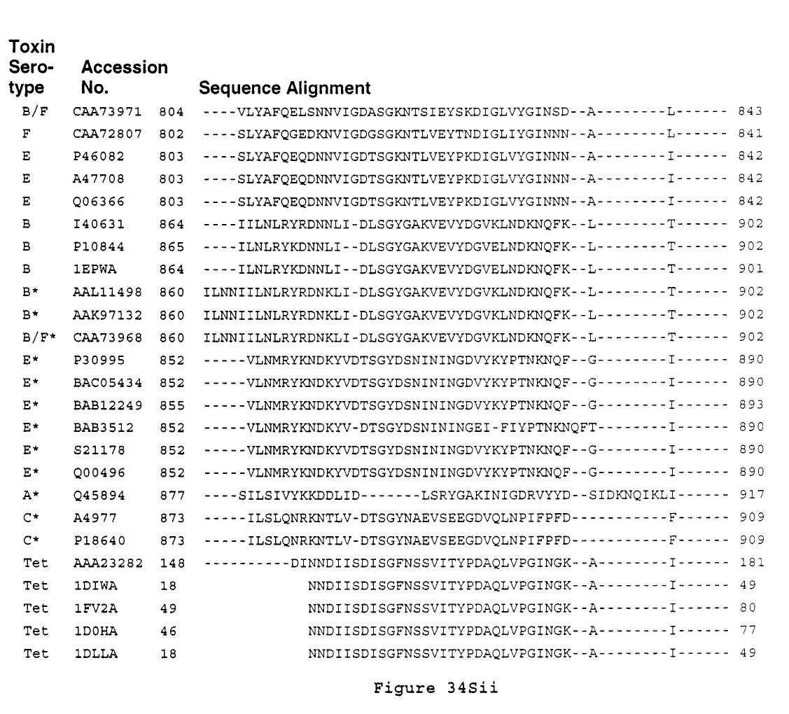

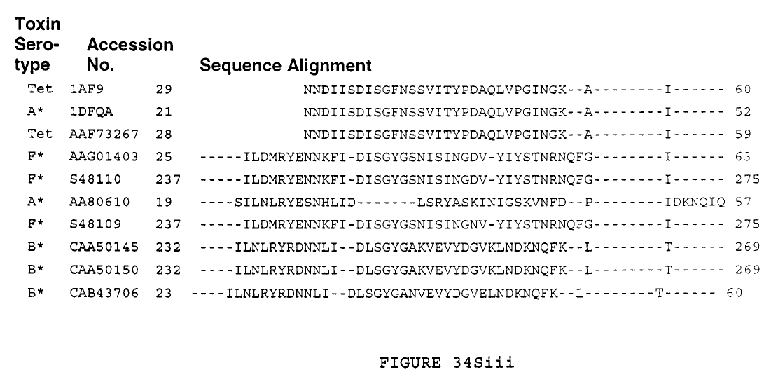

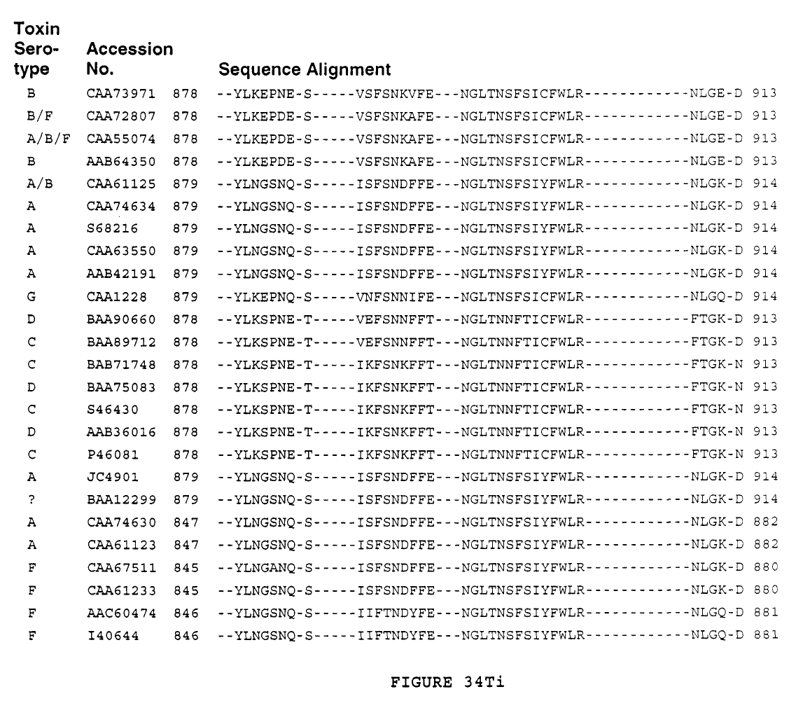

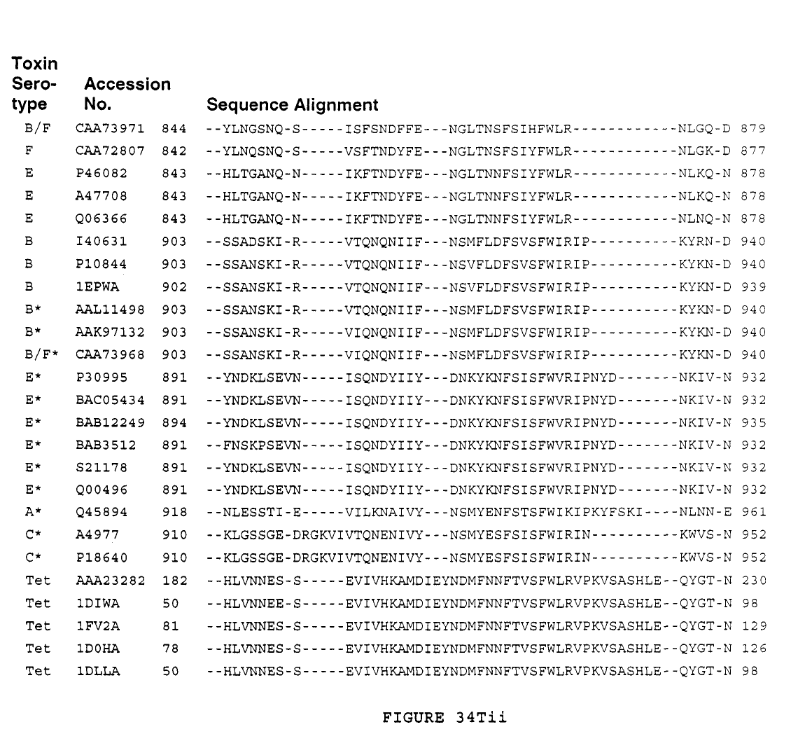

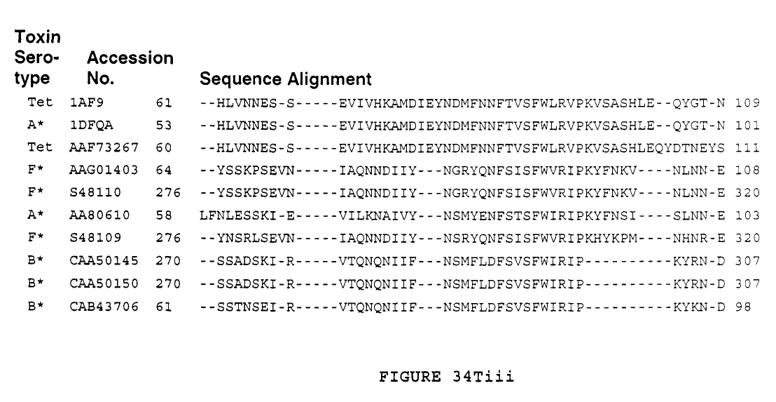

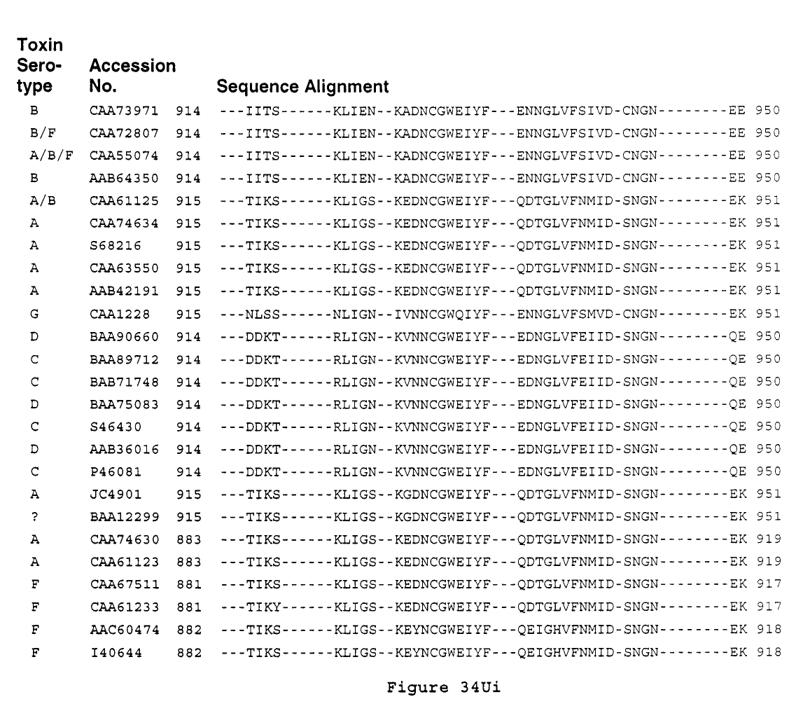

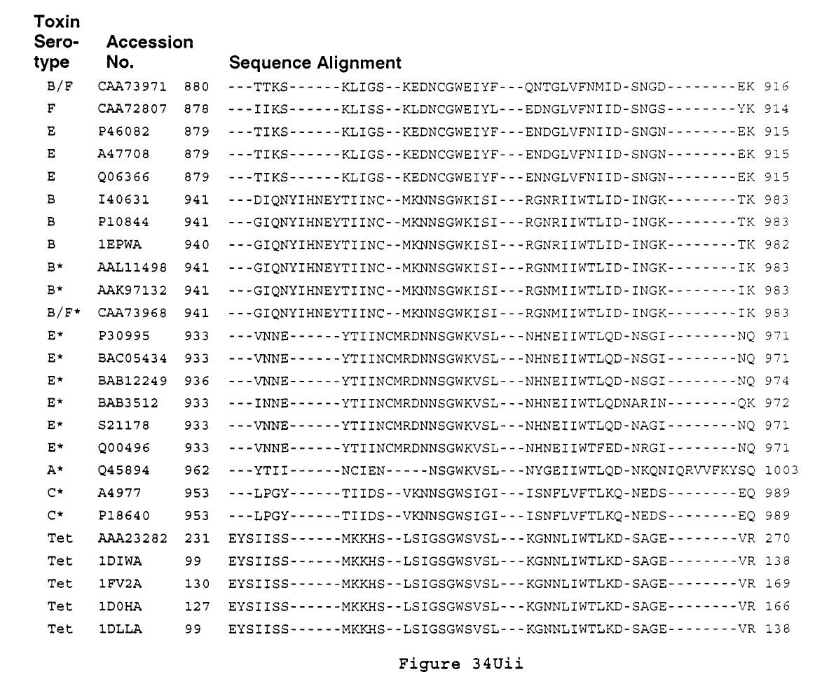

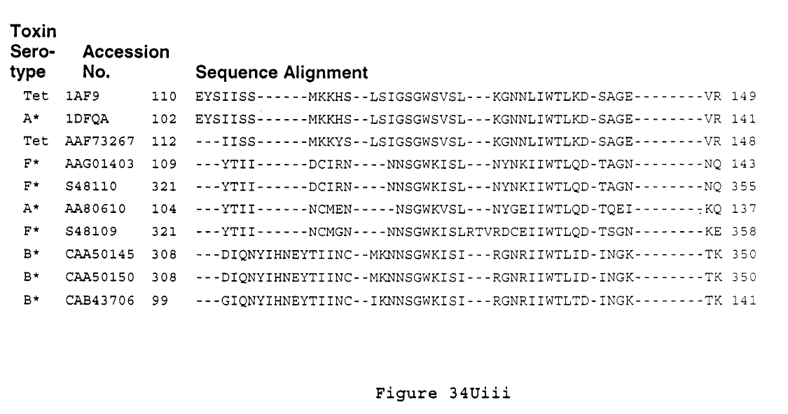

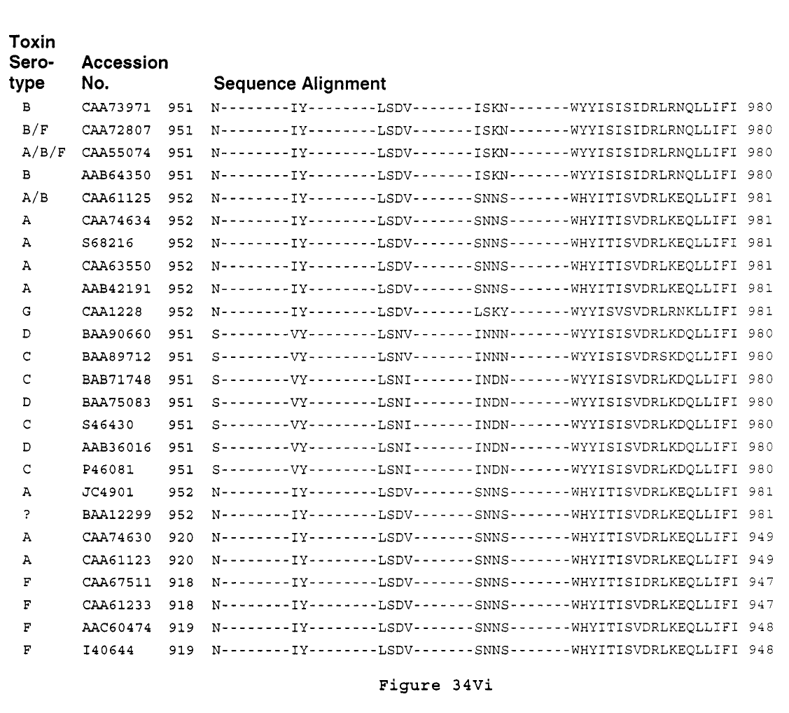

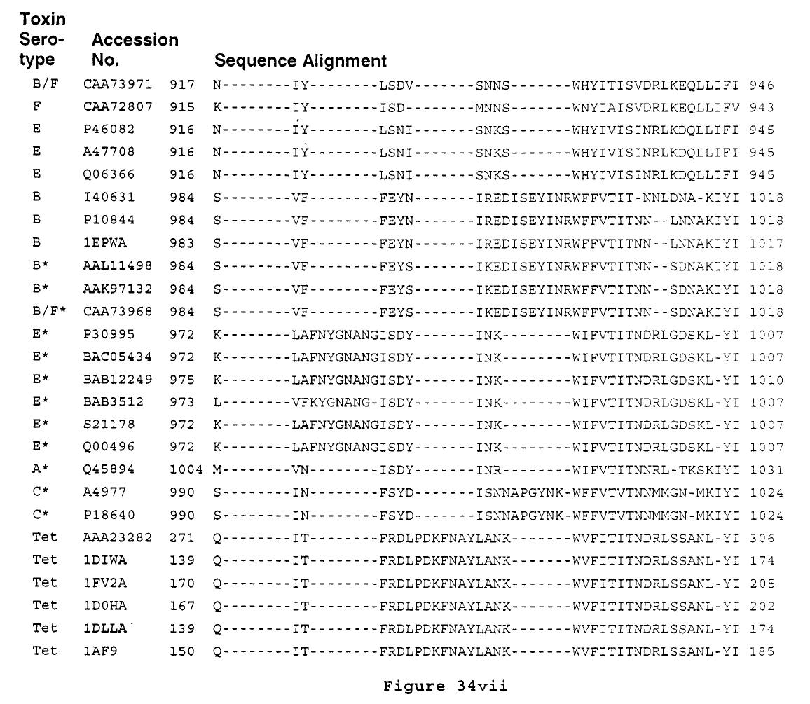

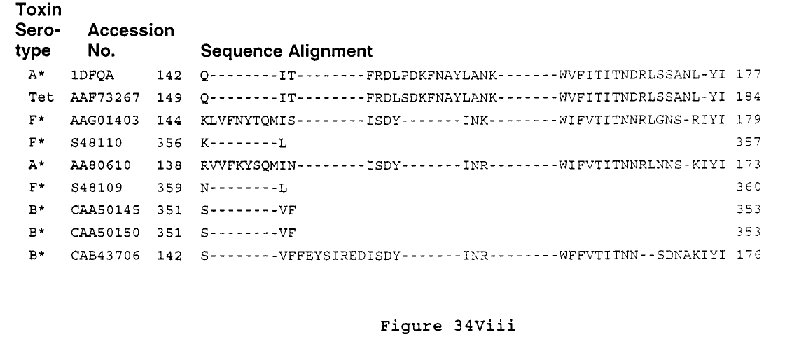

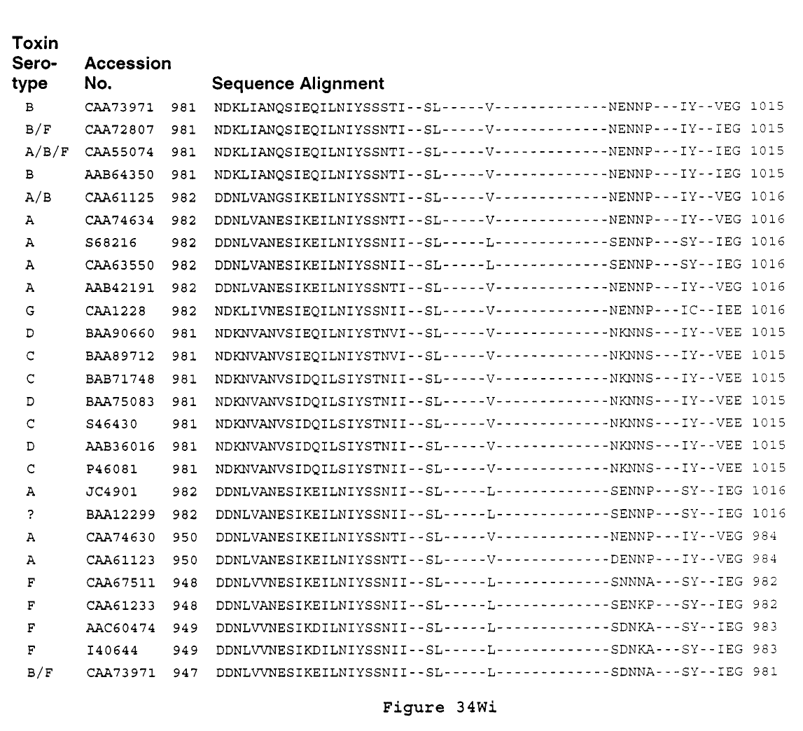

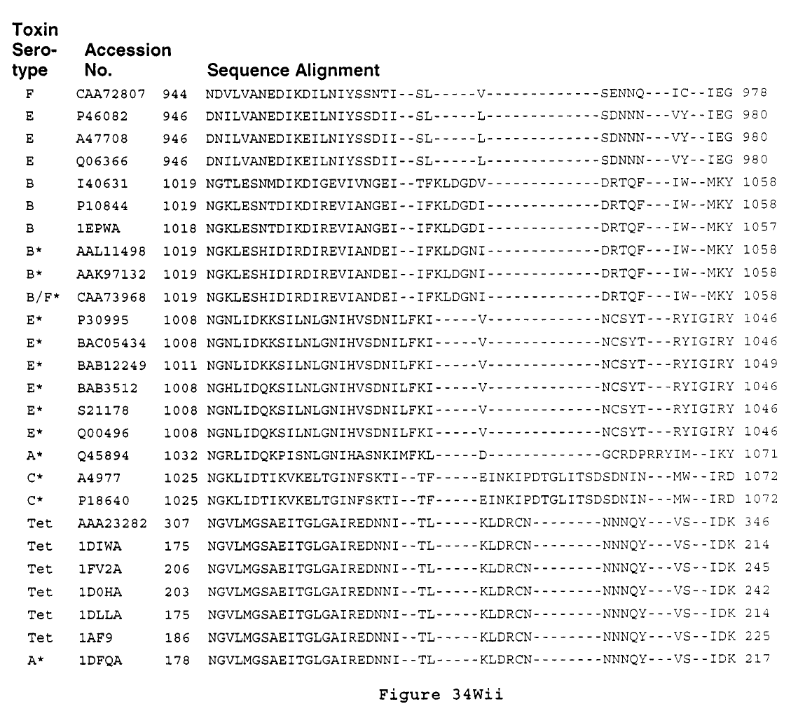

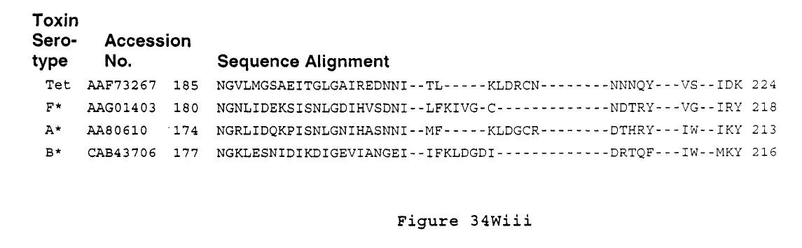

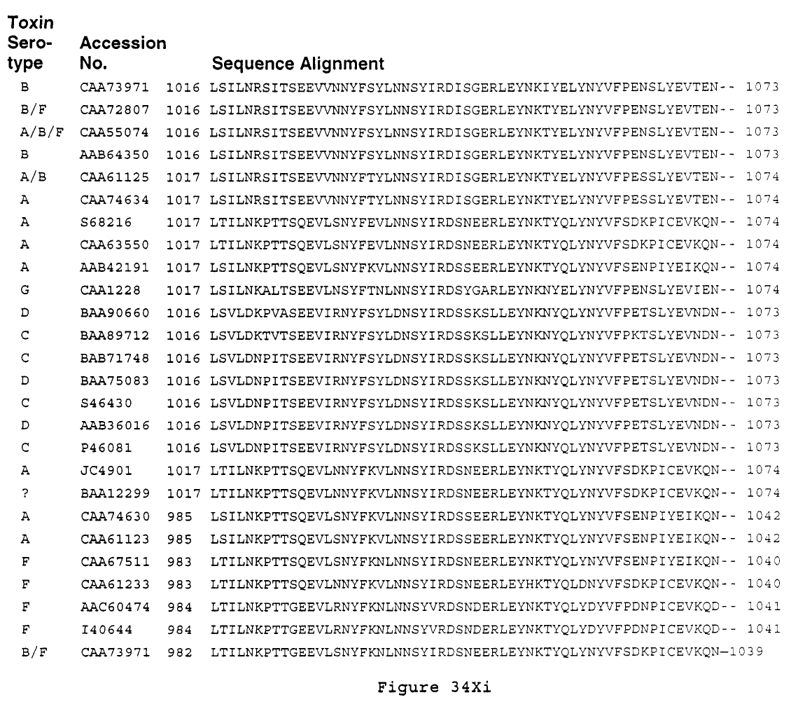

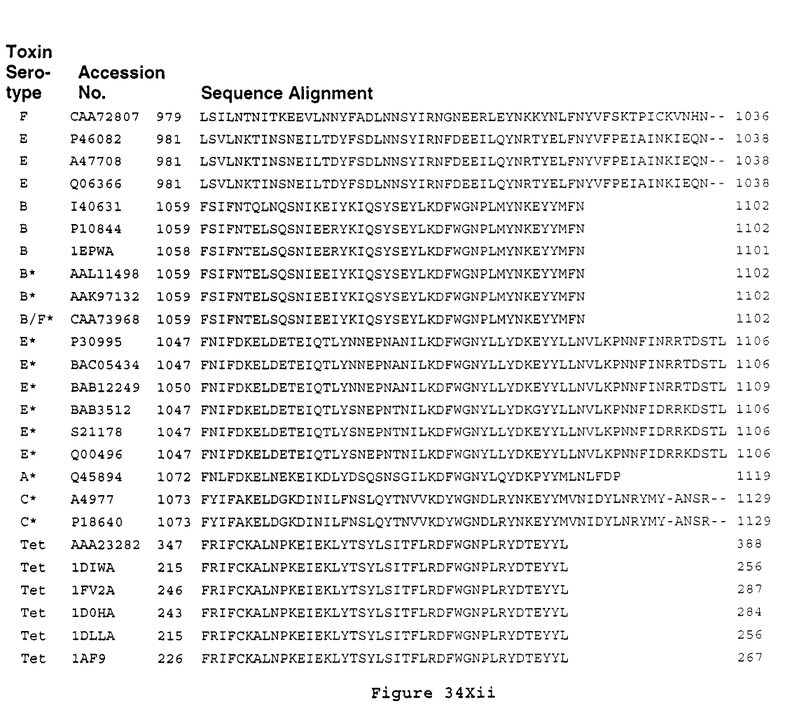

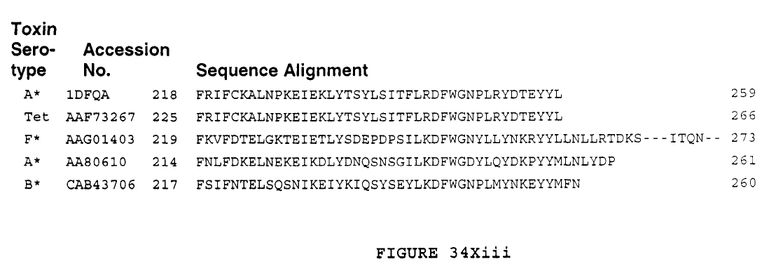

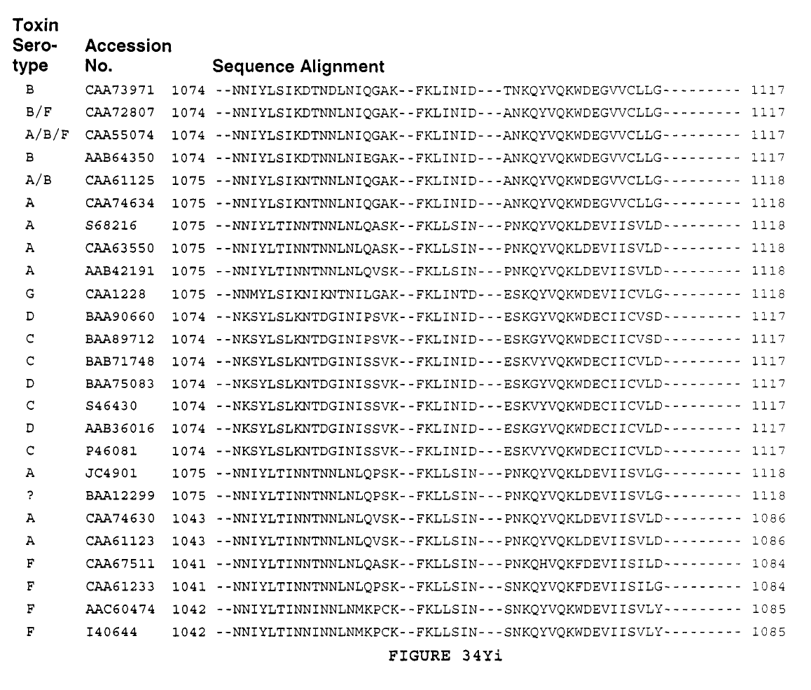

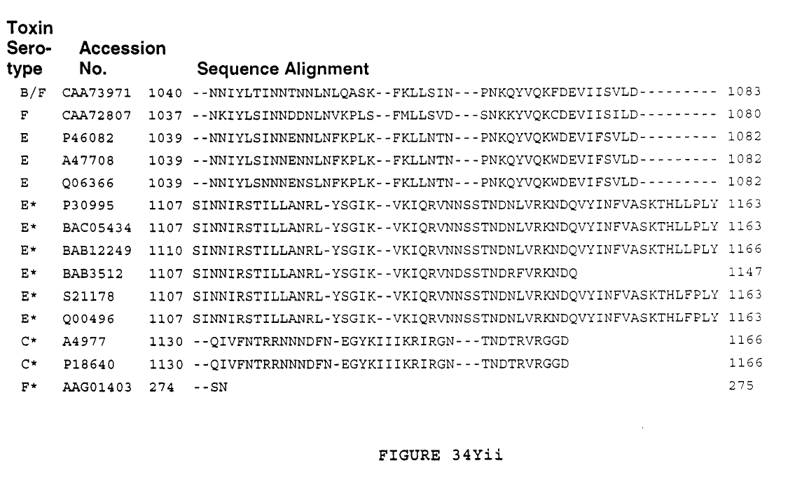

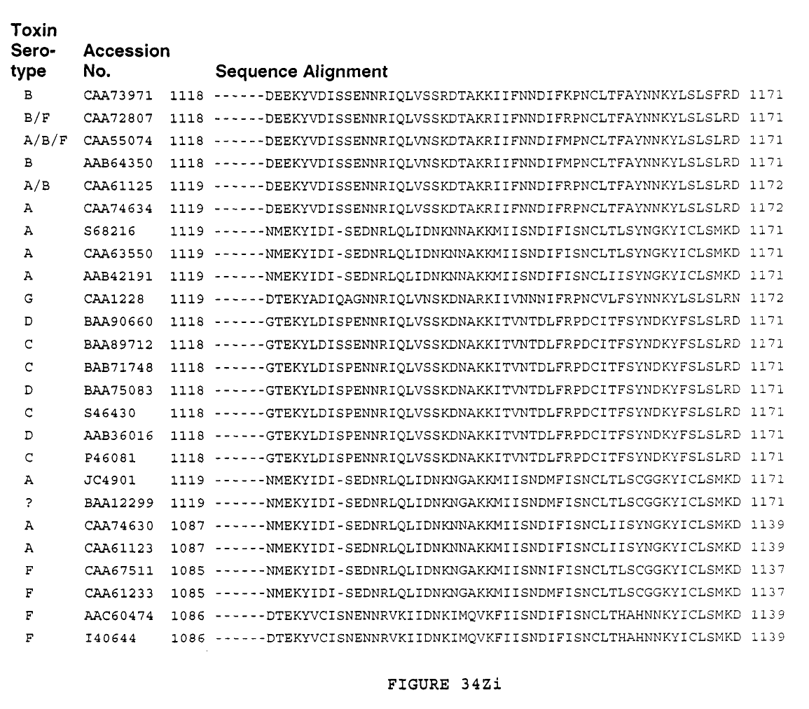

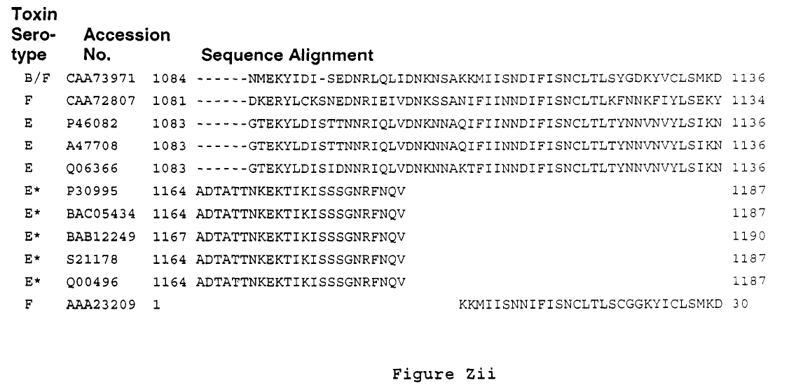

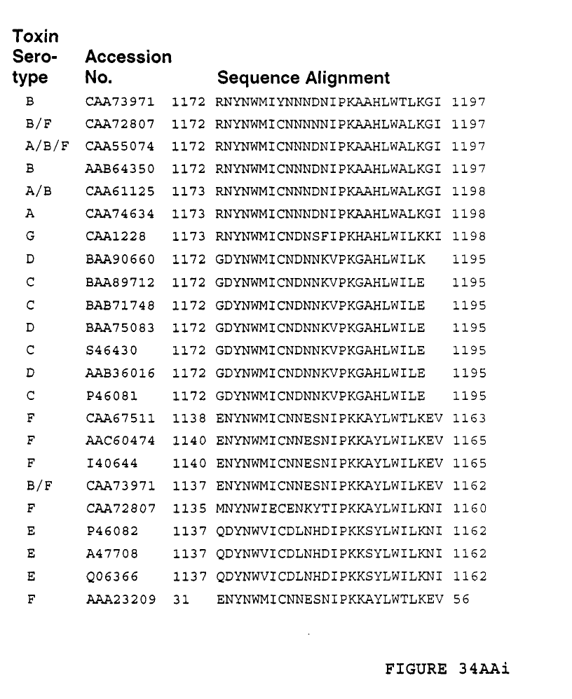

Alternatively, it may be preferred that the carboxyterminal HC fragment is a polypeptide that has an amino acid sequence that is at least 2% (by molecular mass) of the amino acid sequence of the full length HC of BoNT, with the fragment being obtained by excising/omitting the undesired portion of the full length HC beginning at the amino terminus (i.e., beginning at residue 468 in serotype A of FIG. 1). More preferably, the carboxyterminal HC fragment is a polypeptide that has the amino acid sequence of a portion that comprises at least about 5%, at least about 30%, at least about 50%, at least about 60%, at least about 70%, at least about 80%, and at least about 90% (by molecular mass) of the amino acid sequence of the full length HC of BoNT.

For example, the carboxyterminal HC fragments, serotype A, herein designated “88 kHC” (eighty-eight kilodalton carboxy fragment of BoNT HC), “66 kHC” (sixty-six kilodalton carboxy fragment of BoNT HC), and “50 kHC” (fifty kilodalton carboxy fragment of BoNT HC) are carboxyterminal HC fragments that are polypeptides that have an amino acid sequence of at least about 80%, at least about 70%, and at least about 50% of a portion of the full length BoNT HC sequence, respectively.

The carboxyterminal HC fragment for use in the invention can be derived from the HC of any of the known BoNT serotypes (e.g., any of A, B, C, D, E, F, and G) or from any subsequently discovered BoNT serotype. Preferably, the carboxyterminal HC fragment will be derived from a serotype that is known to be pathogenic in animals of the same species as the animal to which the composition is to be administered (e.g., serotypes A, B, and E for humans). However, it is not necessary that the selected fragment be obtained from a serotype that ordinarily exhibits pathogenicity in a given animal in order to retain transcytotic capacity in that animal.

The suitability for use in the invention of a carboxyterminal fragment HC obtained from a specific serotype or of a fragment having a specific amino acid sequence can be empirically assessed using routine scientific protocols, for example using a model system in which the fragment is contacted with cells of the same type and species to which administration is contemplated and transepithelial transport is observed and evaluated.

It is recognized that not all fragments will exhibit maximally efficient transcytosis in every epithelial cell type in every species. For example, the experiments described in the examples of this disclosure demonstrate that BoNTs, HCs, and/or fragments of serotypes A and B, do not appear to cross canine kidney epithelial membranes of a certain cell type (MDCK) with high efficiency. The fact that there may be specific embodiments of the invention that are not characterized by highly significant transcytotic abilities across certain epithelial cell types in certain species does not detract from the efficacy of the compositions and methods described herein.

It is preferred that the carboxyterminal HC fragment used in the methods and compositions of this invention possess at least the portion of the HC that is responsible for the toxin's ability to bind cell membranes. For example, the fragment may comprise the β-trefoil domain and/or the associated lectin binding domain. Alternatively, the carboxyterminal HC fragment may comprise a peptidomimetic of the HC that possesses the same binding properties as the native HC, as empirically determined by routine binding assays.

The identity or nature of the entity that is linked to the carboxyterminal HC fragment may be any entity that one desires to translocate across an epithelial surface. The entity or entities that is/are linked to the carboxyterminal HC fragment may be of virtually any size, as long as the size of the entity is not greater than the luminal capacity of vesicles of the cells to which the composition is to be administered. It is preferred that the selected entity or entities that is/are linked to the BoNT HC fragment has a molecular mass of about a few hundred daltons (about 100 daltons to about 200 daltons) to about a few tens of thousands of daltons (about 10,000 daltons to about 40,000 daltons). More preferred are entities that have molecular mass that is no greater than about 1000 daltons, and most preferred are entities of molecular mass of about 300 daltons to about 550 daltons. Alternatively, the molecular mass of the entity may be hundreds of thousands, millions, or tens of millions of daltons or more. Thus, transepithelial transport of very large supra-molecular complexes (e.g., a liposome or a virus vector) is contemplated.

Suitable entities that can be linked to the carboxyterminal HC fragments of the invention may include particles of organic or inorganic materials (for example, ceramic particles), small organic or inorganic chemical compounds (tetrafluroethylene polymers, chitosans), polypeptides (including, for example, single- and multi-subunit proteins such as enzymes, antibodies, and polypeptide epitopes of a pathogen), nucleic acids, and nucleic acid vectors (e.g., virus vectors containing an expressible nucleic acid).

In an embodiment, the entity comprises an immunogenic epitope of a pathogen of the animal. The composition having the immunogenic epitope linked to a carboxyterminal HC fragment facilitates delivery of the epitope to the bloodstream of the animal, thereby inducing generation of an immune response against the epitope. The immunogenic epitope may be protein or non-protein. The immune response provoked can thereafter inhibit or prevent pathology caused by the pathogen in the animal. Non-protein antigens from which suitable epitopes may be obtained include carbohydrates and nucleic acids.

Thus, in an embodiment, the compositions described herein are useful as vaccines for inducing protective immunity in vertebrates, such as mammals, reptiles or fish, when the entity to which the carboxyterminal HC fragment is linked is immunogenic. The compositions and methods described herein can be used for vaccination against substantially any human or other vertebrate pathogen (viral, bacterial, prion), including pathogens that may be weaponized and used as agents of biological warfare, as are known or to be developed in the art.

For example, the pathogen against which the vaccine compositions of the invention may be formulated can be Plasmodium falciparum (the causative agent of malaria), Bordetella pertussis (the causative agent of whooping cough), measles viruses, mumps viruses, rubella viruses, influenza viruses, hepatitis viruses, Pneumococcal viruses, varicella viruses, rabies viruses, and the human immunodeficiency virus. Additionally, the immunogenic epitope for use as an entity can be selected to provoke an immune response against, for example, the pathogens Bacillus anthracis (causative agent of anthrax), Pseudomonas pseudomallei, Clostridium botulinum toxin (causative agent of botulism), Yersinia pestis (causative agent of the plague), Vibriocholera, Variola major (causative agent of smallpox), Francisella tularensis (causative agent of tularemia), virus(es) that are the causative agents of viral hemorrhagic fevers (e.g., Crimean-Cong hemorragic fever virus), Corynebacterium diptheriae, Coxiella burnetti (causative agent of Q fever), organisms of the genus Brucella (e.g., Brucella abortus, Brucella suis, Brucella melitensis, Brucella canis) (causative agent(s) of brucellosis), saxitoxin, Burkholderia mallei (causative agent of glanders), the ricin toxin of Ricinus communis, the epsilon toxin of Clostridium perfringens, Clostridiom tetani, Staphylococcus enterotoxin B, Nipah virus, Hantavirus, Rift Valley fever virus, virus(es) that are the causative agents of tick-borne encephalitis, Staphylococcal enterotoxin B, trichothecene mycotoxins, the causative agent of Yellow fever, the causative agents of multi-drug resistant tuberculosis, and the coronavirus that is the causative agent of Severe Acute Respiratory Syndrome (SARS). The immunogenic epitope that is the entity may also be an epitope that provokes immunity against insect or reptile venom and against various parasites.

The entity or entities may comprise a molecule that is able to bind specifically with another molecule in the bloodstream of the animal to which the composition is to be administered. Such entities include, but are not limited to, antibody substances such as tetra-subunit immunoglobulins and single-chain antibodies and individual members or fragments of receptor-ligand binding pairs (e.g., tumor necrosis factor alpha and its cell-surface receptor). An “antibody substance” means an immunoglobulin molecule or an immunologically active portion of an immunoglobulin molecule, i.e., a molecule that contains an antigen binding site which specifically binds an antigen. A molecule that specifically binds with an antigen is a molecule that binds the antigen but does not substantially bind other molecules. Examples of immunologically active portions of immunoglobulin molecules include the F(ab) and the F(ab′)₂ fragments which can be generated by treating the antibody with an enzyme such as papain or pepsin, respectively. The term also includes polyclonal and monoclonal antibodies. The term “monoclonal antibody” or “monoclonal antibody composition” refers to a population of antibody molecules that contain only one species of an antigen binding site capable of immunoreacting with a particular epitope.

In yet another important embodiment, the entity is an agent that exhibits a catalytic or biological activity in vivo. Examples of such agents include cytotoxins, such as ricin; enzymes, such as proteases (e.g., tissue-type plasminogen activators or urokinase-type plasminogen activators); and enzyme inhibitors. The entity can also be a detectable label, such as a contrast agent, a radio-labeled antibody substance, or a radioisotope, such as ³H, ³⁵S, ¹²³I, and/or ¹³¹I.

It may be desirable that the linked entity is a larger entity that encompasses or incorporates numerous smaller therapeutic or immunogenic agents. For example, entities which may be used to transport numerous smaller therapeutic, diagnostic or immunogenic agents include liposomes, resealed RBCs, micelles, microspheres, and microparticles.

In the composition of the invention, the selected entity (or entities) is linked to the selected carboxyterminal HC fragment. A single fragment can be linked to one or more entities that are the same or are different. Alternatively, a single entity can be linked with multiple fragments (each of which may be the same or different).

The entity may be linked to the carboxyterminal HC fragment at any location, as long as the transcytotic capability of the fragment is not significantly impaired. For example, the entity may be linked to or near carboxy terminus of the fragment. It is preferred that the entity is linked at or near the amino terminus end of the fragment.

The nature of the linker may vary. The precise chemistry, linker, or method used to link the entity and the BoNT HC fragment is not critical. The linkage must merely be sufficiently strong or resilient that the fragment does not dissociate from the entity upon vesicular encapsulation of the fragment by the epithelial cell. The entity and the fragment may be linked by a covalent bond, such as, for example, a peptide bond. However, strong non-covalent linkage can also be used.

The linker may also be an intervening molecule, which may or may not have a chemical, therapeutic, or diagnostic function as well as its linking function. Examples of intervening molecules that can be used as linkers include biotin, avidin, or an antibody substance. Linkers of this type may be interposed between the entity and the fragment.

In the case where the selected entity or entities is a peptide or polypeptide, the entity can be linked to the selected fragment by peptide bond(s), thereby forming a unitary polypeptide comprising the entity and the fragment. For example, a unitary polypeptide can be prepared that comprises both the carboxterminal HC fragment and the selected entity. Such polypeptides can be prepared in any manner known or to be developed in the art, such as by expression of a fusion polypeptide from a recombinant expression vector, or by chemical synthesis, such as, for example, the solid-phase method.

The entity may also be linked by incorporation of the fragment into the entity itself. For example, if the entity is a liposome, the fragment may include a specific domain that permits a portion of the fragment to penetrate and be maintained within the structure of the liposome, while the transcytotic portion of the fragment remains unimpaired.

Depending on the desired use/route of administration intended for the final composition, methods can also be used to link the entity and the fragment in a chemically or biologically unstable or reversible manner. For example, the linkage may be enzyme cleavable by an enzyme co-administered to the patient or that is known to be present in the anatomical area to which the composition is delivered. Alternatively, one or more disulfide bonds may be used.

The entity and the fragment can be made separately and thereafter linked, or they can be made simultaneously.

In an embodiment, the composition of the invention may be a vaccine against the Clostridium botulinum toxin, in which the entity and the carboxyterminal HC fragment exist as an integral polypeptide molecule, and the linker is therefore a peptide bond. In this embodiment of the invention, the carboxyterminal HC fragment/entity integral polypeptide molecule comprises at least that portion of the sequence of the full length BoNT that encodes an immunogenic epitope that provokes an immune response in the animal for which the vaccine is intended.

Regardless of the epitope or the specific type of entity utilized, the immune response elicited may be a systemic immune response or a mucosal immune response. Depending on the circumstances in which the compositions and methods of the invention are to be applied, it may be desirable to elicit a mucosal immune response, rather than a systemic response, especially when the antigen against which immunity can be produced can be utilized in both a beneficial and a detrimental/toxic manner.

As an example, it is known that botulinum toxin is a potent toxin that is used as an agent of warfare or bioterrorism. Thus, immunization using the compositions and methods of the invention against botulinum toxin may be desirable. However, botulinum toxin is commonly used as a therapeutic agent to treat disorders that are characterized by an excessive and involuntary release of acetylcholine. Thus, an individual having a systemic immunity to botulinum toxin would be subsequently substantially foreclosed from receiving the benefits of botulinum toxin therapy.

Compositions of the invention that are vaccines that evoke substantially only mucosal immunity, thereby avoiding this problem, may be prepared by use of a composition that contains the vaccine of the invention and an adjuvant that selectively triggers substantially only mucosal immunity. Such adjuvants include, for example, cholera toxin B subunit or unmethylated oligonucleotides. The adjuvants can be associated with or linked to the fragment-linked antigen or the fragment itself, or the adjuvant(s) can be co-administered to the animal.

In another embodiment, the vaccine of the invention can be prepared so as to elicit substantially only mucosal immunity in the animal to which it is administered by including signaling molecules that promote mucosal immune response and/or inhibit systemic immune response in the composition of the invention. Such signaling molecules may include interleukins or transforming growth factors. The signaling molecules may be linked or otherwise associated with the fragment-linked antigen, the fragment itself, or may be co-administered to the animal with the compositions of the invention.

By use of the compositions and methods described herein, transepithelial transport of the composition of the invention can be accomplished, in most cases, unidirectionally—i.e., from the apical surface of the epithelium to the basolateral surface, or, alternatively from the basolateral surface to the apical surface. The selected epithelium may be any known, although the efficiency of transcytosis may vary depending on the species of vertebrate, the specific epithelium selected, and/or other chemical or physiological factors. For example, it has been demonstrated that, in certain canine kidney epithelial cell cultures, transport using carboxyterminal fragments of HC, serotypes A and B, is less efficient; thus, kidney epithelium is not preferred.

The epithelium to be crossed by the entity-linked carboxyterminal HC fragment is preferably non-keratinized, or has been rendered non-keratinized. Most epithelia other than skin are normally non-keratinized. However, de-keratinization or partial solubilization of skin tissue can enable transdermal use of the compositions and methods described herein. Examples of generally suitable epithelia include gastrointestinal (e.g., oral, esophageal, gastric, ileal, duodenal, jejunal, colon, and anal), nasal, pulmonary, vaginal, and ocular epithelia. Epithelia accessed by peritoneal administration of the compositions described herein can also be suitable.

A wide variety of animals are susceptible to infection or colonization by Clostridium botulinum. It is preferred that the compositions and methods of the invention are applied to these animals. Accordingly, the compositions described herein are preferably for use in substantially all vertebrates, and to induce physiological responses in non-vertebrate animals, such as insects.

One species of animals for which the compositions and methods described herein are intended is humans. For humans, use of carboxyterminal HC fragments derived from full length HCs of the A, B, and E serotypes are preferred. Additionally, many fragments from all the BoNT serotypes will be useful in common animals, such as house pets and farm animals. Thus, veterinary uses analogous to the pharmaceutical uses described herein are contemplated.

As will be recognized by a person of skill, the ability of a fragment to transcytose across an epithelium of a specific animal will vary depending on various factors, including the primary sequence of the fragment, the type/nature of the entity linked thereto, the serotype of BoNT from which the fragment was derived, etc. For example, fragments derived from serotype C may not comprise a most efficient transepithelial delivery in humans, but can be used to facilitate relatively effective and efficient delivery in non-human animals.

This differential capability among species facilitates use of the compositions of the invention for pesticidal and insecticidal purposes (i.e., by linking a fragment that does not transcytose across human epithelia with an entity that is a toxic agent). Suitable pesticidal or insecticidal agents may be those that exhibit greater toxicity toward pests or vermin than they do toward humans, and can be used to make pesticidal or insecticidal agents safer than many of those presently available. Such agents can be particularly useful in environments in which unavoidable exposure to humans is anticipated (e.g., in environments including children or food preparation facilities).

The invention also includes a foodstuff, wherein the genome of an ingredient of the foodstuff has been engineered to include a polynucleotide expressibly encoding a chimeric protein comprising an immunogenic or a therapeutic polypeptide linked to a carboxyterminal HC fragment. If desired, the chimeric protein may also be engineered to include one or more of the auxiliary proteins (HA(s) or NTNHs; SEQ ID NOs: 20 to 168, and 170 to 188), or other proteins or polypeptides.

Examples of ingredients which may be genetically modified accordingly include banana, potatoes, spinach, soybeans, and tomatoes. The ingredient can be administered individually to a human (e.g., by ingestion of an uncooked recombinant banana, tomato, or potato), or the ingredient can be incorporated into a prepared food comprising the ingredient (e.g., a fruit salad comprising pieces of recombinant banana).

The invention encompasses the preparation and use of medicaments and pharmaceutical or veterinary compositions comprising a carboxyterminal HC fragment having an entity linked thereto as an active ingredient. Such a pharmaceutical composition may consist of the linked active ingredient alone, in a form suitable for administration to a subject, or the pharmaceutical composition may comprise the linked active ingredient and one or more pharmaceutically acceptable vehicles, one or more additional ingredients, or some combination of these. Administration of one of these pharmaceutical compositions to a subject is useful for treating, ameliorating, relieving, inducing an immune response against, preventing, inhibiting, or reducing any of a variety of disorders in the subject, as described elsewhere in the present disclosure. The active ingredient may be present in the pharmaceutical composition in the form of a physiologically acceptable ester or salt, such as in combination with a physiologically acceptable cation or anion, as is well known in the art.

As used herein, the term “pharmaceutically acceptable vehicle” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition and which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a vehicle or one or more other accessory ingredients, and then, if necessary or desirable, shaping or packaging the product into a desired single- or multi-dose unit.

Although the descriptions of pharmaceutical compositions provided are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals, including commercially relevant mammals; such as cattle, pigs, horses, sheep, cats, and dogs; birds, including commercially relevant birds such as chickens, ducks, quail, geese, and turkeys; fish including farm-raised fish and aquarium fish; and crustaceans such as farm-raised shellfish and mollusks.

Pharmaceutical compositions that are useful in the methods of the invention may be prepared, packaged, or sold in formulations suitable for gastrointestinal, oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable vehicle, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient. A unit dose of a pharmaceutical composition of the invention will generally comprise from about 1 microgram to about 1 gram of the active ingredient, and preferably comprises from about 100 micrograms to about 100 milligrams of the active ingredient.

In addition to the active ingredient, a pharmaceutical composition of the invention may further comprise one or more additional pharmaceutical agents. Particularly contemplated additional agents include ingredients which can shield the BoNT HC fragment-entity complex from the effects of the acidic pH environment of portions of the gastrointestinal tract. Substantially all formulations and devices for effecting enteric delivery known or to be developed can be used. Further, as discussed above, the pharmaceutical composition may contain the one or more of the auxiliary proteins (e.g., SEQ ID NOs: SEQ ID NOs: 20 to 168, and 170 to 188) associated in nature with the BoNT toxin (HA(s) and NTNHs), especially if the composition is to be administered orally or via any portion of the gastrointestinal tract. Although it is known in the art that varying proteins are associated with varying serotypes of BoNT, it is not necessary that this correspondence is maintained in the practice of the invention.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable vehicle, and at least sufficient liquid to moisten the mixture. Pharmaceutically acceptable excipients used in the manufacture of tablets include, but are not limited to, inert diluents, granulating and disintegrating agents, binding agents, and lubricating agents. Suitable dispersing agents include, but are not limited to, potato starch and sodium starch glycolate. Known surface active agents include, but are not limited to, sodium lauryl sulfate. Known diluents include, but are not limited to, calcium carbonate, sodium carbonate, lactose, microcrystalline cellulose, calcium phosphate, calcium hydrogen phosphate, and sodium phosphate. Suitable granulating and disintegrating agents include, but are not limited to, corn starch and alginic acid. Binding agents include, but are not limited to, gelatin, acacia, pre-gelatinized maize starch, polyvinylpyrrolidone, and hydroxypropyl methylcellulose. Lubricating agents include, but are not limited to, magnesium stearate, stearic acid, silica, and talc.

Tablets may be non-coated or they may be coated using known or to be developed methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Oral compositions may be made, using known technology, which specifically release orally-administered agents in the small or large intestines of a human patient. For example, formulations for delivery to the gastrointestinal system, including the colon, include enteric coated systems, based, e.g., on methacrylate copolymers such as poly(methacrylic acid, methyl methacrylate), which are only soluble at pH 6 and above, so that the polymer only begins to dissolve on entry into the small intestine. The site where such polymer formulations disintegrate is dependent on the rate of intestinal transit and the amount of polymer present. For example, a relatively thick polymer coating is used for delivery to the proximal colon (Hardy et al., 1987 Aliment. Pharmacol. Therap. 1:273-280). Polymers capable of providing site-specific colonic delivery can also be used, wherein the polymer relies on the bacterial flora of the large bowel to provide enzymatic degradation of the polymer coat and hence release of the drug. For example, azopolymers (U.S. Pat. No. 4,663,308), glycosides (Friend et al., 1984, J. Med. Chem. 27:261-268) and a variety of naturally available and modified polysaccharides (PCT GB89/00581) may be used in such formulations.

Pulsed release technology such as that described in U.S. Pat. No. 4,777,049 may also be used to administer the active agent to a specific location within the gastrointestinal tract. Such systems permit drug delivery at a predetermined time and can be used to deliver the active agent, optionally together with other additives that my alter the local microenvironment to promote agent stability and uptake, directly to the colon, without relying on external conditions other than the presence of water to provide in vivo release.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Emulsifying agents include, but are not limited to, lecithin and acacia. Preservatives include, but are not limited to, methyl, ethyl, or n-propyl-para-hydroxybenzoates, ascorbic acid, and sorbic acid. Sweetening agents include, for example, glycerol, propylene glycol, sorbitol, sucrose, and saccharin. Thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils, such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid vehicle. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid vehicle. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intravenous, intra-arterial, intramuscular, or intrasternal injection and intravenous, intra-arterial, or kidney dialytic infusion techniques.

Formulations of a pharmaceutical composition suitable for parenteral administration comprise the fragment-linked active ingredient combined with a pharmaceutically acceptable vehicle, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules, in multi-dose containers containing a preservative, or in single-use devices for auto-injection or injection by a medical practitioner. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein. Topically administered formulations should be adapted for application to a non-keratinized epithelial tissue (e.g., the inside of the mouth, nose, or throat), and can be provided together with an applicator or dispenser for achieving such application.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid vehicle. Such drops may further comprise buffering agents, salts, or one or more other of the additional ingredients described herein. Other ophthalmalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., U.S.A., which is incorporated herein by reference.

It is understood that the ordinarily skilled physician or veterinarian will readily determine and prescribe an effective amount of the compound to treat, ameliorate, relieve, inhibit, prevent, reduce a disorder in the subject or to elicit an immune response. In so proceeding, the physician or veterinarian may, for example, prescribe a relatively low dose at first, subsequently increasing the dose until an appropriate response is obtained. It is further understood, however, that the specific dose level for any particular subject will depend upon a variety of factors including the activity of the specific compound employed, the age, body weight, general health, gender, and diet of the subject, the time of administration, the route of administration, the rate of excretion, any drug combination, and the severity of the disorder.

Another aspect of the invention relates to a kit comprising a pharmaceutical composition of the invention and an instructional material. As used herein, an “instructional material” includes a publication, a recording, a diagram, or any other medium of expression which is used to communicate the usefulness of the pharmaceutical composition of the invention for treating, ameliorating, relieving, inhibiting, preventing, or reducing a disorder in a subject or for administering such a composition via a route described herein. The instructional material may also, for example, describe an appropriate dose of the pharmaceutical composition of the invention. The instructional material of the kit of the invention may, for example, be affixed to a container which contains a pharmaceutical composition of the invention or be shipped together with a container which contains the pharmaceutical composition. Alternatively, the instructional material may be shipped separately from the container with the intention that the instructional material and the pharmaceutical composition be used cooperatively by the recipient.

The invention also includes a kit comprising a pharmaceutical composition of the invention and a delivery device for delivering the composition to a subject. By way of example, the delivery device may be a squeezable spray bottle, a metered-dose spray bottle, an aerosol spray device, an atomizer, a dry powder delivery device, a self-propelling solvent/powder-dispensing device, a syringe, a needle, a tampon, or a dosage measuring container. The kit may further comprise an instructional material as described herein.

The invention is now described with reference to the following Examples. These Examples are provided for the purpose of illustration only. The invention is not limited to these Examples, but rather encompasses all variations which are evident as a result of the teaching provided herein.

### EXAMPLES

The results of experiments detailed in the Examples are summarized in Table 1.

TABLE 1

| Example | | | Cell | Transport | Transport | Transport Rate |
|---|---|---|---|---|---|---|
| No. | Molecule^{A} | Serotype | Type^{B} | A-->B^{C} | B-->A^{C} | (fmol/hr/cm²)^{D} |
| 1 | BoNT | A | T-84 | + | | FIG. 3 |
| 2 | BoNT | A | MDCK | − | | FIG. 4 |
| 3 | ¹²⁵I-BoNT | A | T-84 | + | | 11.29 ± 0.30 |
| 4 | ¹²⁵I-BoNT | A | T-84 | | + | 8.98 ± 0.20 |
| 5 | ¹²⁵I-BoNT | A | Caco-2 | + | | 8.42 ± 0.49 |
| 6 | ¹²⁵I-BoNT | A | MDCK | + | | 0.05 ± 0.01 |
| 7 | ¹²⁵I-uBoNT | B | T-84 | + | | 9.01 ± 0.44 |
| 8 | ¹²⁵I-uBoNT | B | MDCK | − | | 0.05 ± 0.00 |
| 9 | ¹²⁵I-uBoNT | B | T-84 | | + | 4.48 ± 0.00 |
| 10 | ¹²⁵I-uBoNT | B | MDCK | | − | 0.05 ± 0.00 |
| 11 | ¹²⁵I-nBoNT | B | T-84 | + | | 6.57 ± 0.07 |
| 12 | ¹²⁵I-nBoNT | B | MDCK | − | | 0.05 ± 0.00 |
| 13 | ¹²⁵I-nBoNT | B | T-84 | | + | 5.54 ± 0.00 |
| 14 | ¹²⁵I-nBoNT | B | MDCK | | − | 0.05 ± 0.00 |
| 15 | HC | A | T-84 | + | | FIG. 5 |
| 16 | HC | A | MDCK | − | | FIG. 6 |
| 17 | ¹²⁵I-HC | A | T-84 | + | | 7.10 ± 0.00 |
| 18 | ¹²⁵I-HC | A | MDCK | − | | 0.06 ± 0.00 |
| 19 | 66 Khc | A | T-84 | + | | FIG. 7 |
| 20 | 66 kHC | A | MDCK | − | | FIG. 8 |
| 21 | 50 kHC | A | T-84 | + | | FIG. 9 |
| 22 | 50 kHC | A | MDCK | − | | |
| 23 | ¹²⁵I-50 kHC | A | T-84 | + | | 13.97 ± 0.00 |
| 24 | ¹²⁵I-50 kHC | A | MDCK | − | | 0.06 ± 0.00 |
| 25 | AF-BoNT | A | T-84 | + | | |
| 26 | AF-BoNT | A | MDCK | − | | |
| 27 | bt-50 kHC | A | T-84 | + | | |
| 28 | bt-50 kHC | A | MDCK | − | | |
| 29 | GFP-66 kHC | A | T-84 | + | | |
| 30 | GFP-66 kHC | A | MDCK | − | | |
| 31 | Stag-50 kHC | A | T-84 | + | | |
| 32 | Stag-50 kHC | A | MDCK | − | | |
| 33 | GST-88 kHC | A | T-84 | + | | |
| 34 | GST-88 kHC | A | MDCK | − | | |
| 35 | GST-66 kHC | A | T-84 | + | | |
| 36 | GST-66 kHC | A | MDCK | − | | |
| 37 | ¹²⁵I-BoNT | A | Calu-3 | + | | 0.422 ± 0.076 |
| 38 | ¹²⁵I-BoNT | A | Calu-3 | | + | 0.206 ± 0.037 |
| 39 | ¹²⁵I-HC | A | Calu-3 | + | | 0.198 ± 0.007 |
| 40 | ¹²⁵I-HC | A | Calu-3 | | + | 0.112 ± 0.033 |
| 41 | ¹²⁵I-BoNT | A | RAEC | + | | 0.376 ± 0.014 |
| 42 | ¹²⁵I-BoNT | A | RAEC | | + | 0.159 ± 0.027 |
| 43 | ¹²⁵I-HC | A | RAEC | + | | 0.140 ± 0.050 |
| 44 | ¹²⁵I-HC | A | RAEC | | + | 0.132 ± 0.026 |
| 45 | ¹²⁵I-BoNT | A | mRT | + | | |
| 46 | ¹²⁵I-HC | A | mRT | + | | |
| 47 | 6xHis-50 kHC | A | mRT | + | | |
| 48 | GST-50 kHC | A | mRT | + | | |
| 49 | GST-50 kHC | A | mRT | + | | |
| 50 | 6xHis-50 kHC | A | mRT | + | | ± |
| 51 | CTBS-50 kHC | A | mRT | + | | |
| 52 | full length HC | A | oral | + | | |
| | (100 kDa) | | | | | |
| 53 | HC and its | A & B | in vivo; | + | + | — |
| | fragments | | in vitro | | | |

^{A}Abbreviations are as follows:
BoNT = Clostridium botulinum nerurotoxin (holotoxin);
nBoNT = nicked BoNT, i.e., BoNT precursor polypeptide that has been cleaved into HC and LC, that are linked by a disulfide bond;
uBoNT = un-nicked BoNT i.e., BoNT precursor polypeptide (150 kDa) that remains uncleaved;
HC = heavy chain of Clostridium botulinum neurotoxin;
88 kHC = 88 kilodalton HC carboxyterminal fragment (see FIG. 2);
66 kHC = 66 kilodalton HC carboxyterminal fragment (see FIG. 2);
50 kHC = 50 kilodalton HC carboxyterminal fragment (see FIG. 2);
48 kHC = the 50 kHC fragment from which 2 kilodaltons of the carboxy terminus have been excised (see FIG. 2);
AF = ALEXA FLUOR ® 568 fluorescent dye;
bt = biotin;
GFP = green fluorescent protein;
GST = glutathione-S-transferase;
Stag = S-TAG ™ System (15aa S•Tag peptide with ribonuclease S-protein, available from Novagen, Inc., Madison, Wisconsin, U.S.A.);
6xhis = hexahistidine label; and
CTBS = cholera toxin B subunit;
^{B}Cell types are as follows:
T-84 = T-84 human gut epithelial cells;
MDCK = Madin-Darby canine kidney epithelial cells;
Caco-2 = Caco-2 human gut epithelial cells;
Calu-3 = Calu-3 human pulmonary epithelial cells;
RAEC = rat alveloar epithelial cells; and
mRT = murine respiratory tract cells, in vivo.
^{C}A = apical face of cells and B = basolateral face of cells.
^{D}Rates are reported as means ± standard error of the mean, in femtomoles per hour per square centimeter of epithelium surface.

^{A}Abbreviations are as follows:

BoNT=Clostridium botulinum neurotoxin (holotoxin);

nBoNT=nicked BoNT, i.e., BoNT precursor polypeptide that has been cleaved into HC and LC, that are linked by a disulfide bond;

uBoNT=un-nicked BoNT i.e., BoNT precursor polypeptide (150 kDa) that remains uncleaved;

HC=heavy chain of Clostridium botulinum neurotoxin;

88 kHC=88 kilodalton HC carboxyterminal fragment (see FIG. 2);

66 kHC=66 kilodalton HC carboxyterminal fragment (see FIG. 2);

50 kHC=50 kilodalton HC carboxyterminal fragment (see FIG. 2);

48 kHC=the 50 kHC fragment from which 2 kilodaltons of the carboxy terminus have been excised (see FIG. 2);

AF=ALEXA FLUOR® 568 fluorescent dye;

bt=biotin;

GFP=green fluorescent protein;

GST=glutathione-S-transferase;

Stag=S-TAG™ System (15aa S●Tag peptide with ribonuclease S-protein, available from Novagen, Inc., Madison, Wis., U.S.A.);

6×his=hexahistidine label; and

CTBS=cholera toxin B subunit.

^{B}Cell types are as follows:

T-84 =T-84 human gut epithelial cells;

MDCK=Madin-Darby canine kidney epithelial cells;

Caco-2=Caco-2 human gut epithelial cells;

Calu-3=Calu-3 human pulmonary epithelial cells;

RAEC=rat alveolar epithelial cells; and

mRT=murine respiratory tract cells, in vivo.

^{C}A=apical face of cells and B=basolateral face of cells.

^{D}Rates are reported as mean±standard error of the mean, in femtomoles per hour per square centimeter of epithelium surface.

The materials and methods used in the examples described herein, except as noted in the individual examples, are now described.

### Native and Recombinant Proteins Native Toxin and Native Chains.

Botulinum neurotoxin (BoNT), as well as the HC and light chain (LC) components, was isolated by standard techniques that have been well described in the literature (DasGupta and Sathyamoorthy, 1984; Simpson et al., 1988).

### Construction of the Plasmid Expressing the BoNT LC.

Standard techniques for DNA fragment isolation, repair of overhanging ends with the Klenow fragment of DNA polymerase I, and ligation with T4 DNA ligase were used. All cloning steps and expression were performed in E. coli M-15 (obtained from Qiagen, Chatsworth, Calif., U.S.A.) containing the pREP4 repressor plasmid. A DNA fragment coding for the BoNT LC (rL chain) was amplified from plasmid pCL8 using primers having the following sequences: forward, CCCAATAACA ATTAACAACT TTAAT (SEQ ID NO: 8); and reverse, TTTctgcagC TATTTRATTAT ATAATGATCT ACCATC (SEQ ID NO: 9), where the PstI restriction site is in lowercase characters. One cytosine was added to the 5′ end of the forward primer to provide for reconstruction of the BamHI restriction site, as well as to clone light-chain DNA in frame with the pQE-30 initiation of translation methionine. In the reverse primer, a PstI restriction site was introduced immediately downstream of the stop codon. The amplified product was purified, treated with T4 polymerase, cut with PstI, and inserted between the Klenow-filled-in BamHI and PstI restriction sites of the expression vector pQE-30 to yield plasmid pQE-LC1. The structure of pQE-LC1 was confirmed by DNA sequencing.

### Construction of Plasmid Expressing Truncation Mutants of HC (Carboxyterminal HC Fragment).

The structural gene (having the nucleotide sequence SEQ ID NO: 10, as listed in GENBANK™ accession no. X73423) encoding the eighty-eight kilodalton (88 K), sixty-six kilodalton (66 K), or fifty kilodalton (50 K) fragments of HC of BoNT serotype A (BoNT A) were generated by PCR. These HC fragments are herein designated “88 kHC,” “66 kHC,” and “50 kHC.”

DNA fragment (nucleotide residues 1609-3987 of SEQ ID NO: 10; SEQ ID NO: 11) encoding 88 kHC was amplified using the following oligonucleotide primers: forward primer (nucleotide residues 1609-1632 of SEQ ID NO: 10) CGCggtaccA CCTTTAATTT TGATAATGAA CCT (SEQ ID NO: 12), reverse primer (nucleotide residues 3987-3968 of SEQ ID NO: 10) AACCCctgca gTTACAGTGG CCTTTCTCCC C (SEQ ID NO: 13), where the KpnI restriction site in the forward primer sequence and the PstI restriction site in the reverse primer sequence are in lower case characters.

DNA fragment (nucleotide residues 2170-3987 of SEQ ID NO: 10; SEQ ID NO: 14) encoding 66 kHC was amplified using the following oligonucleotide primers: forward primer (nucleotide residues 2170-2193 of SEQ ID NO: 10) CGCggtaccG TTCAAACAAT AGATAATGCT TTA (SEQ ID NO: 15), reverse primer (nucleotide residues 3987-3968 of SEQ ID NO: 10) AACCCctgca gTTACAGTGG CCTTTCTCCC C (SEQ ID NO: 13), where the KpnI restriction site in the forward primer sequence and the PstI restriction site in the reverse primer sequence are in lower case characters.

DNA fragment (nucleotide residues 2689-3987 of SEQ ID NO: 10; SEQ ID NO: 16) encoding 50 kHC was amplified using the following oligonucleotide primers: forward primer (nucleotide residues 2689-2712 of SEQ ID NO: 10) TCTTggatcc ACATTTACTG AATATATTAA GAAT (SEQ ID NO: 17), reverse primer (nucleotide residues 3987-3968 of SEQ ID NO: 10) TTCTgagctc TTACAGTGCC TTTCTCCCC (SEQ ID NO: 14), where the BamHI restriction site in the forward primer sequence and the SacI restriction site in the reverse primer sequence are in lower case characters.

PCR amplified fragments encoding deletion derivatives of the BoNT HC were treated with respective restriction endonucleases and cloned into plasmid pQE-30 in frame with the ATG codon and a 6×His Tag. The three resultant clones thus obtained were designated as pQE-BoNT/A HC88, pQE-BoNT/A HC66 and pQE-BoNT/A HC50, harboring deletion fragments of the BoNT A HC gene encoding 88 kHC, 66 kHC and 50 kHC, respectively. All cloning and expression were performed in E. coli strain BL21 codon plus (DE3)-RIL (obtained from Stratagene, La Jolla, Calif., U.S.A.). All the recombinant clones were confirmed by DNA sequencing.

### Construction of Plasmid Expressing GFP-66 kHC Fusion.

The coding sequence of green fluorescent protein (GFP) was generated by PCR. A DNA fragment encoding 26 kilodalton (26K) GFP protein was amplified using primers having the following nucleotide sequences: forward primer ACATgcatgc ATGAGTAAAG GAGAAGAACT TC (SEQ ID NO: 18), reverse primer CCggtaccCC AGGCCCATTT GTAGAGCTCA TC (SEQ ID NO: 19), where the SphI restriction site in the forward primer sequence and the KpnI restriction site in the reverse primer sequence are in lower case characters. The amplified fragment harboring GFP gene was treated with SphI and KpnI and inserted between the SphI and KpnI restriction sites of plasmid pQE-66 kHC. The resultant plasmid pQE-GFP-66 kHC contained the GFP gene in frame with the ATG codon, 6×His tag and 66 kHC gene.

### Expression and Purification of Recombinant Proteins.

Cultures were grown in Lennox broth at 37° C., with shaking, to an absorbance value at 600 nanometers (A₆₀₀) of 0.6 to 0.8. Isopropyl-beta-D-thiogalactopyranoside (IPTG) was added to 1.0 millimolar (final concentration), and incubation was continued for an additional 5 hours. Bacteria from 1 liter of induced culture were harvested by centrifugation at 4° C. and re-suspended in 20 milliliters of 50 millimolar sodium phosphate buffer (pH. 7.4) with 300 millimolar NaCl. The cell suspension was lysed on ice by sonication, with two pulses of 1 minute each at 75% power, with a model 60 sonic dismembrator (Fisher Scientific, Malvern, Pa., U.S.A.). Lysates were centrifuged at 20000×g for 30 minutes at 4° C. The clarified supernatants were mixed with 2 milliliters of packed nitriletriacetic resin, incubated for one hour at 4° C. on a rotator, and finally poured into a 25-milliliter column.

The column was washed with 30 volumes of washing buffer (50 millimolar sodium phosphate (pH 6.0), 300 millimolar NaCl, 25 millimolar imidazole). Bound proteins were eluted with elution buffer (50 millimolar sodium phosphate (pH 4.5), 300 millimolar NaCl). Purified proteins were analyzed by sodium dodecyl sulfate-polyacrylamide gel electrophoresis (SDS-PAGE) and Western blotting.

### Nicking of BoNT

Botulinum toxin is expressed as a relatively inactive single chain molecule. To become fully active, the toxin must undergo proteolytic processing (“nicking”)” to yield its dichain form. In the laboratory, this is typically accomplished with trypsin.

In order to facilitate the subsequent separation of toxin from the nicking enzyme, TPCK (L-1-tosylamido-2-phenylethyl chloromethyl ketone) treated trypsin cross-linked to 4% beaded agarose was used (Immobilized Trypsin; PIERCE, Rockford, Ill., U.S.A.). The trypsin slurry was washed 3 times with reaction buffer (10 millimolar Sodium Phosphate Buffer, pH 7.5). Toxin was added and incubated with enzyme at room temperature (23° C.) for one hour at a 1:10 ratio of trypsin to toxin. After incubation, the reaction mixture was centrifuged at 10,000 rotations per minute in an Eppendorf tabletop centrifuge for 5 minutes. The supernatant containing “nicked” toxin was collected and stored at −20° C. Alternatively, “nicked” toxin can be separated from the beaded trypsin by filtration through a 0.2 micron centrifugal filter (Schleicher & Schuell Centrex Microfilter Unit) into a clean, sterile tube. A sample of the material is examined by electrophoresis to verify nicking.

### Reduction of BoNT

Dichain toxin consists of a LC (enzymatic portion) and a HC (binding and translocation portion) linked by a disulfide bond. This bond must be reduced (broken) for the light chain to exert its enzymatic activity, the cleavage of proteins responsible for neurotransmitter release. In order to perform in vitro cleavage experiments, native toxin has to be reduced.

Botulinum toxin was reduced by incubating it with dithiothreitol (DTT; Cleland's Reagent) in phosphate buffer at physiological pH (pH 7.2-7.4) or in phosphate buffered saline (PBS). The concentration of DTT typically used was 5 millimolar to 20 millimolar, depending on the experiment. The DTT and toxin reaction mixture was incubated at room temperature (23° C.) for one hour. Toxin reduction was verified by electrophoresis on non-reducing gels.

### Attachment of ¹²⁵I-Bolton Hunter Reagent (MW 387.2) to Purified BoNT and its Fragments

Bolton-Hunter reagent was purchased from PerkinEImer Life Sciences, Inc. (Boston, Mass., U.S.A.). This molecule is supplied with a reactive succinimidyl ester moiety that reacts with primary amines of proteins (e.g., lysine residues). The toxin, HC or its fragments were iodinated using (¹²⁵I)-Bolton Hunter reagent essentially according to manufacturer's instructions. The reaction time was reduced in order to diminish the loss of biological activity of the resulting product. The proteins were labeled to an average specific activity of 500 Curies per millimole or less.

Purified protein (350 micrograms) in borate buffer (pH 7.8; 200 microliters) was added to dried, iodinated ester and reacted on ice for fifteen minutes. The reaction was terminated by addition of 50 microliters of 1 molar glycine in borate buffer for fifteen minutes. The total reaction mixture (250 microliters) and rinse (250 microliters) were loaded onto a SEPHADEX™ G-25 column that was pre-equilibrated with filtration buffer (150 millimolar Na₂HPO₄, 150 millimolar NaCl, 0.1% (w/v) gelatin, pH 7.4). The labeled toxin was eluted with filtration buffer, and 0.5 milliliter fractions were collected. An aliquot (5 microliters) of each fraction was assayed for radioactivity. The labeled toxin peak, which eluted at void volume, was pooled and stored at 3° C. Toxin concentration in the pooled fraction was determined spectrophotometrically at 278 nanometers using the following relationship 1.63 A₂₇₈=1 milligram per milliliter. A portion of this sample was counted in a gamma-counter to quantify the labeled toxin. Sample concentration and associated counts were used to calculate specific activity. Labeled toxin was stored at 3° C.

### Attachment of ALEXA FLUOR® 568 (MW 792) to Purified BoNT

ALEXA FLUOR® 568 is a dye molecule with an absorption (excitation) maximum at 577 nanometers and an emission maximum at 603 nanometers. The dye was purchased from Molecular Probes (Eugene, Oreg., United States of America) and was used according to manufacturer's directions. This molecule is supplied with a reactive succinimidyl ester moiety that reacts with primary amines of proteins (e.g., lysine residues). 0.50 Milliter of a 1.0 milligram per milliliter solution of PBS with purified BoNT A was supplemented with 50 microliters of 1.0 molar sodium bicarbonate, and subsequently added to the dye. The reaction continued with stirring for one hour at room temperature. Hydroxylamine solution (17 microliters) was added, and incubation was continued an additional 30 minutes at room temperature to stop the reaction. The entire reaction volume was then filtered through a gel filtration column equilibrated with and eluted with PBS. The first colored band that eluted from the column (labeled toxin) was collected and stored at 3° C. The amount of labeling was calculated according to the manufacturer's instructions, employing the extinction coefficients of the ALEXA FLUOR® dye and the toxin. The toxin used in transcytosis experiments was labeled with 3 to 7 moles of dye per mole of toxin.

### Transcytosis Assay

Monolayers of polarized epithelial cells are grown on polycarbonate membranes with a 0.4 micrometer pore size in TRANSWELL® (Corning-Costar, Cambridge, Mass., U.S.A.) porous bottom inserts. The TRANSWELL® apparatus permits containment of a product on either the apical or basolateral face of an epithelial cell culture. In the absence of transcytosis of the product across the epithelial cell layer, substantially all of the product is retained on one side of the epithelium by the apparatus. The TRANSWELL® apparatus is therefore useful for assessing transepithelial transcytosis of products.

The cell growth area within each TRANSWELL® insert is equivalent to one square centimeter. Prior to seeding cells, insert membranes were coated with 10 micrograms per square centimeter rat tail type I collagen. Collagen stock solution (6.7 milligrams per milliliter) was prepared in sterile 1% (v/v) acetic acid and stored at 3° C. This collagen stock solution was diluted, as needed, in ice cold 60% (v/v) ethanol, and 150 microliters of the resulting solution containing 10 micrograms of diluted collagen was added to each well.

The collagen solution was allowed to dry at room temperature overnight (about eighteen hours). After drying, the wells were sterilized under UV light for one hour, followed by a pre-incubation with cell culture medium (thirty minute incubation). The pre-incubation medium was removed immediately prior to addition of cells and fresh medium. Cells were plated in the TRANSWELL® apparatus at confluent density. The volumes of medium added were 0.5 milliliter to the upper chamber and 1.0 milliliter to the bottom chamber. Culture medium was changed every two days. The cultures maintained in twelve-well plates were allowed to differentiate a minimum of ten days before use. The integrity of cell monolayers and formation of tight junctions were visualized by monitoring the maintenance of a slightly higher medium meniscus in the inserts as compared to the bottom wells. Formation of tight junctions were confirmed experimentally by assaying the rate of (³H)-inulin diffusion from the top well into the bottom chamber or by measurement of transepithelial resistance across the monolayer.

Transcytosis was assayed by replacement of medium, usually in the top well, with an appropriate volume of medium containing various concentrations of (¹²⁵I)-labeled protein of interest. Transport of radiolabeled protein was monitored by sampling the entire content of opposite wells, which was usually the bottom wells. Aliquots (0.5 milliliter) of the sampled medium were filtered through a SEPHADEX™ G-25 column, and 0.5 milliliter fractions were collected. The amount of radioactivity in the fractions was determined using a gamma counter. The amount of transcytosed protein was normalized and expressed as femtomoles per hour per square centimeter of cultured cell surface. A minimum of two replicates per condition were included in each experiment, and experiments were typically reproduced at least three times.

### Toxicity Testing In vitro toxicity testing.

The toxicity of expressed proteins was bioassayed on mouse phrenic nerve-hemidiaphragm preparations. Tissues were excised and suspended in physiological buffer that was aerated with 95% O₂, 5% CO₂ and maintained at 35° C. The physiological solution had the following composition: 137 millimolar NaCl; 5 millimolar KCl; 1.8 millimolar CaCl₂; 1.0 millimolar MgSO₄; 24 millimolar NaHCO₃; 1.0 millimolar NaH₂PO₄; 11 millimolar D-glucose; and 0.01% (w/v) gelatin. Phrenic nerves were stimulated continuously (1.0 Hertz; 0.1-0.3 millisecond duration), and muscle twitch was recorded. Toxin-induced paralysis was measured as a 50% reduction in muscle twitch response to neurogenic stimulation.

### In Vivo Toxicity Testing.

The toxicity of expressed proteins was tested by administering the proteins to laboratory mice. Proteins purified by elution from a histidine affinity resin or GST affinity resin were diluted in PBS including 1 milligram per milliliter bovine serum albumin (BSA) and injected intraperitoneally (i.p.) to mice. The recombinant proteins were administered in a 100 microliters aliquot of PBS-BSA at concentrations of 1 to 100 micrograms per animal (average weight of approximately 25 grams). Animals were monitored for varying lengths of time to detect any non-specific toxicity.

### Surgical Administration of Toxin or Fragments into Stomach or Intestine

Swiss-Webster mice (female, 25 grams each), were purchased from Ace Animals (Boyertown, Pa., U.S.A.) and allowed unrestricted access to food and water.

Pre-operative protocol involved fasting animals for eighteen hours prior to surgery, although allowing free access to water. Pre-operative preparation also included shaving the abdominal area and administering a prophylactic, subcutaneous dose of gentamicin sulfate (6 milligrams per kilogram body weight. (available from Fujusawa USA, Inc., Deerfield, Ill., U.S.A.). On the day of surgery, animals were transferred to a veterinary procedure room, and all subsequent steps were performed in an aseptic surgical environment.

Animals were anesthetized by administration of Isoflurane (ISO-THESIA™, Abbott Laboratories North, Chicago, Ill., U.S.A.) and oxygen, and this same inhalation anesthetic was administered throughout surgery. An abdominal laparotomy (about 1.5 to 2.5 centimeters, depending on the size of the mouse) was performed, and either the stomach or the small intestine immediately proximal to the stomach was partially externalized. If required by protocol, a ligature was placed immediately above (proximal to the stomach) the pyloric sphincter using 3-0 PROLENE™ (polypropylene suture, Ethicon, Inc., Somerville, N.J., U.S.A.). Care was taken so that this ligature was sufficient to prevent flow of stomach juices into the intestine (or reverse flow of intestinal contents into the stomach), but not sufficient to cause mechanical damage to the tissues involved. Neurotoxin was administered through a 1 milliliter tuberculin syringe with a 0.5 inch, 27 gauge needle. Injection volumes were kept constant at 100 microliters per animal regardless of site of administration (stomach or intestine). For all injections, the vehicle consisted of sterile Dulbecco's PBS (pH 7.4) with 1 milligram BSA per milliliter. Neurotoxin was administered into the lumen of the stomach by injection through the stomach wall at the greater curvature, with care to avoid the gastro-epiploic vessels. Neurotoxin was administered into the lumen of the small intestine by oblique insertion of the needle parallel to the segment and always in a direction away from the stomach. The time of injection was recorded.

After administration of neurotoxin, organs were gently repositioned and the incision in the abdominal muscle was sutured using 3-0 PROLENE™. The skin was closed using several small wound clips, after which animals received an analgesic injection of buprenorphine hydrochloride (2 milligrams per kilogram body weight subcutaneously; BUPRENEX® injectable, Reckitt & Colman Pharmaceuticals, Inc., Richmond, Va., United States of America) and another dose of Gentamicin.

The surgical procedure lasted approximately fifteen minutes per animal, and suspension of anesthesia resulted in full recovery within ten to fifteen minutes. Animals were then transferred to the laboratory where they were monitored for assay endpoint. The time of death was recorded, and total elapsed minutes from time of injection to time of death were calculated.

### Method for Immunization

Toxin variants that retain the ability to bind and cross gut and airway epithelial cells were tested for their abilities to evoke immunity following oral and/or intranasal administration. To be judged worthy of further consideration, a potential oral or inhalation vaccine had to evoke protection against at least 1000 MLD₅₀ of the parent toxin. Specific pathogen-free female Swiss-Webster mice were used in this work.

For subcutaneous (s.c.) immunization, animals received 1-20 micrograms protein in 0.1 milliliter of PBS. Four doses were given at fourteen day intervals. The mice were bled seven days after the second, third and fourth immunization and analyzed by immunoblotting for immunoreactivity to toxin variants. Mice were challenged with at least 1×10³ MLD₅₀ of parent toxin via the intraperitoneal (i.p.) route. Prior to injection, toxin was diluted in PBS containing 1% (w/v) gelatin. Mice were challenged fourteen days following their final vaccination, and untreated mice were used as controls.

For intranasal immunization, mice received 1-20 micrograms of protein suspended in 20 microliters of PBS. Mice were lightly anesthetized with isoflurane (ISO-THESIA™, Abbott Laboratories North, Chicago, Ill., U.S.A.). Protein was administered by a single application of 10 to 20 microliters of the suspension to the nares. The heads of animals were maintained in an upright position to minimize drainage into the posterior pharynx. Five doses were given at seven day intervals. The mice were bled seven days after the third, fourth, and fifth immunization and the specimens were analyzed by immunoblotting for immunoreactivity to toxin variants. Mice were challenged with at least 1×10³ MLD₅₀ of parent toxin via the i.p. route ten days following their final vaccination.

Oral immunizations were performed by inoculation of 1-20 micrograms of protein suspended in 100 microliters of PBS. Mice were lightly anesthetized with isoflurane (ISO-THESIA™, Abbott Laboratories North, Chicago, Ill., United States of America), and protein was administered by a single application via a feeding tube. Five doses were given at seven day intervals. The mice were bled seven days after the third, fourth, and fifth immunization, and specimens were analyzed by immunoblotting for immunoreactivity to toxin variants. Mice were challenged with at least 1×10³ MLD₅₀ of parent toxin via the i.p. route ten days following their final vaccination.

### Oral Immunization

Swiss-Webster mice (female, 20-25 grams each) were purchased from Ace Animals (Boyertown, Pa., U.S.A.) and allowed unrestricted access to food and water. The mice were immunized per os (p.o.). For p.o. administration, each animal was fed 4 micrograms of protein suspended in 0.2 milliliter elution buffer administered through an intragastric feeding needle. Mice were immunized on day 0, and boosters were given on days 14, 28, and 42. Samples of serum from identically immunized mice were collected and pooled on days 21, 35, and 49. For collection of serum, mice were bled with capillary tubes at the retro-orbital plexus while under isoflurane anesthesia.

Sera from immunized or control mice were assayed for antibodies using immunoblot analysis. Recombinant antigen (holotoxin or fragment; 0.1 microgram/lane) was separated by SDS-PAGE and transferred to nitrocellulose membranes. Membranes were blocked with 5% (w/v) non-fat powdered milk in Tris-buffered saline (TBS), cut into strips and processed for detection of immunoreactive proteins using various serum samples.

Primary incubations were performed overnight (eighteen hours) at room temperature with 1:1000 diluted serum. A secondary horseradish peroxidase-labeled anti-mouse IgG was used at 1:10000 dilution for one hour at room temperature. After extensive washing, membranes were developed using enhanced chemiluminescent reagents (ECL™, Amersham Biosciences, Piscataway, N.J., U.S.A.).

### Enzyme Linked Immunosorbent Assays (ELISA)

ELISA was performed as described by Siegel, with only minor modifications. Highly purified (>95%) BoNT A was diluted to 5 micrograms per milliliter in phosphate-buffered saline, pH 7.4, and then added to microtiter plates (100 microliters/well) that were incubated at 4° C. overnight in a sealed container.

One percent BSA in TBS with 0.1% (v/v) TWEEN™ 20 was used to block nonspecific binding. Serum samples were initially diluted 1:30 and then serially diluted fourfold for a total of seven dilutions (1:30 to 1:122, 880). Diluted sera were added in duplicate to toxin-coated wells (100 microliters per well). The secondary antibody was alkaline phosphatase-conjugated goat anti-human or anti-mouse IgA or IgG diluted 1:1000. The primary and secondary antibodies were incubated for sixty minutes at 37° C. p-Nitrophenyl phosphate (100 microliters per well) was added as a substrate. Plates were incubated at room temperature for 30 minutes, and absorbance was measured with a microplate reader at 405 nanometers. ELISA titers were defined as the reciprocal of the highest serum dilution giving an absorbance of 0.2 (absorbance units) above background.

### Capture ELISA for Detection of Recombinant BoNT Fragments in Mouse Plasma after Intranasal Administration

Microtiter plates were coated overnight at 4° C. with 100 microliters per well of a solution containing 1:1000 diluted rabbit anti-botulinum toxoid A in coating buffer (0.1 molar Na₂CO₃, pH 9.6). The remaining sites of absorption were then blocked by the addition of 1% (w/v) BSA in wash buffer (20 millimolar TBS, 0.1% (v/v) TWEEN™ 20, pH 7.6) for one hour at 37° C. The plates were then washed 4 times with wash buffer. Standard curves were prepared by diluting antigen with the appropriate volumes of assay buffer (20 millimolar TBS, pH 7.6) or the appropriate mouse serum. Standards and serum samples (100 microliters per well) were incubated for one hour at 37° C. and the plates were washed as described above. The 1:500 diluted mouse anti-BoNT A HC was added and incubated for one hour at 37° C. The plates were washed three times with wash buffer, and 100 microliters alkaline phosphatase-labeled goat anti-mouse IgG conjugate diluted (1:5000) in wash buffer was added and incubated for one hour at 37° C. After washing the plates, 100 microliters of substrate solution (p-nitrophenyl phosphate) in glycine buffer, pH 10.4 (0.1 molar glycine, 1 millimolar MgCl₂, and 1 millimolar ZnCl₂), was added to each well. The reaction was stopped after 30 minutes incubation at room temperature, and the absorbance was measured at 405 nanometers.

Cell types used in the examples include T-84 human gut epithelial cells (T-84 and Caco-2 cells), human pulmonary epithelial cells (Calu-3), and Madin-Darby canine kidney epithelial cells (MDCK cells). Cells of these types are available commercially, for example from American Tissue Culture Collection (ATCC), Manassas, Va., U.S.A. Primary cultures of rat epithelial alveolar cells were also used.

### Example 1

### Detection of Transcytosis of BoNT A from the Apical Surface of T-84 Cells to the Basolateral Side of the Cells by Western Blotting

This experiment was carried out using native BoNT A and human gut epithelial cells (“T-84 cells”). Transcytosis was assayed in T-84 cell cultures using a TRANSWELL® apparatus assay system. Assay was initiated by addition of 1×10⁻⁸ molar native, purified, BoNT A to the upper chamber. Cultures were subsequently incubated for eighteen hours at 37° C. At the end of each experiment, contents of three basal chambers per condition were collected and concentrated in a CENTRICON™ micro-concentrator. The resulting solution was run on 7.5% (w/v) SDS-PAGE and subsequently transferred to nitrocellulose membranes. The identity and molecular weight of the transcytosed molecule was confirmed by Western blotting with anti-HC antibody.

A Western blot demonstrated that pre-transcytosis control (BoNT A) and BoNT A transcytosed through T-84 cells (i.e., collected from basal chamber) had approximately the same size. Thus, not only did the BoNT A efficiently cross T-84 cells, but the molecular weight of the molecule was unaltered, indicating that the mechanism by which transcytosis was accomplished did not result in modification of BoNT.

### Example 2

### Western Blot of Apical to Basolateral Transcytosed BoNT A in MDCK Cells

This experiment was carried out using BoNT A and Madin-Darby Canine Kidney Cells (“MDCK cells”). Transcytosis was assayed in MDCK cell cultures using a TRANSWELL® apparatus assay system. Assay was initiated by addition of 1×10⁻⁸ molar native, purified, BoNT A to the upper chamber. Cultures were subsequently incubated for eighteen hours at 37° C. At the end of each experiment, contents of three basal chambers per condition were collected and concentrated in a CENTRICON™ micro-concentrator. The resulting solution was run on 7.5% (w/v) SDS-PAGE and subsequently transferred to nitrocellulose membranes. The identity and molecular weight of the transcytosed molecule was confirmed by Western blotting with anti-HC antibody.

The Western blot indicated that native BoNT A is poorly bound, internalized, transcytosed, and released by MDCK cells. BoNT A was not detected in medium collected from the basolateral side of cells, leading to the conclusion that BoNT A did not efficiently cross MDCK cells.

### Example 3

### Apical to Basolateral Transcytosis of BoNT A in T-84 Cells

Transcytosis of BoNT A linked to ¹²⁵I at lysine residues was assayed in human gut epithelial cells (“T-84”) cell cultures using a TRANSWELL® apparatus assay system. Assay was initiated by addition of 1×10⁻⁸ molar (¹²⁵I)-BoNT A to the upper chamber. Cultures were subsequently incubated for eighteen hours at 37° C. At the end of each experiment contents of the basal chamber were collected and gel filtered. Void volume fractions were assayed for radioactivity and the toxin peak was summed to determine total counts. The amount of transcytosis was calculated based on the specific activity of labeled BoNT A.

The results indicate that BoNT A linked to ¹²⁵It was transported from the apical to the basolateral side of cells. It efficiently crossed T-84 cells at a transcytosis rate of 11.29±0.30 femtomoles per hour per square centimeter.

There are three major conclusions that stem from the experimental results. First, the purified botulinum neurotoxin is bound, internalized, transcytosed, and released by differentiated, polarized human gut epithelial cells. Second, modification of lysine residues by attachment of ¹²⁵I does not alter the ability of the holotoxin to display these properties. Third, the BoNT A is capable of transporting the ¹²⁵I-Bolton-Hunter reagent from the apical to the basolateral side of human gut epithelial cells.

### Example 4

### Basolateral to Apical Transcytosis of BoNT A in T-84 Cells

Transcytosis of BoNT A linked to ¹²⁵I at lysine residues was assayed in T-84 cell cultures using a TRANSWELL® apparatus assay system. Assay was initiated by addition of 1×10⁻⁸ molar (¹²⁵I)-BoNT A to the lower chamber. Cultures were subsequently incubated for eighteen hours at 37° C. At the end of each experiment contents of the upper chamber were collected and gel filtered. Void volume fractions were assayed for radioactivity and the toxin peak was summed to determine total counts. The amount of transcytosis was calculated based on the specific activity of labeled BoNT A.

The results show that BoNT A linked to ¹²⁵I was transported from the basolateral to the apical side of cells. It efficiently crossed T-84 cells, and the rate of transcytosis was quantified at 8.98±0.20 femtomoles per hour per square centimeter.

There are three major conclusions that stem from the experimental results. First, the purified botulinum neurotoxin is bound, internalized, transcytosed, and released by differentiated, polarized human gut epithelial cells. This process is somewhat less efficient in the basolateral to apical direction than in the reverse direction. Second, modification of lysine residues does not alter the ability of the holotoxin to display these properties. Third, the BoNT A is capable of transporting the ¹²⁵I-Bolton-Hunter reagent from the basolateral to the apical side of human gut epithelial cells.

### Example 5

### Apical to Basolateral Transcytosis of BoNT A in Caco-2 Cells

Transcytosis of BoNT A linked to ¹²⁵I at lysine residues was assayed in Caco-2 cell cultures using a TRANSWELL® apparatus assay system. Assay was initiated by addition of 1×10⁻⁸ molar (¹²⁵I)-BoNT A to the upper chamber. Cultures were subsequently incubated for eighteen hours at 37° C. At the end of each experiment, contents of the basal chamber were collected and gel filtered. Void volume fractions were assayed for radioactivity and the toxin peak was summed to determine total counts. The amount of transcytosis was calculated based on the specific activity of labeled toxin.

The results show that purified neurotoxin was transported from the apical to the basolateral side of cells. BoNT A efficiently crossed Caco-2 cells. The rate of transcytosis was quantified at 8.42±0.49 femtomoles per hour per square centimeter.

There are four major conclusions that stem from the experimental results. First, the purified botulinum neurotoxin is bound, internalized, transcytosed, and released by differentiated, polarized human gut epithelial cells. Second, modification of lysine residues does not alter the ability of the holotoxin to display these properties. Third, the BoNT A is capable of transporting the ¹²⁵I-Bolton-Hunter reagent from the apical to the basolateral side of human gut epithelial cells. Fourth, the HC is capable of transporting more than one molecule (LC & Bolton-Hunter reagent) at a time across human gut epithelial cells.

### Example 6

### Apical to Basolateral Transcytosis of BoNT A in MDCK Cells

Transcytosis of BoNT A linked to ¹²⁵I at the lysine residues was assayed in Madin-Darby Canine Kidney (“MDCK”) cell cultures using a TRANSWELL® apparatus assay system. Assay was initiated by addition of 1×10⁻⁸ molar (¹²⁵I)-BoNT A to the upper chamber. Cultures were subsequently incubated for eighteen hours at 37° C. At the end of each experiment, contents of the basal chamber were collected and gel filtered. Void volume fractions were assayed for radioactivity and the toxin peak was summed to determine total counts. The amount of transcytosis was calculated based on the specific activity of labeled BoNT A.

The results show that BoNT A was poorly transported from the apical to the basolateral side of cells. Purified neurotoxin did not efficiently cross MDCK cells, as evidenced by the rate of transcytosis of 0.05±0.01 femtomoles per hour per square centimeter.

The BoNT A is poorly bound, internalized, transcytosed, and released by polarized MDCK cells.

### Example 7

### Apical to Basolateral Transcytosis of Un-Nicked Botulinum Neurotoxin Serotype B in T-84 Cells

Transcytosis of un-nicked botulinum neurotoxin linked to ¹²⁵I at lysine residues was assayed in T-84 cell cultures using a TRANSWELL® apparatus assay system. Assay was initiated by addition of 1×10⁻⁸ molar (¹²⁵I)-uBoNT B to the upper chamber. Cultures were subsequently incubated for eighteen hours at 37° C. At the end of each experiment contents of the basal chamber were collected and gel filtered. Void volume fractions were assayed for radioactivity and the toxin peak was summed to determine total counts. The amount of transcytosis was calculated based on the specific activity of labeled uBoNT.

The results show that uBoNT was transported from the apical to the basolateral side of cells. It efficiently crossed T-84 cells at a rate of 9.01±0.44 femtomoles per hour per square centimeter.

There are four major conclusions that stem from the experimental results. First, the purified, un-nicked botulinum neurotoxin, serotype B, is bound, internalized, transcytosed, and released by differentiated, polarized human gut epithelial cells. Second, modification of lysine residues does not alter the ability of the uBoNT to display these properties. Third, the uBoNT is capable of transporting the ¹²⁵I-Bolton-Hunter reagent from the apical to the basolateral side of human gut epithelial cells. Fourth, the HC of uBoNT is capable of transporting more than one molecule (LC portion & Bolton-Hunter reagent) at a time across human gut epithelial cells.

### Example 8

### Apical to Basolateral Transcytosis of Un-Nicked Botulinum Neurotoxin Serotype B in MDCK Cells

Transcytosis of un-nicked botulinum neurotoxin linked to ¹²⁵I at lysine residues was assayed in Madin-Darby Canine Kidney (“MDCK”) cell cultures using a TRANSWELL® apparatus assay system. Assay was initiated by addition of 1×10⁻⁸ molar (¹²⁵I)-Botulinum toxin type B to the upper chamber. Cultures were subsequently incubated for eighteen hours at 37° C. At the end of each experiment, contents of the basal chamber were collected and gel filtered. Void volume fractions were assayed for radioactivity and the toxin peak was summed to determine total counts. The amount of transcytosis was calculated based on the specific activity of labeled toxin.

The results show that uBoNT B was poorly transported from the apical to the basolateral side of cells. It did not efficiently cross MDCK cells (0.05±0.00 femtomoles per hour per square centimeter). The uBoNT serotype B is poorly bound, internalized, transcytosed, and released by polarized MDCK cells.

### Example 9

### Basolateral to Apical Transcytosis of Un-Nicked Botulinum Neurotoxin Serotype B in T-84 Cells

Transcytosis of un-nicked botulinum neurotoxin linked to ¹²⁵I at lysine residues was assayed in T-84 cell cultures using a TRANSWELL® apparatus assay system. Assay was initiated by addition of 1×10⁻⁸ molar (¹²⁵I)-uBoNT B to the lower chamber. Cultures were subsequently incubated for eighteen hours at 37° C. At the end of each experiment contents of the apical chamber were collected and gel filtered. Void volume fractions were assayed for radioactivity and the toxin peak was summed to determine total counts. The amount of transcytosis was calculated based on the specific activity of labeled uBoNT.

The results show that uBoNT was transported from the basolateral to the apical side of cells. uBoNT efficiently crossed T-84 cells at a rate of 4.48±0.00 femtomoles per hour per square centimeter.

Several conclusions may be drawn. First, uBoNT is bound, internalized, transcytosed, and released by differentiated, polarized human gut epithelial cells. Second, modification of lysine residues does not alter the ability of uBoNT to display these properties. Third, the uBoNT is capable of transporting the ¹²⁵-Bolton-Hunter reagent from the basolateral to the apical side of human gut epithelial cells. Fourth, the HC of uBoNT is capable of transporting more than one molecule (LC & Bolton-Hunter reagent) at a time across human gut epithelial cells.

### Example 10

### Basolateral to Apical Transcytosis of Un-Nicked Botulinum Neurotoxin Serotype B in MDCK Cells

Transcytosis of un-nicked botulinum neurotoxin linked to ¹²⁵I at lysine residues was assayed in Madin-Darby Canine Kidney (“MDCK”) cell cultures using a TRANSWELL® apparatus assay system. Assay was initiated by addition of 1×10⁻⁸ molar (¹²⁵I)-uBoNT to the lower chamber. Cultures were subsequently incubated for eighteen hours at 37° C. At the end of each experiment, contents of the apical chamber were collected and gel filtered. Void volume fractions were assayed for radioactivity and the toxin peak was summed to determine total counts. The amount of transcytosis was calculated based on the specific activity of labeled uBoNT.

The results show that purified uBoNT was poorly transported from the basolateral to the apical side of cells. Purified uBoNT did not efficiently cross MDCK cells (0.05±0.00 femtomoles per hour per square centimeter).

The purified uBoNT B is poorly bound, internalized, transcytosed, and released by polarized MDCK cells.

### Example 11

### Apical to Basolateral Transcytosis of Nicked Botulinum Neurotoxin Serotype B in T-84 Cells

Transcytosis of nicked BoNT B linked to ¹²⁵I at lysine residues was assayed in T-84 cell cultures using a TRANSWELL® apparatus assay system. Assay was initiated by addition of 1×10⁻⁸ molar (¹²⁵I)-nBoNT B to the upper chamber. Cultures were subsequently incubated for eighteen hours at 37° C. At the end of each experiment contents of the basal chamber were collected and gel filtered. Void volume fractions were assayed for radioactivity and the toxin peak was summed to determine total counts. The amount of transcytosis was calculated based on the specific activity of labeled nBoNT B.

The results show that purified and nicked neurotoxin was transported from the apical to the basolateral side of cells. Purified neurotoxin efficiently crossed T-84 cells at a rate of 6.57±0.07 femtomoles per hour per square centimeter.

Several conclusions may be drawn. First, the purified, nBoNT B is bound, internalized, transcytosed, and released by differentiated, polarized human gut epithelial cells. Second, modification of lysine residues does not alter the ability of the holotoxin to display these properties. Third, the nBoNT B is capable of transporting the ¹²⁵I-Bolton-Hunter reagent from the apical to the basolateral side of human gut epithelial cells. Fourth, the HC of nBoNT B is capable of transporting more than one molecule (LC & Bolton-Hunter reagent) at a time across human gut epithelial cells.

### Example 12

### Apical to Basolateral Transcytosis of Nicked Botulinum Neurotoxin Serotype B in MDCK Cells

Transcytosis of nBoNT B linked to ¹²⁵I at lysine residues was assayed in Madin-Darby Canine Kidney (“MDCK”) cell cultures using a TRANSWELL® apparatus assay system. Assay was initiated by addition of 1×10⁻⁸ molar (¹²⁵I)-nBoNT B to the upper chamber. Cultures were subsequently incubated for eighteen hours at 37° C. At the end of each experiment, contents of the basal chamber were collected and gel filtered. Void volume fractions were assayed for radioactivity and the toxin peak was summed to determine total counts. The amount of transcytosis was calculated based on the specific activity of labeled nBoNT.

The results show that purified nBoNT was poorly transported from the apical to the basolateral side of cells. Purified nBoNT did not efficiently cross MDCK cells (0.05±0.00 femtomoles per hour per square centimeter).

The purified nBoNT B is poorly bound, internalized, transcytosed, and released by polarized MDCK cells.

### Example 13

### Basolateral to Apical Transcytosis of Nicked Botulinum Neurotoxin Serotype B in t-84 Cells

Transcytosis of nBoNT B linked to ¹²⁵I at the lysine residues was assayed in human gut epithelial (“T-84”) cell cultures using a TRANSWELL® apparatus assay system. Assay was initiated by addition of 1×10⁻⁸ molar (¹²⁵I)-nBoNT B to the lower chamber. Cultures were subsequently incubated for eighteen hours at 37° C. At the end of each experiment contents of the apical chamber were collected and gel filtered. Void volume fractions were assayed for radioactivity and the toxin peak was summed to determine total counts. The amount of transcytosis was calculated based on the specific activity of labeled nBoNT B.

The results show that nBoNT was transported from the basolateral to the apical side of cells. Purified nBoNT efficiently crossed T-84 cells at a rate of 5.54±0.00 femtomoles per hour per square centimeter.

Several conclusions may be drawn. First, the purified nBoNT B is bound, internalized, transcytosed, and released by differentiated, polarized human gut epithelial cells. Second, modification of lysine residues does not alter the ability of the nBoNT B to display these properties. Third, nBoNT B is capable of transporting the ¹²⁵I-Bolton-Hunter reagent from the basolateral to the apical side of human gut epithelial cells. Fourth, the HC of nBoNT B is capable of transporting more than one molecule (LC & Bolton-Hunter reagent) at a time across human gut epithelial cells.

### Example 14

### Basolateral to Apical Transcytosis of Nicked Botulinum Neurotoxin Serotype B in MDCK Cells

Transcytosis of nBoNT B linked to ¹²⁵I at lysine residues was assayed in Madin-Darby Canine Kidney (“MDCK”) cell cultures using a TRANSWELL® apparatus assay system. Assay was initiated by addition of 1±10⁻⁸ molar (¹²⁵I)-BoNT B to the lower chamber. Cultures were subsequently incubated for eighteen hours at 37° C. At the end of each experiment, contents of the apical chamber were collected and gel filtered. Void volume fractions were assayed for radioactivity and the toxin peak was summed to determine total counts. The amount of transcytosis was calculated based on the specific activity of labeled nBoNT.

The results show that purified nBoNT B was poorly transported from the basolateral to the apical side of cells. Purified nBoNT B did not efficiently cross MDCK cells (0.05±0.00 femtomoles per hour per square centimeter). The purified nBoNT B is poorly bound, internalized, transcytosed, and released by polarized MDCK epithelial cells.

### Example 15

### Western Blot of Apical to Basolateral Transcytosed HC in T-84 Cells

Transcytosis of HC, serotype A, was assayed in human gut epithelial (“T-84”) cell cultures using a TRANSWELL® apparatus assay system. Assay was initiated by addition of 1×10⁻⁸ molar HC to the upper chamber. Cultures were subsequently incubated for eighteen hours at 37° C. At the end of each experiment, contents of three basal chambers per condition were collected and concentrated in a CENTRICON™ micro-concentrator. The resulting solution was run on 7.5% (w/v) SDS-PAGE and subsequently transferred to nitrocellulose membranes. The identity and molecular weight of the transcytosed molecule was confirmed by Western blotting with anti-HC antibody.

The results verify that the HC was transported from the apical to the basolateral side of cells. Not only did HC efficiently cross T-84 cells, but the molecular weight of the molecule was unaltered.

There are two major conclusions that stem from the experimental results. First, the HC of botulinum neurotoxin is bound, internalized, transcytosed, and released by differentiated, polarized human gut epithelial cells. Second, after transcytosis, the molecular size of the native HC released on the basolateral side remains unchanged, leading to the conclusion that the process of transcytosis does not alter the physical structure of the HC.

### Example 16

### Western Blot of Apical to Basolateral Transcytosed HC in MDCK Cells

Transcytosis of HC, serotype A, was assayed in Madin-Darby Canine Kidney (“MDCK”) cell cultures using a TRANSWELL® apparatus assay system. Assay was initiated by addition of 1×10⁻⁸ molar HC to the upper chamber. Cultures were subsequently incubated for eighteen hours at 37° C. At the end of each experiment, contents of three basal chambers per condition were collected and concentrated in a CENTRICON™ micro-concentrator. The resulting solution was run on 7.5% (w/v) SDS-PAGE and subsequently transferred to nitrocellulose membranes. The identity and molecular weight of the transcytosed molecule was confirmed by Western blotting with anti-HC antibody.

The results show that the HC was not detected in medium collected from the basolateral side of cells. Thus, HC did not efficiently cross MDCK cells. The HC of botulinum neurotoxin is poorly bound, internalized, transcytosed, and released by polarized MDCK cells.

### Example 17

### Apical to Basolateral Transcytosis of HC in T-84 Cells

Transcytosis of HC, serotype A, linked to ¹²⁵¹ at lysine residues was assayed in human gut epithelial (“T-84”) cell cultures using a TRANSWELL® apparatus assay system. Assay was initiated by addition of 1×10⁻⁸ molar (¹²⁵I)HC to the upper chamber. Cultures were subsequently incubated for eighteen hours at 37° C. At the end of each experiment, contents of the basal chamber were collected and gel filtered. Void volume fractions were assayed for radioactivity and the toxin peak was summed to determine total counts. The amount of transcytosis was calculated based on the specific activity of labeled HC.

The results show that the HC was transported from the apical to the basolateral side of cells. Not only did HC efficiently cross T-84 cells, but the rate of transcytosis (7.10±0.00 femtomoles per hour per square centimeter) was comparable to the rate of transcytosis (11.34 femtomoles per hour per square centimeter) for purified BoNT A.

There are three major conclusions that may be drawn. First, the HC, serotype A, is bound, internalized, transcytosed, and released by differentiated, polarized human gut epithelial cells. Second, modification of lysine residues does not alter the ability of the HC to display these properties. Third, the HC is capable of transporting the ¹²⁵I-Bolton-Hunter reagent from the apical to the basolateral side of human gut epithelial cells.

### Example 18

### Apical to Basolateral Transcytosis of BoNT A HC in MDCK Cells

Transcytosis of HC, serotype A, linked to ¹²⁵I at lysine residues was assayed in Madin-Darby Canine Kidney (“MDCK”) cell cultures using a TRANSWELL® apparatus assay system. Assay was initiated by addition of 1×10⁻⁸ molar (¹²⁵I)-HC to the upper chamber. Cultures were subsequently incubated for eighteen hours at 37° C. At the end of each experiment, contents of the basal chamber were collected and gel filtered. Void volume fractions were assayed for radioactivity and the toxin peak was summed to determine total counts. The amount of transcytosis was calculated based on the specific activity of labeled HC.

The results show that the HC was poorly transported from the apical to the basolateral side of cells. HC did not efficiently cross MDCK cells, as evidenced by the rate of transcytosis of 0.06±0.00 femtomoles per hour per square centimeter. The HC, serotype A, is poorly bound, internalized, transcytosed, and released by polarized kidney epithelial cells.

### Example 19

### Western Blot of Apical to Basolateral Transcytosed 66 kHC in T-84 Cells

Transcytosis of 66 kHC, serotype A, was assayed in human gut epithelial (“T-84”) cell cultures using a TRANSWELL® apparatus assay system. Assay was initiated by addition of 1×10⁻⁸ molar 66 kHC to the upper chamber. Cultures were subsequently incubated for eighteen hours at 37° C. At the end of each experiment, contents of three basal chambers per condition were collected and concentrated in a CENTRICON™ micro-concentrator. The resulting solution was run on 7.5% (w/v) SDS-PAGE and subsequently transferred to nitrocellulose membranes. The identity and molecular weight of the transcytosed molecule was confirmed by Western blotting with anti-HC antibody.

The results verify that 66 kHC was transported from the apical to the basolateral side of cells. Not only did 66 kHC efficiently cross T-84 cells, but the molecular weight of the molecule was unaltered.

There are three major conclusions that may be drawn from these results, as follows: First, 66 kHC is bound, internalized, transcytosed, and released by differentiated, polarized human gut epithelial cells. Second, after transcytosis, the molecular size of 66 kHC released on the basolateral side remains unchanged. Third, 66 kHC is capable of transporting a 6×-histidine tag from the apical to the basolateral side of human gut epithelial cells.

### Example 20

### Western Blot of Apical to Basolateral Transcytosed 66 kHC in MDCK Cells

Transcytosis of 66 kHC, serotype A, was assayed in Madin-Darby Canine Kidney (“MDCK”) cell cultures using a TRANSWELL® apparatus assay system. Assay was initiated by addition of 1×10⁻⁸ molar 66 kHC to the upper chamber. Cultures were subsequently incubated for eighteen hours at 37° C. At the end of each experiment, contents of three basal chambers per condition were collected and concentrated in a CENTRICON™ micro-concentrator. The resulting solution was run on 7.5% (w/v) SDS-PAGE and subsequently transferred to nitrocellulose membranes. The identity and molecular weight of the transcytosed molecule was confirmed by Western blotting with anti-HC antibody.

The results show that 66 kHC was not detected in medium collected from the basolateral side of cells. Thus, 66 kHC did not efficiently cross MDCK cells. 66 kHC is poorly bound, internalized, transcytosed, and released by polarized MDCK cells.

### Example 21

### Western Blot of Apical to Basolateral Transcytosed 50 kHC in T-84 Cells

Transcytosis of 50 kHC, serotype A, was assayed in human gut epithelial (“T-84”) cell cultures using a TRANSWELL® apparatus assay system. Assay was initiated by addition of 1×10⁻⁸ molar 50 kHC to the upper chamber. Cultures were subsequently incubated for eighteen hours at 37° C. At the end of each experiment, contents of three basal chambers per condition were collected and concentrated in a CENTRICON™ micro-concentrator. The resulting solution was run on 7.5% (w/v) SDS-PAGE and subsequently transferred to nitrocellulose membranes. The identity and molecular weight of the transcytosed molecule was confirmed by Western blotting with anti-HC antibody.

The results verify that the 50 kHC fragment was transported from the apical to the basolateral side of cells. Not only did 50 kHC efficiently cross T-84 cells, but the molecular weight of the molecule was unaltered.

There are three major conclusions that may be drawn from the experimental results. First, 50 kHC is bound, internalized, transcytosed, and released by differentiated, polarized human gut epithelial cells. Second, after transcytosis, the molecular size of the 50 kHC fragment released on the basolateral side remains unchanged. Third, the 50 kHC fragment is capable of transporting a 6×-histidine tag from the apical to the basolateral side of human gut epithelial cells.

### Example 22

### Western Blot of Apical to Basolateral Transcytosed 50 kHC in MDCK Cells

Transcytosis of 50 kHC, serotype A, was assayed in Madin-Darby Canine Kidney (“MDCK”) cell cultures using a TRANSWELL® apparatus assay system. Assay was initiated by addition of 1×10⁻⁸ molar 50 kHC to the upper chamber. Cultures were subsequently incubated for eighteen hours at 37° C. At the end of each experiment, contents of three basal chambers per condition were collected and concentrated in a CENTRICON™ micro-concentrator. The resulting solution was run on 7.5% (w/v) SDS-PAGE and subsequently transferred to nitrocellulose membranes. The identity and molecular weight of the transcytosed molecule was confirmed by Western blotting with anti-HC antibody.

The results show that 50 kHC was not detected in medium collected from the basolateral side of cells. Thus, 50 kHC did not efficiently cross MDCK cells. Thus, 50 kHC of botulinum neurotoxin is poorly bound, internalized, transcytosed, and released by polarized MDCK cells.

### Example 23

### Apical to Basolateral Transcytosis of 50 kHC in T-84 Cells

Transcytosis of 50 kHC, serotype A, linked to ¹²⁵I at lysine residues was assayed in human gut epithelial (“T-84”) cell cultures using a TRANSWELL® apparatus assay system. Assay was initiated by addition of 1×10⁻⁸ molar (¹²⁵I)-50 kHC to the upper chamber. Cultures were subsequently incubated for eighteen hours at 37° C. At the end of each experiment, contents of the basal chamber were collected and gel filtered. Void volume fractions were assayed for radioactivity and the toxin peak was summed to determine total counts. The amount of transcytosis was calculated based on the specific activity of labeled 50 kHC.

The results show that 50 kHC was transported from the apical to the basolateral side of cells. Not only did 50 kHC efficiently cross T-84 cells, but the rate of transcytosis (13.97±0.00 femtomoles per hour per square centimeter) was comparable to the rate of transcytosis (11.34 femtomoles per hour per square centimeter) for purified BoNT A.

There are five major conclusions that may be drawn from the experimental results. First, the 50 kHC fragment of botulinum neurotoxin is bound, internalized, transcytosed, and released by differentiated, polarized human gut epithelial cells. Second, modification of lysine residues does not alter the ability of the 50 kHC fragment to display these properties. Third, 50 kHC is capable of transporting the ¹²⁵I-Bolton-Hunter reagent from the apical to the basolateral side of human gut epithelial cells. Fourth, the 50 kHC fragment is capable of transporting a 6×-histidine tag from the apical to the basolateral side of human gut epithelial cells. Fifth, the 50 kHC fragment is capable of transporting more than one molecule (polyhistidine tag & Bolton-Hunter reagent) at a time across human gut epithelial cells.

### Example 24

### Apical to Basolateral Transcytosis of 50 kHC in MDCK Cells

Transcytosis of 50 kHC, serotype A, linked to ¹²⁵I at lysine residues was assayed in Madin-Darby Canine Kidney (“MDCK”) cell cultures using a TRANSWELL® apparatus assay system. Assay was initiated by addition of 1×10⁻⁸ molar (¹²⁵I)-50 kHC to the upper chamber. Cultures were subsequently incubated for eighteen hours at 37° C. At the end of each experiment, contents of the basal chamber were collected and gel filtered. Void volume fractions were assayed for radioactivity and the toxin peak was summed to determine total counts. The amount of transcytosis was calculated based on the specific activity of labeled 50 kHC.

The results show that 50 kHC was poorly transported from the apical to the basolateral side of cells. 50 kHC did not efficiently cross MDCK cells, as evidenced by the rate of transcytosis of 0.06±0.00 femtomoles per hour per square centimeter. Thus, 50 kHC fragment of botulinum neurotoxin is poorly bound, internalized, transcytosed, and released by polarized MDCK epithelial cells.

### Example 25

### Apical to Basolateral Transcytosis of ALEXA FLUOR® −568 BoNT A in T-84 Cells

Transcytosis of ALEXA FLUOR® 568 BoNT A linked to ¹²⁵I at lysine residues was assayed in human gut epithelial (“T-84”) cell cultures using a TRANSWELL® apparatus assay system. Assay was initiated by addition of 1×10⁻⁸ molar ALEXA FLUOR® 568-BoNT A to the upper chamber. Cultures were subsequently incubated for eighteen hours at 37° C. At the end of each experiment contents of the basal chamber were collected and analyzed by fluorescence spectrometry. The relative amount of transcytosis was demonstrated based on the emission peak of the ALEXA FLUOR® 568 BoNT A conjugate.

The results show that purified BoNT A transported the small molecule from the apical to the basolateral side of cells. Purified BoNT A efficiently crossed T-84 cells, and the small molecule was co-transported.

There are three major conclusions that stem from the experimental results. First, the purified BoNT A is bound, internalized, transcytosed, and released by differentiated, polarized human gut epithelial cells. Second, modification of lysine residues does not alter the ability of the holotoxin to display these properties. Third, the BoNT A is capable of transporting the ALEXA FLUOR® 568 from the apical to the basolateral side of human gut epithelial cells.

### Example 26

### Apical to Basolateral Transcytosis of ALEXA FLUOR® 568 BoNT A in MDCK Cells

Transcytosis of ALEXA FLUOR® 568 BoNT A linked to ¹²⁵I at lysine residues was assayed in Madin-Darby Canine Kidney (“MDCK”) cell cultures using a TRANSWELL® apparatus assay system. Assay was initiated by addition of 1×10⁻⁸ molar ALEXA FLUOR® 568-BoNT A to the upper chamber. Cultures were subsequently incubated for eighteen hours at 37° C. At the end of each experiment contents of the basal chamber were collected and analyzed by fluorescence spectrometry. The relative amount of transcytosis was demonstrated based on the emission peak of the ALEXA FLUOR® 568-labeled BoNT A conjugate.

The results show that purified BoNT A did not efficiently transport the small molecule from the apical to the basolateral side of cells. Purified BoNT A did not efficiently co-transport the small molecule in MDCK cell cultures. The purified BoNT A is poorly bound, internalized, transcytosed, and released by differentiated, polarized canine kidney epithelial cells and therefore, incapable of transporting a small molecule across these cells.

### Example 27

### Apical to Basolateral Transcytosis of Biotin-50 kHC in T-84 Cells

Transcytosis of biotin-50 kHC, serotype A, was assayed in human gut epithelial (“T-84”) cell cultures using a TRANSWELL® apparatus assay system. Assay was initiated by addition of 1×10⁻⁷ molar biotin-50 kHC to the upper chamber. Cultures were subsequently incubated for eighteen hours at 37° C. At the end of each experiment, contents of three basal chambers per condition were collected and concentrated in a CENTRICON™ micro-concentrator. The resulting solution was run on 7.5% (w/v) SDS-PAGE and subsequently transferred to nitrocellulose membranes. The identity and molecular weight of the transcytosed molecule was confirmed by Western blotting with anti-HC antibody and by probing a duplicate blot with avidin-HRP.

The results verify that biotin-50 kHC was transported from the apical to the basolateral side of cells. Not only did biotin-50 kHC efficiently cross T-84 cells, but the molecular weight and receptor binding properties of the molecule were unaltered.

There are six conclusions that can be drawn. First, biotin-50 kHC is bound, internalized, transcytosed, and released by differentiated, polarized human gut epithelial cells. Second, modification of the fragment by addition of biotin does not alter the ability of the 50 kHC fragment to display these properties. Third, the 50 kHC fragment is capable of transporting biotin from the apical to the basolateral side of human gut epithelial cells. Fourth, the transported biotin molecule retains its ligand binding properties and associates with avidin. Fifth, the 50 kHC fragment is capable of transporting a 6×-histidine tag from the apical to the basolateral side of human gut epithelial cells. Sixth, the 50 kHC fragment is capable of transporting more than one molecule (polyhistidine tag and biotin) at a time across human gut epithelial cells.

### Example 28

### Apical to Basolateral Transcytosis of Biotin-50 kHC in MDCK Cells

Transcytosis of biotin-50 kHC, serotype A, was assayed in Madin-Darby Canine Kidney (“MDCK”) cell cultures using a TRANSWELL® apparatus assay system. Assay was initiated by addition of 1×10⁻⁷ molar biotin-50 kHC to the upper chamber. Cultures were subsequently incubated for eighteen hours at 37° C. At the end of each experiment, contents of three basal chambers per condition were collected and concentrated in a CENTRICON™ micro-concentrator. The resulting solution was run on 7.5% (w/v) SDS-PAGE and subsequently transferred to nitrocellulose membranes. The identity and molecular weight of the transcytosed molecule was confirmed by Western blotting with anti-HC antibody and by probing a duplicate blot with avidin-HRP.

The results verify that biotin-50 kHC was not efficiently transported from the apical to the basolateral side of cells. Purified 50 kHC did not efficiently co-transport 244 dalton biotin molecule in MDCK cultures.

There are two conclusions that stem from the experimental results. First, the biotin-50 kHC is not bound, internalized, transcytosed, and released by differentiated, polarized canine kidney epithelial cells. Second, the 50 kHC is not capable of transporting biotin from the apical to the basolateral side of canine kidney epithelial cells.

### Example 29

### Apical to Basolateral Transcytosis of Green Fluorescent Protein-66 kHC T-84 Cells

Transcytosis of green fluorescent protein-66 kHC, serotype A, was assayed in human gut epithelial (“T-84”) cell cultures using a TRANSWELL® apparatus assay system. Assay was initiated by addition of 1×10⁻⁸ molar GFP-66 kHC to the upper chamber. Cultures were subsequently incubated for eighteen hours at 37° C. At the end of each experiment, contents of the basal chamber were collected and analyzed by fluorescence spectrometry. The relative amount of transcytosis was demonstrated based upon the emission peak of the GFP-66 kHC conjugate.

The results show that 66 kHC transported the green fluorescent protein-66 kHC from the apical to the basolateral side of cells. Purified fragment efficiently crossed T-84 cells, and the green fluorescent protein-66 kHC was co-transported.

There are four [six ??]conclusions that can be drawn. First, 66 kHC is bound, internalized, transcytosed, and released by differentiated, polarized human gut epithelial cells. Second, modification of 66 kHC by addition of GFP does not alter the ability of 66 kHC to display these properties. Third, 66 kHC is capable of transporting GFP from the apical to the basolateral side of human gut epithelial cells. Fourth, the transported GFP molecule retains its fluorescence emitting properties. Fifth, 66 kHC is capable of transporting a 6×-histidine tag from the apical to the basolateral side of human gut epithelial cells. Sixth, 66 kHC is capable of transporting more than one molecule simultaneously (polyhistidine tag and GFP) across human gut epithelial cells.

### Example 30

### Apical to Basolateral Transcytosis of Green Fluorescent Protein-66 kHC in MDCK Cells

Transcytosis of green fluorescent protein-66 kHC was assayed in Madin-Darby Canine Kidney (“MDCK”) cell cultures using a TRANSWELL® apparatus assay system. Assay was initiated by addition of 1×10⁻⁸ molar GFP-66 kHC to the upper chamber. Cultures were subsequently incubated for eighteen hours at 37° C. At the end of each experiment, contents of the basal chamber were collected and analyzed by fluorescence spectrometry. The relative amount of transcytosis was demonstrated based upon the emission peak of the GFP-66 kHC conjugate.

The results show that 66 kHC did not efficiently transport GFP from the apical to the basolateral side of cells. Purified 66 kHC fragment did not efficiently transport the GFP in MDCK cell cultures. 66 kHC is poorly bound, internalized, transcytosed, and released by differentiated, polarized canine kidney epithelial cells.

### Example 31

### Apical to Basolateral Transcytosis of S-TAG™-50 kHC in T-84 Cells

Transcytosis of S-TAG™ 50 kHC, serotype A, was assayed in human gut epithelial (“T-84”) cell cultures using a TRANSWELL® apparatus assay system. Assay was initiated by addition of 1×10⁻⁸ molar S-TAG™-50 kHC to the upper chamber. Cultures were subsequently incubated for eighteen hours at 37° C. At the end of each experiment, contents of three basal chambers per condition were collected and concentrated in a CENTRICON™ micro-concentrator. The resulting solution was run on 7.5% (w/v) SDS-PAGE and subsequently transferred to nitrocellulose membranes. The identity and molecular weight of the transcytosed molecule was confirmed by Western blotting with anti-HC antibody.

The results verify that the S-TAG™-50 kHC was transported from the apical to the basolateral side of cells. Not only did S-TAG™-50 kHC efficiently cross T-84 cells, but the molecular weight of the molecule was unaltered.

There are six conclusions that may be drawn. First, S-TAG™-50 kHC is bound, internalized, transcytosed, and released by differentiated, polarized human gut epithelial cells. Second, modification of 50 kHC by addition of S-TAG™ does not alter the ability of 50 kHC to exhibit these properties. Third, 50 kHC is capable of transporting S-TAG™ from the apical to the basolateral side of human gut epithelial cells. Fourth, the transported S-TAG™ molecule retains its antibody binding properties. Fifth, 50 kHC is capable of transporting a 6×-histidine tag from the apical to the basolateral side of human gut epithelial cells. Sixth, 50 kHC is capable of transporting more than one molecule (polyhistidine tag and S-TAG™) at a time across human gut epithelial cells.

### Example 32

### Apical to Basolateral Transcytosis of an S-TAG™-50 kHC in MDCK Cells

Transcytosis of S-TAG™-50 kHC, serotype A, was assayed in Madin-Darby Canine Kidney (“MDCK”) cell cultures using a TRANSWELL® apparatus assay system. Assay was initiated by addition of 1×10⁻⁸ molar S-TAG™-50 kHC to the upper chamber. Cultures were subsequently incubated for eighteen hours at 37° C. At the end of each experiment, contents of three basal chambers per condition were collected and concentrated in a CENTRICON™ micro-concentrator. The resulting solution was run on 7.5% (w/v) SDS-PAGE and subsequently transferred to nitrocellulose membranes. The identity and molecular weight of the transcytosed molecule was confirmed by Western blotting with anti-HC antibody.

The results show that S-TAG™-50 kHC was not detected in medium collected from the basolateral side of cells. Thus, the S-TAG™-50 kHC did not efficiently cross MDCK cells. The S-TAG™-50 kHC is poorly bound, internalized, transcytosed, and released by polarized MDCK cells.

### Example 33

### Apical to Basolateral Transcytosis of Glutathione-S-Transferase (GST)-88 kHC Conjugate in T-84 Cells

Transcytosis of GST-88 kHC, serotype A, was assayed in human gut epithelial (“T-84”) cell cultures using a TRANSWELL® apparatus assay system. Assay was initiated by addition of 1×10⁻⁸ molar GST-88 kHC to the upper chamber. Cultures were subsequently incubated for eighteen hours at 37° C. At the end of each experiment, contents of three basal chambers per condition were collected and concentrated in a CENTRICON™ micro-concentrator. The resulting solution was run on 7.5% (w/v) SDS-PAGE and subsequently transferred to nitrocellulose membranes. The identity and molecular weight of the transcytosed molecule was confirmed by Western blotting with anti-HC antibody and by probing a duplicate blot with anti-GST antibody.

The results verify that the GST-88 kHC was transported from the apical to the basolateral side of cells. Not only did the GST-88 kHC efficiently cross T-84 cells, but the molecular weight of the molecule was unaltered.

There are six major conclusions that stem from the experimental results. The GST-88 kHC is bound, internalized, transcytosed, and released by differentiated, polarized human gut epithelial cells. Second, modification of 88 kHC by addition of GST does not alter the ability of 88 kHC to display these properties. Third, 99kHC is capable of transporting GST from the apical to the basolateral side of human gut epithelial cells. Fourth, the transported GST molecule retains its enzymatic properties. Fifth, 88 kHC is capable of transporting a 6×-histidine tag from the apical to the basolateral side of human gut epithelial cells. Sixth, 88 kHC is capable of transporting more than one molecule (polyhistidine tag and GST) at a time across human gut epithelial cells.

### Example 34

### Apical to Basolateral Transcytosis of GST-88 kHC in MDCK Cells

Transcytosis of GST-88 kHC, serotype A, was assayed in MDCK cell cultures using a TRANSWELL® apparatus assay system. Assay was initiated by addition of 1×10⁻⁸ molar GST-88 kHC to the upper chamber. Cultures were subsequently incubated for eighteen hours at 37° C. At the end of each experiment, contents of three basal chambers per condition were collected and concentrated in a CENTRICON™ micro-concentrator. The resulting solution was run on 7.5% (w/v) SDS-PAGE and subsequently transferred to nitrocellulose membranes. The identity and molecular weight of the transcytosed molecule was confirmed by Western blotting with anti-HC antibody and by probing a duplicate blot with anti-GST antibody. The results demonstrate that the GST-88 kHC was not efficiently transported from the apical to the basolateral side of cells.

### Example 35

### Apical to Basolateral Transcytosis of GST-66 kHC in T-84 Cells

Transcytosis of GST-66 kHC, serotype A, was assayed in human gut epithelial (“T-84”) cell cultures using a TRANSWELL® apparatus assay system. Assay was initiated by addition of 1×10⁻⁸ molar GST-66 kHC to the upper chamber. Cultures were subsequently incubated for eighteen hours at 37° C. At the end of each experiment, contents of three basal chambers per condition were collected and concentrated in a CENTRICON™ micro-concentrator. The resulting solution was run on 7.5% (w/v) SDS-PAGE and subsequently transferred to nitrocellulose membranes. The identity and molecular weight of the transcytosed molecule was confirmed by Western blotting with anti-HC antibody and by probing a duplicate blot with anti-GST antibody.

The results verify that GST-66 kHC was transported from the apical to the basolateral side of cells. Not only did GST-66 kHC efficiently cross T-84 cells, but the molecular weight of the molecule was unaltered.

There are six conclusions that may be drawn from the experimental results. First, GST-66 kHC is bound, internalized, transcytosed, and released by differentiated, polarized human gut epithelial cells. Second, modification of the 66 kHC by addition of GST does not alter the ability of 66 kHC to display these properties. Third, 66 kHC is capable of transporting GST from the apical to the basolateral side of human gut epithelial cells. Fourth, the transported GST molecule retains its enzymatic properties. Fifth, 66 kHC is capable of transporting a 6×-histidine tag from the apical to the basolateral side of human gut epithelial cells. Sixth, 66 kHC is capable of transporting more than one molecule (polyhistidine tag and GST) at a time across human gut epithelial cells.

### Example 36

### Apical to Basolateral Transcytosis of GST-66 kHC in MDCK Cells

Transcytosis of GST-66 kHC, serotype A, was assayed in MDCK cell cultures using a TRANSWELL® apparatus assay system. Assay was initiated by addition of 1×10⁻⁸ molar GST-66 kHC to the upper chamber. Cultures were subsequently incubated for eighteen hours at 37° C. At the end of each experiment, contents of three basal chambers per condition were collected and concentrated in a CENTRICON™ micro-concentrator. The resulting solution was run on 7.5% (w/v) SDS-PAGE and subsequently transferred to nitrocellulose membranes. The identity and molecular weight of the transcytosed molecule was confirmed by Western blotting with anti-HC antibody and by probing a duplicate blot with anti-GST antibody.

The results demonstrate that GST-66 kHC was not efficiently transported from the apical to the basolateral side of cells. Purified GST-66 kHC did not efficiently cross MDCK cultures. The GST-66 kHC is poorly bound, internalized, transcytosed, and released by polarized canine kidney epithelial cells.

### Example 37

### Apical to Basolateral Transcytosis of BoNT A by Calu-3 Cells

Transcytosis was assayed in Calu-3 cell cultures using a TRANSWELL® apparatus assay system. Assay was initiated by addition of 1×10⁻⁸ molar (¹²⁵I)-BoNT A to the upper chamber. Cultures were subsequently incubated for eighteen hours at 37° C. At each time point, the experimental contents of the basal chamber were collected and gel filtered. Void volume fractions were assayed for radioactivity and the toxin peak was summed to determine total counts. The amount of transcytosis was calculated based on the specific activity of labeled toxin.

The results show that BoNT A was transported from the apical to the basolateral side of cells. Purified neurotoxin efficiently crossed Calu-3 cells, and the rate of transcytosis was quantified at 0.423±0.076 femtomoles per hour per square centimeter.

There are five major conclusions that stem from the experimental results. First, the purified botulinum neurotoxin is bound, internalized, transcytosed, and released by differentiated, polarized human alveolar epithelial cells. Second, modification of lysine residues does not alter the ability of the holotoxin to display these properties. Third, the holotoxin is capable of transporting the ¹²⁵I-Bolton-Hunter reagent from the apical to the basolateral side of human alveolar epithelial cells. Fourth, the HC is capable of transporting the LC from the apical to the basolateral side of human alveolar epithelial cells. Fifth, the HC is capable of transporting more than one molecule (LC & Bolton-Hunter reagent) at a time across human alveolar epithelial cells.

### Example 38

### Basolateral to Apical Transcytosis of BoNT A by Calu-3 Cells

Transcytosis was assayed in Calu-3 cell cultures using a TRANSWELL® apparatus assay system. Assay was initiated by addition of 1×10⁻⁸ molar (¹²⁵I)-BoNT A to the lower chamber. Cultures were subsequently incubated for eighteen hours at 37° C. At each time point, the experimental contents of the upper chamber were collected and gel filtered. Void volume fractions were assayed for radioactivity and the toxin peak was summed to determine total counts. The amount of transcytosis was calculated based on the specific activity of labeled BoNT A.

The results show that BoNT A was transported from the basolateral to the apical side of cells. Purified neurotoxin efficiently crossed Calu-3 cells, and the rate of transcytosis was quantified at 0.206±0.037 femtomoles per hour per square centimeter.

There are five major conclusions that stem from the experimental results. First, the purified botulinum neurotoxin is bound, internalized, transcytosed, and released by differentiated, polarized human alveolar epithelial cells. This process is somewhat less efficient in the basolateral to apical direction than in the reverse direction. Second, modification of lysine residues does not alter the ability of the holotoxin to display these properties. Third, the holotoxin is capable of transporting the ¹²⁵I-Bolton-Hunter reagent from the basolateral to the apical side of human gut epithelial cells. Fourth, the HC is capable of transporting the LC from the basolateral to the apical side of human gut epithelial cells. Fifth, the HC is capable of transporting more than one molecule (LC & Bolton-Hunter reagent) at a time across human alveolar epithelial cells.

### Example 39

### Apical to Basolateral Transcytosis of A HC by Calu-3 cells

Transcytosis was assayed in Calu-3 cell cultures using a TRANSWELL® apparatus assay system. Assay was initiated by addition of 1×10⁻⁸ molar (¹²⁵I)-HC (serotype A) to the upper chamber. Cultures were subsequently incubated for eighteen hours at 37° C. At each time point, the experimental contents of the basal chamber were collected and gel filtered. Void volume fractions were assayed for radioactivity and the toxin peak was summed to determine total counts. The amount of transcytosis was calculated based on the specific activity of labeled HC.

The results show that HC was transported from the apical to the basolateral side of cells. Not only did HC efficiently cross Calu-3 cells, but the rate of transcytosis (0.198±0.007 femtomoles per hour per square centimeter) was comparable to the rate of transcytosis (0.423 femtomoles per hour per square centimeter) for purified holotoxin.

There are three major conclusions that may be drawn. First, the HC fragment of botulinum neurotoxin is bound, internalized, transcytosed, and released by differentiated, polarized human alveolar epithelial cells. Second, modification of lysine residues does not alter the ability of the HC to display these properties. Third, the HC is capable of transporting the ¹²⁵I-Bolton-Hunter reagent from the apical to the basolateral side of human alveolar epithelial cells.

### Example 40

### Basolateral to Apical Transcytosis of HC by Calu-3 cells

Transcytosis was assayed in Calu-3 cell cultures using a TRANSWELL® apparatus assay system. Assay was initiated by addition of 1×10⁻⁸ molar (¹²⁵I)-HC (serotype A) to the lower chamber. Cultures were subsequently incubated for eighteen hours at 37° C. At each time point, the experimental contents of the upper chamber were collected and gel filtered. Void volume fractions were assayed for radioactivity and the toxin peak was summed to determine total counts. The amount of transcytosis was calculated based on the specific activity of labeled HC.

The results show that HC was transported from the basolateral to the apical side of cells. Not only did HC efficiently cross Calu-3 cells, but the rate of transcytosis (0.112±0.033 femtomoles per hour per square centimeter) was comparable to the rate of transcytosis (0.206 femtomoles per hour per square centimeter) for purified holotoxin.

There are three major conclusions that may be drawn. First, the HC fragment of botulinum neurotoxin is bound, internalized, transcytosed, and released by differentiated, polarized human alveolar epithelial cells. This phenomenon operates in both the apical to basolateral and basolateral to apical directions, although the former is more efficient. Second, modification of lysine residues does not alter the ability of the HC to display these properties. Third, the HC is capable of transporting the ¹²⁵I-Bolton-Hunter reagent from the basolateral to the apical side of human alveolar epithelial cells.

### Example 41

### Apical to Basolateral Transcytosis of BoNT A by Rat Alveolar Epithelial Cells

Transcytosis was assayed in rat alveolar epithelial cells (RAEC) cultures using a TRANSWELL® apparatus assay system. Assay was initiated by addition of 1×10⁻⁸ molar (¹²⁵I-BoNT A to the upper chamber. Cultures were subsequently incubated for eighteen hours at 37° C. At each time point, the experimental contents of the basal chamber were collected and gel filtered. Void volume fractions were assayed for radioactivity and the toxin peak was summed to determine total counts. The amount of transcytosis was calculated based on the specific activity of labeled BoNT A.

The results show that BoNT A was transported from the apical to the basolateral side of cells. Purified neurotoxin efficiently crossed rat alveolar epithelial cells, and the rate of transcytosis was quantified at 0.376±0.014 femtomoles per hour per square centimeter.

There are five major conclusions that stem from the experimental results. First, the purified botulinum neurotoxin is bound, internalized, transcytosed, and released by differentiated, polarized rat alveolar epithelial cells. Second, modification of lysine residues does not alter the ability of the holotoxin to display these properties. Third, the holotoxin is capable of transporting the ¹²⁵I-Bolton-Hunter reagent from the apical to the basolateral side of rat alveolar epithelial cells. Fourth, the HC is capable of transporting the LC from the apical to the basolateral side of rat alveolar epithelial cells. Fifth, the HC is capable of transporting more than one molecule (LC & Bolton-Hunter reagent) at a time across rat alveolar epithelial cells.

### Example 42

### Basolateral to Apical Transcytosis of BoNT A by RAEC

Transcytosis was assayed in rat alveolar epithelial cell cultures using a TRANSWELL® apparatus assay system. Assay was initiated by addition of 1×10⁻⁸ molar (¹²⁵I)-BoNT A to the lower chamber. Cultures were subsequently incubated for eighteen hours at 37° C. At each time point, the experimental contents of the upper chamber were collected and gel filtered. Void volume fractions were assayed for radioactivity and the toxin peak was summed to determine total counts. The amount of transcytosis was calculated based on the specific activity of labeled BoNT A.

The results show that BoNT A was transported from the basolateral to the apical side of cells. Purified neurotoxin efficiently crossed rat alveolar epithelial cells, and the rate of transcytosis was quantified at 0.159±0.027 femtomoles per hour per square centimeter.

There are five major conclusions that stem from the experimental results. First, the purified botulinum neurotoxin is bound, internalized, transcytosed, and released by differentiated, polarized rat alveolar epithelial cells. This phenomenon operates in both the apical to basolateral and basolateral to apical directions, although the former is more efficient. Second, modification of lysine residues does not alter the ability of the holotoxin to display these properties. Third, the holotoxin is capable of transporting the ¹²⁵I-Bolton-Hunter reagent from the basolateral to the apical side of rat alveolar epithelial cells. Fourth, the HC is capable of transporting the LC from the basolateral to the apical side of rat alveolar epithelial cells. Fifth, the HC is capable of transporting more than one molecule (LC & Bolton-Hunter reagent) at a time across rat alveolar epithelial cells.

### Example 43

### Apical to Basolateral Transcytosis of HC by RAEC

Transcytosis was assayed in rat alveolar epithelial cells (RAEC) using a TRANSWELL® apparatus assay system. Assay was initiated by addition of 1×10⁻⁸ molar (¹²⁵I)-HC (serotype A) to the upper chamber. Cultures were subsequently incubated for 18 hours at 37° C. At each time point, the experimental contents of the basal chamber were collected and gel filtered. Void volume fractions were assayed for radioactivity and the toxin peak was summed to determine total counts. The amount of transcytosis was calculated based on the specific activity of labeled HC.

The results show that HC was transported from the apical to the basolateral side of cells. Not only did HC efficiently cross rat alveolar epithelial cells, but the rate of transcytosis (0.140±0.050 femtomoles per hour per square centimeter) was comparable to the rate of transcytosis (0.376 femtomoles per hour per square centimeter) for botulinum neurotoxin type A.

There are three major conclusions that may be drawn. First, the HC fragment of botulinum neurotoxin is bound, internalized, transcytosed, and released by differentiated, polarized rat alveolar epithelial cells. Second, modification of lysine residues does not alter the ability of the HC to display these properties. Third, the HC is capable of transporting the ¹²⁵I-Bolton-Hunter reagent from the apical to the basolateral side of human alveolar epithelial cells.

### Example 44

### Basolateral to Apical Transcytosis of HC by RAEC

Transcytosis was assayed in rat alveolar epithelial cells using a TRANSWELL® apparatus assay system. Assay was initiated by addition of 1×10⁻⁸ molar (¹²⁵I)-HC (serotype A) to the lower chamber. Cultures were subsequently incubated for eighteen hours at 37° C. At each time point, the experimental contents of the upper chamber were collected and gel filtered. Void volume fractions were assayed for radioactivity and the toxin peak was summed to determine total counts. The amount of transcytosis was calculated based on the specific activity of labeled HC.

The results show that HC was transported from the basolateral to the apical side of cells. Not only did HC efficiently cross rat alveolar epithelial cells, but the rate of transcytosis (0.132±0.026 femtomoles per hour per square centimeter) was comparable to the rate of transcytosis (0.159 femtomoles per hour per square centimeter) for botulinum neurotoxin type A.

There are three major conclusions that may be drawn. First, the HC fragment of botulinum neurotoxin is bound, internalized, transcytosed, and released by differentiated, polarized rat alveolar epithelial cells. This phenomenon operates in both the apical to basolateral and Basolateral to apical directions, although the former is more efficient. Second, modification of lysine residues does not alter the ability of the HC to display these properties. Third, the HC is capable of transporting the ¹²⁵I-Bolton-Hunter reagent from the basolateral to the apical side of rat alveolar epithelial cells.

### Example 45

### Absorption of BoNT A from the Respiratory Tract of Mouse

This example demonstrated that botulinum toxin contains all information necessary to bind receptors on the apical surface of epithelial cells, to be internalized, to be transcytosed, and to be released on the basolateral side of epithelial cells in living animals. These experiments were done with homogeneous BoNT and with Swiss-Webster female mice (25 to 30 grams body weight).

BoNT A was administered by the intranasal route. After mice were lightly anesthetized with isoflurane (ISO-THESIA™, Abbott Laboratories North, Chicago, Ill.), 36.4 micrograms per kilogram weight (¹²⁵I)-BoNT A was administered by a single application of a 20 microliter solution to the nares. The heads of animals were maintained in an upright position to minimize drainage into the posterior pharynx. Individual groups were sacrificed at 1, 2, or 3 hours with CO₂, and blood was collected by cardiac puncture. Plasma was separated from blood by centrifugation at 3000×g for 10 minutes and then stored at −20° C. until assay. Plasma samples (100 microliters) were subsequently mixed with PBS (400 microliters) and filtered through a SEPHADEX™ G-25 column. Fractions (0.5 milliliter) were collected, and the amount of radioactivity in the fractions was measured in a gamma-counter. Labeled BoNT A eluted at void volume, and the radioactivity contained in the void volume fractions was summed to determine the total amount of protein present.

The results shown in FIG. 23 indicate that the botulinum toxin was absorbed from the respiratory tract. The timepoint for maximum protein concentration in blood was approximately two hours, and there was rapid clearance after attainment of the peak values.

There are five major conclusions that stem from the experimental results. First, the purified botulinum neurotoxin is bound, internalized, transcytosed, and released by respiratory tract epithelial cells in vivo. Second, modification of lysine residues does not alter the ability of the holotoxin to display these properties. Third, the holotoxin is capable of transporting the ¹²⁵I-Bolton-Hunter reagent from the apical to the basolateral side of mouse respiratory epithelial cells in vivo. Fourth, the HC is capable of transporting the LC from the apical to the basolateral side of mouse respiratory epithelial cells in vivo. Fifth, the HC is capable of transporting more than one molecule (LC & Bolton-Hunter reagent) at a time across mouse respiratory epithelial cells in vivo.

### Example 46

### Absorption of native HC from the Respiratory Tract of Mouse

This example demonstrated that HC (serotype A) contains all information necessary to bind receptors on the apical surface of epithelial cells, to be internalized, to be transcytosed, and to be released on the basolateral side of epithelial cells in living animals. These experiments were done with homogeneously isolated HC of BoNT and with Swiss-Webster female mice (25 to 30 grams body weight).

HC was administered by the intranasal route. After mice were lightly anesthetized with isoflurane (ISO-THESIA™, Abbott Laboratories North, Chicago, Ill., U.S.A.), 24.4 micrograms per kilogram body weight (¹²⁵I)-HC was administered by a single application of a 20 microliter solution to the nares. The heads of animals were maintained in an upright position to minimize drainage into the posterior pharynx. Individual groups were sacrificed at one, two, four or six hours with CO₂, and blood was collected by cardiac puncture. Plasma was separated from blood by centrifugation at 3000×g for 10 minutes and then stored at −20° C. until assay. Plasma samples (100 microliters) were subsequently mixed with PBS (400 microliters) and filtered through a SEPHADEX™ G-25 column. Fractions (0.5 milliliter) were collected, and the amount of radioactivity in the fractions was measured in a gamma-counter. Labeled HC eluted at void volume, and the radioactivity contained in the void volume fractions was summed to determine the total amount of HC present.

Animals that received HC were monitored for 6 hours. The results shown in FIG. 24 indicate that HC was absorbed from the respiratory tract. The timepoint for maximum protein concentration in blood was approximately two hours, and there was rapid clearance after attainment of the peak values.

There are three major conclusions that may be drawn. First, the HC fragment of botulinum neurotoxin is bound, internalized, transcytosed, and released by respiratory tract epithelial cells in vivo. Second, modification of lysine residues does not alter the ability of the HC to display these properties. Third, the HC is capable of transporting the ¹²⁵I-Bolton-Hunter reagent from the apical to the basolateral side of mouse respiratory epithelial cells in vivo.

### Example 47

### Absorption of recombinant 50 kHC from the Respiratory Tract of Mouse

This example demonstrated that 50 kHC contains all information necessary to bind receptors on the apical surface of epithelial cells, to be internalized, to be transcytosed, and to be released on the basolateral side of epithelial cells in living animals. These experiments were done with purified recombinant 50 kHC fused with a 6× His tag and with Swiss-Webster female mice (body weight of 25 to 30 grams each).

50 kHC was administered by the intranasal route. After mice were lightly anesthetized with isoflurane (ISO-THESIA™, Abbott Laboratories North, Chicago, Ill., U.S.A.), 0.4 milligrams of protein per kilogram body weight was administered by a single application of a 20 microliter solution to the nares. The heads of animals were maintained in an upright position to minimize drainage into the posterior pharynx. Individual groups were sacrificed at 0.5, 1, 2, or 4 hours with CO₂ and blood was collected by cardiac puncture. Plasma was separated from blood by centrifugation at 3000× g for 10 minutes and then stored at −20° C. until assay. Plasma level of the HC fragment was determined by capture ELISA.

Animals that received 50 kHC were monitored for four hours. The results shown in FIG. 25 that 50 kHC was absorbed from the respiratory tract. The timepoint for maximum protein concentration in blood was approximately one hour, and there was rapid clearance after attainment of peak values.

There are two major conclusions that stem from the experimental results. First, the 50 kHC fragment of botulinum neurotoxin is bound, internalized, transcytosed, and released by mouse respiratory tract epithelial cells in vivo. Second, 50 kHC is capable of transporting a 6×-histidine tag from the apical to the basolateral side of mouse respiratory tract epithelial cells in vivo.

### Example 48

### Absorption of GST-50 kHC from the Respiratory Tract of Mouse

This example demonstrated that 50 kHC contains all information necessary to bind receptors on the apical surface of epithelial cells, to be internalized, to be transcytosed, and to be released on the basolateral side of epithelial cells in living animals. In addition, 50 kHC can transport GST across epithelial cells. These experiments were done with purified recombinant GST-50 kHC and Swiss-Webster female mice (body weight of 25 to 30 grams each).

GST-50 kHC fusion protein was administered by the intranasal route. After mice were lightly anesthetized with isoflurane (ISO-THESIA™, Abbott Laboratories North, Chicago, Ill., U.S.A.), 0.6 micrograms per kilogram body weight protein was administered by a single application of a 20 microliter solution to the nares. The heads of animals were maintained in an upright position to minimize drainage into the posterior pharynx. Individual groups were sacrificed at one, two, four or six hours with CO₂, and blood was collected by cardiac puncture. Plasma was separated from blood by centrifugation at 3000× g for 10 minutes and then stored at −20° C. until assay. Plasma level of GST-50 kHC was determined by capture ELISA.

Animals that received GST-50 kHC were monitored for six hours. The results shown in FIG. 26 indicate that GST-50 kHC was absorbed from the respiratory tract. The timepoint for maximum protein concentration in blood was approximately two hours, and there was rapid clearance after attainment of the peak values.

There are four major conclusions that may be drawn from the experimental results. First, the GST-50 kHC is bound, internalized, transcytosed, and released by mouse respiratory tract epithelial cells in vivo. Second, 50 kHC is capable of transporting GST from the apical to the basolateral side of mouse respiratory tract epithelial cells in vivo. Third, 50 kHC is capable of transporting a 6×-histidine tag from the apical to the basolateral side of mouse respiratory tract epithelial cells in vivo. Fourth, 50 kHC is capable of transporting more than one molecule (GST & 6×-histidine tag) at a time across mouse respiratory epithelial cells in vivo.

### Example 49

### Intranasal Immunization of Mice with GST-50 kHC

This example demonstrated that 50 kHC contains all information necessary to bind receptors on the apical surface of epithelial cells, to be internalized, to be transcytosed, and to be released on the Basolateral side of epithelial cells in living animals. In addition, 50 kHC can transport a heterologous molecule in the correct conformation to evoke an immune response. These experiments were done with purified recombinant GST-50 kHC and Swiss-Webster female mice (body weight of 25-30 grams each).

For intranasal immunization, mice received 0.6 milligrams of GST-50 kHC per kilogram body weight in 20 microliters of PBS. Mice were lightly anesthetized with isoflurane (ISO-THESIA™, Abbott Laboratories North, Chicago, Ill., U.S.A.). Protein was administered by a single application of a 20 microliter solution to the nares. The heads of animals were maintained in an upright position to minimize drainage into the posterior pharynx. Five doses were given at seven day intervals. The mice were bled 10 days after the fifth immunization, and the specimens were analyzed by immunoblotting for immunoreactivity to GST, 50 kHC, and GST-50 kHC.

Animals that are immunized with GST-50 kHC were monitored for the presence of specific antibodies. The results shown in the Western blot of immunized mouse serum of FIG. 27 show that these animals developed antibodies against both GST and BoNT A HC following intranasal immunization with GST-50 kHC. These results indicate that 50 kHC carries GST molecule from the respiratory tract to the blood stream in vivo, and that specific immune responses to GST and 50 kHC were evoked by intranasal immunization.

There are five major conclusions that may be drawn from the experimental results. First, the GST-50 kHC is bound, internalized, transcytosed, and released by mouse respiratory tract epithelial cells in vivo. Second, 50 kHC is capable of transporting GST from the apical to the basolateral side of mouse respiratory tract epithelial cells in vivo. Third, the 50 kHC is capable of transporting a 6×-histidine tag from the apical to the basolateral side of mouse respiratory tract epithelial cells in vivo. Fourth, the 50 kHC is capable of transporting more than one molecule (GST & 6×-histidine tag) at a time across mouse respiratory epithelial cells in vivo. Fifth, transcytosed GST-50 kHC is capable of evoking specific immune response to GST and 50 kHC in vivo.

### Example 50

### Intranasal Immunization of Mice With BoNT A 48 kilodalton HC Portion

This example demonstrates that a two kilodalton segment of the HC carboxyterminus contains information necessary to bind receptors on the apical surface of epithelial cells, to be internalized, to be transcytosed, and to be released on the basolateral side of the epithelial cells in living animals. To demonstrate this, a 48 kilodalton portion of the HC was generated by deleting a 2 kilodalton fragment of carboxyterminus end of 50 kHC with trypsin digestion.

The experiment was carried out using purified recombinant 48 kHC and Swiss-Webster female mice (25 to 30 gram body weight).

For intranasal immunization, mice received 0.4 mg/kg of 48 kHC in a 10 μl of PBS. Mice were lightly anesthetized with isoflurane (ISO-THESIA™, Abbott Laboratories North, Chicago, Ill., U.S.A.). Protein was administered by a single application of 10 μl solution to the nares. The heads of animals were maintained in an upright position to minimize drainage into the posterior pharynx. Three doses were given at two week intervals. The mice were bled seven days after the third immunization, and the specimens were analyzed by ELISA for immunoreactivity to botulinum toxin A. The mice were also challenged with a lethal dose of botulinum toxin A (1 μg/mouse) ten days after the third immunization, and the survival rate was observed for two weeks.

All the animals died within twenty four hours after the lethal challenge of botulinum toxin (i.e., there was little protection). In addition, there was only a low level of serum IgG to the antigen. The results show that the deleted 2 kilodalton fragment of the carboxyterminus end of the HC has an important function in binding, internalization, transcytosis, and release by mouse respiratory epithelial cells in vivo.

### Example 51

### Intranasal Immunization of Mice with 50 kHC and Cholera Toxin B Subunit

This example demonstrates that cholera toxin B subunit (CTB) induces systemic immune response against BoNT as well as mucosal immune response when coadministered with 50 kHC by intranasal route. These experiments were done with purified recombinant 50 kHC, cholera toxin B subunit (sigma) and Swiss-Webster female mice (body weight of 25-30 grams each).

Mice received 0.1 milligrams of CTB per kilogram body weight in 10 microliters of PBS, 40 micrograms of 50 kHC per kilogram body weight in 10 microliters of PBS, or 50 kHC and CTB by intranasal route. Mice were lightly anesthetized with isoflurane (ISO-THESIA™, Abbott Laboratories North, Chicago, Ill., U.S.A.). Protein was administered by a single application of 10 microliters of solution to the nares. The heads of animals were maintained in an upright position to minimize drainage into the posterior pharynx. Three doses were given at two week intervals. The mice were bled seven days after the third immunization, and the specimens were analyzed by ELISA for immunoreactivity to botulinum toxin A. The mice were also challenged with lethal dose of botulinum toxin A (1 μg/mouse) ten days after the third immunization, and the survival rate of observed for two weeks.

The animals immunized with CTB were dead within 2 hours after the lethal challenge of botulinum toxin. The animals immunized with low dose of 50 kHC showed 40% of protection against the BoNT A challenge and moderate level of serum Ig response (FIG. 28). The animals immunized with 50 kHC and CTB developed significant high level of serum Ig response as well as mucosal IgG response, and all the animals were protected against the lethal challenge of BoNT A.

These results showed that intranasal administration of 50 kHC does not induce a mucosal immune response even at the high dose of 0.4 milligram per kilogram, although it induces a modest level of serum Ig response and protects only 40% of animals against the lethal challenge of BoNT A. However, mucosal IgG response can be induced by co-administration with CTB, which leads to complete protection against a multilethal dose of toxin (1 μg/mouse).

### Example 52

### Oral Immunization of Mice with Purified Serotype A HC (100 kDa HC)

This example demonstrates that 100 kDa HC contains all the information necessary to bind receptors on the apical surface of intestinal cells, to be internalized, to be transcytosed, and to be released on the basolateral side of epithelial cells in living animals. These experiments were performed using HC purified from native botulinum neurotoxin serotype A, administered by gavage to Swiss-Webster female mice (body weight 25-30 grams).

For oral immunization, mice received 10 micrograms of 100 kDa HC per mouse in a 200 microliters of PBS. HC in 200 microliters of PBS was administered to each mouse using a feeding needle. An initial dose, was followed by three boosters at two week intervals. Mice were anesthethized with isoflurane (ISO-THESIA™, Abbott Laboratories, Chicago, Ill., U.S.A.), and bled from the retroorbital sinus seven days after each booster. Antibody production was assayed by ELISA and titers were calculated.

Antibody titers subsequent to each booster are illustrated in FIG. 29. The results demonstrate that mice immunized orally with 100 kDa HC developed antibodies to toxin HC. The results indicate that 100 kDa HC was absorbed from the gastrointestinal system to the circulation in vivo, and that a specific immune response was evoked by oral immunization with 100 kDa HC. Furthermore, the results show that purified 100 kDa HC is an oral vaccine against botulinum neurotoxin.

### Example 53

### Transport of Various Molecules of Differing Molecular Sizes and Differing Molecular Functions across Epithelial Cells In Vitro and In Vivo

This example demonstrates that the HC of botulinum toxin, as well as fragments of the HC, have a very broad capacity to transport molecules across epithelial cells. This means that the transport molecule (i.e., the HC or fragments of the HC) can carry a wide array of cargo molecules (ligands, enzymes, antigens, etc.) into the general circulation by binding to the surface of epithelial cells, undergoing endocytosis and transcytosis, and then release into blood and lymph.

In vitro experiments and in vivo experiments were performed as described in Examples 1 to 52. The results of these experiments demonstrate that the HC or its fragments can transport molecules of widely differing molecular weights, as shown in Table 2, below, and widely differing functions, as shown in Table 2, below, across epithelial cells.

TABLE 2
Molecules of varying sizes

| Molecule | Size (kilodaltons) |
|---|---|
| biotin | 244 |
| Bolton-Hunter Reagent (¹²⁵I) | 387 |
| Alexa 568 | 792 |
| 6 × histidine tag | 840 |
| S-Tag | 1,748 |
| glutathione-S-transferase | 26,000 |
| (GST) | |
| Green Fluorescent protein | 27,000 |
| (GFP) | |
| BoNT LC | 50,000 |

TABLE 3
Molecules of Differing Functions

| Molecule | Functional Properties |
|---|---|
| biotin | ligand binding |
| horseradish peroxidase | exhibits catalytic activity |
| Bolton-Hunter Reagent (¹²⁵I) | emits ionizing radiation |
| Alexa 568 | emits fluorescent signal |
| various antigens | evoke antibody response |
| BoNT LC | exhibits catalytic activity |

There are eight major conclusions that stem from the experimental results. First, the HC of botulinum toxin is bound, internalized, transcytosed and released by epithelial cells that form a boundary between the outside world and the general circulation. Second, fragments of the HC of botulinum toxin are bound, internalized, transcytosed and released by epithelial cells that form a boundary between the outside world and the general circulation. Third, the HC and its fragments, when modified to allow for attachment of individual molecules, continue to display all the properties needed to cross epithelial barriers. Fourth, the HC and its fragments can transport a variety of molecules individually across epithelial cells. Fifth, when modified to allow for attachment of more than one molecule, the HC and its fragments continue to display all the properties needed to cross epithelial cells. Sixth, the HC and its fragments can transport more than one molecule at a time across epithelial cells. Seventh, the HC and its fragments can transport molecules of differing molecular weights at the same time. Eighth, the HC and its fragments can transport molecules of differing functions at the same time.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A composition for translocating an entity across a non-keratinized epithelium of an animal, the composition comprising an entity linked to a carboxyterminal fragment of the HC of a Clostridium botulinum neurotoxin (BoNT), wherein the size of the entity is not greater than the lumenal capacity of vesicles of cells of the epithelium.

2. The composition of claim 1, wherein the BoNT is selected from the group consisting of the BoNTs of the serotypes A, B, and E.

3. The composition of claim 1, wherein the fragment comprises at least about 2% by molecular mass of the HC of a Clostridium botulinum neurotoxin (BoNT).

4. The composition of claim 1, wherein the fragment comprises at least about 5% by molecular mass of the HC of a Clostridium botulinum neurotoxin (BoNT).

5. The composition of claim 1, wherein the fragment comprises at least about 30% by molecular mass of the HC of a Clostridium botulinum neurotoxin (BoNT).

6. The composition of claim 1, wherein the fragment comprises at least about 50% by molecular mass of the HC of a Clostridium botulinum neurotoxin (BoNT).

7. The composition of claim 1, wherein the fragment comprises about 20 to about 50 residues of the HC of a Clostridium botulinum neurotoxin (BoNT).

8. The composition of claim 1, wherein the fragment comprises at least about 35 amino acid residues of the HC of a Clostridium botulinum neurotoxin (BoNT).

9. The composition of claim 1, wherein the fragment comprises at least about 60 amino acid residues of the HC of a Clostridium botulinum neurotoxin.

10. The composition of claim 1, wherein the fragment comprises a domain selected from a β-trefoil domain and a lectin binding domain.

11. The composition of claim 1, wherein the fragment is linked to the entity by an intervening molecule.

12. The composition of claim 11, wherein the intervening molecule is selected from the group consisting of avidin, an antibody substance, and biotin.

13. The composition of claim 1, wherein the entity is linked to the fragment by a peptide bond.

14. The composition of claim 1, wherein the entity is linked near the amino terminal end of the fragment.

15. The composition of claim 1, wherein the entity is linked to the amino terminal end of the fragment.

16. The composition of claim 1, wherein the entity is a supramolecular complex.

17. The composition of claim 16, wherein the supramolecular complex is a multi-subunit protein, at least one sub-unit of the protein being linked to the fragment.

18. The composition of claim 1, wherein the entity is a polypeptide.

19. The composition of claim 18, wherein the polypeptide is an immunogenic portion of a protein associated with a pathogen of an animal.

20. The composition of claim 19, wherein the pathogen is selected from the group consisting of Bacillus anthracis, Bordetella pertussis, Brucella abortus, Brucella canis, Brucella melitensis, Brucella suis, Clostridium perfringens, Clostridium tetani, Corynebacterium diptheriae, Coxiella burnetii, Crimean-Congo hemorrhagic fever virus, Francisella tularensis, Pseudomonas pseudomallei, ricin, Rift Valley fever virus, the coronavirus that is the causative agent of Sudden Acute Respiratory Syndrome (SARS), saxitoxin, Staphylococcal enterotoxin B, trichothecene mycotoxins, Variola major Venezuelan equine encephalitis viruses, and Vibrio cholera.

21. The composition of claim 19, wherein the animal is a human and the pathogen is Clostridium botulinum neurotoxin.

22. The composition of claim 1, wherein the entity is an antibody substance.

23. The composition of claim 22, wherein the antibody substance is selected from the group consisting of a tetra-subunit immunoglobulin and a single-chain antibody.

24. The composition of claim 1, further comprising a plurality of entities.

25. The composition of claim 1, wherein the animal is a mammal.

26. The composition of claim 1, wherein the epithelium is selected from the group consisting of anal epithelium, gastrointestinal epithelium, nasal epithelium, ocular epithelium, pulmonary epithelium, and vaginal epithelium.

27. The composition of claim 1, wherein the molecular mass of the entity is no greater than about 1000 daltons.

28. The composition of claim 1, wherein the molecular mass of the entity is about 300 daltons to about 550 daltons.

29. The composition of claim 1, further comprising an auxiliary protein selected from the group consisting of polypeptides of SEQ ID NOs: 20 to 168, and 170 to 188.

30. A composition that elicits an immune response against an antigen in a vertebrate, the composition comprising at least one epitope of the antigen linked to a carboxyterminal fragment of the HC of a Clostridium botulinum neurotoxin (BoNT), wherein the size of the epitope is not greater than the lumenal capacity of vesicles of cells of the epithelium.

31. The composition of claim 30, wherein the immune response is a systemic immune response.

32. The composition of claim 30, wherein the immune response is a mucosal immune response.

33. The composition of claim 30, wherein the antigen is selected from antigens of Bacillus anthracis, Bordetella pertussis, Brucella abortus, Brucella canis, Brucella melitensis, Brucella suis, Clostridium perfringens, Clostridium tetani, Corynebacterium diptheriae, Coxiella burnetii, Crimean-Congo hemorrhagic fever virus, Francisella tularensis, Pseudomonas pseudomallei, ricin, Rift Valley fever virus, the coronavirus that is the causative agent of Sudden Acute Respiratory Syndrome (SARS), saxitoxin, smallpox virus, Staphylococcal enterotoxin B, trichothecene mycotoxins, Variola major Venezuelan equine encephalitis viruses, and Vibrio cholera.

34. The composition of claim 30, wherein the fragment comprises at least about 2% by molecular mass of the HC of a Clostridium botulinum neurotoxin (BoNT).

35. The composition of claim 30, wherein the fragment comprises at least about 5% by molecular mass of the HC of a Clostridium botulinum neurotoxin (BoNT).

36. The composition of claim 30, wherein the fragment comprises at least about 30% by molecular mass of the HC of a Clostridium botulinum neurotoxin (BoNT).

37. The composition of claim 30, wherein the fragment comprises at least about 50% by molecular mass of the HC of a Clostridium botulinum neurotoxin (BoNT).

38. The composition of claim 30, wherein the fragment comprises about 20 to about 50 residues of the HC of a Clostridium botulinum neurotoxin (BoNT).

39. The composition of claim 30, wherein the fragment comprises at least about 35 amino acid residues of the HC of a Clostridium botulinum neurotoxin (BoNT).

40. The composition of claim 30, wherein the fragment comprises at least about 60 amino acid residues of the HC of a Clostridium botulinum neurotoxin.

41. A composition that elicits an immune response against Botulinum neurotoxin in a vertebrate, the composition comprising a carboxyterminal fragment of the HC of a Clostridium botulinum neurotoxin (BoNT).

42. The composition of claim 41, wherein the immune response is a systemic immune response.

43. The composition of claim 41, wherein the immune response is a mucosal immune response.

44. The composition of claim 41, wherein the fragment comprises at least about 2% by molecular mass of the HC of a Clostridium botulinum neurotoxin (BoNT).

45. The composition of claim 41, wherein the fragment comprises at least about 5% by molecular mass of the HC of a Clostridium botulinum neurotoxin (BoNT).

46. The composition of claim 41, wherein the fragment comprises at least about 30% by molecular mass of the HC of a Clostridium botulinum neurotoxin (BoNT).

47. The composition of claim 41, wherein the fragment comprises at least about 50% by molecular mass of the HC of a Clostridium botulinum neurotoxin (BoNT).

48. A vaccine comprising an antigen linked to a carboxyterminal fragment of HC of a Clostridium botulinum neurotoxin (BoNT), wherein the antigen induces protective immunity against a pathogen of a vertebrate when the antigen is delivered to the circulation of the vertebrate.

49. The vaccine of claim 48, formulated for administration to a human by a route selected from the group consisting of anal, nasal, pulmonary, ocular, oral and vaginal routes.

50. The vaccine of claim 48, comprising a plurality of antigens that induce immunity against a plurality of pathogens, wherein each antigen is linked to a fragment of a HC of Clostridium botulinum neurotoxin (BoNT).

51. A vaccine comprising an antigen linked to a carboxyterminal fragment of a HC of a Clostridium botulinum neurotoxin (BoNT), wherein the antigen induces protective immunity against Clostridium botulinum neurotoxin in a vertebrate when the antigen is delivered to the circulation of the vertebrate.

52. A composition that elicits an immune response against an antigen when the antigen is contacted with a non-keratinized epithelium of a vertebrate, the composition comprising at least one epitope of the antigen linked to a carboxyterminal fragment of the HC a Clostridium botulinum neurotoxin (BoNT).

53. A method of translocating an entity across a non-keratinized epithelium of an animal, wherein the size of the entity is not greater than the lumenal capacity of vesicles of cells of the epithelium, the method comprising contacting the epithelium with a composition comprising the entity linked to a carboxyterminal fragment of the HC of a Clostridium botulinum neurotoxin (BoNT).

54. The method of claim 53, wherein the BoNT is selected from the group consisting of the BoNTs of the serotypes A, B, and E.

55. The method of claim 53, wherein the fragment comprises at least about 2% by molecular mass of the HC of a Clostridium botulinum neurotoxin (BoNT).

56. The method of claim 53, wherein the fragment comprises at least about 5% by molecular mass of the HC of a Clostridium botulinum neurotoxin (BoNT).

57. The method of claim 53, wherein the fragment comprises at least about 30% by molecular mass of the HC of a Clostridium botulinum neurotoxin (BoNT).

58. The method of claim 53, wherein the fragment comprises at least about 50% by molecular mass of the HC of a Clostridium botulinum neurotoxin (BoNT).

59. The method of claim 53, wherein the fragment comprises about 20 to about 50 residues of the HC of a Clostridium botulinum neurotoxin (BoNT).

60. The method of claim 53, wherein the fragment comprises at least about 35 amino acid residues of the HC of a Clostridium botulinum neurotoxin (BoNT).

61. The method of claim 53, wherein the fragment comprises at least about 60 amino acid residues of the HC of a Clostridium botulinum neurotoxin.

62. The method of claim 53, wherein the fragment is linked to the entity by an intervening molecule.

63. The method of claim 61, wherein the intervening molecule is selected from the group consisting of avidin, an antibody substance, and biotin.

64. The method of claim 53, wherein the entity is linked to the fragment by a peptide bond.

65. The method of claim 53, wherein the entity is linked near the amino terminal end of the fragment.

66. The method of claim 53, wherein the entity is linked to the amino terminal end of the fragment.

67. The method of claim 53, wherein the entity is a supramolecular complex.

68. The method of claim 53, wherein the supramolecular complex is a multi-subunit protein, at least one sub-unit of the protein being linked to the fragment.

69. The method of claim 53, wherein the entity is a polypeptide.

70. The method of claim 69, wherein the polypeptide is an immunogenic portion of a protein associated with a pathogen of an animal.

71. The method of claim 69, wherein the pathogen is selected from the group consisting of Bacillus anthracis, Bordetella pertussis, Brucella abortus, Brucella canis, Brucella melitensis, Brucella suis, Clostridium perfringens, Clostridium tetani, Corynebacterium diptheriae, Coxiella burnetii, Crimean-Congo hemorrhagic fever virus, Francisella tularensis, Pseudomonas pseudomallei, ricin, Rift Valley fever virus, the coronavirus that is the causative agent of Sudden Acute Respiratory Syndrome (SARS), saxitoxin, smallpox virus, Staphylococcal enterotoxin B, trichothecene mycotoxins, Variola major, Venezuelan equine encephalitis viruses, and Vibrio cholera.

72. The method of claim 69, wherein the animal is a human and the pathogen is Clostridium botulinum neurotoxin.

73. The method of claim 53, wherein the entity is an antibody substance.

74. The method of claim 53, wherein the antibody substance is selected from the group consisting of a tetra-subunit immunoglobulin and a single-chain antibody.

75. The method of claim 53 further comprising a plurality of entities.

76. The method of claim 53 wherein the animal is a mammal.

77. The method of claim 54, wherein the composition further comprises an auxiliary protein selected from the group consisting of the polypeptides SEQ ID NOs: 20 to 168, and 170 to 188.

78. A method of inducing an immune response against an entity in a vertebrate, the method comprising
a) linking the entity to a carboxyterminal fragment of the HC of a Clostridium botulinum neurotoxin (BoNT), wherein the size of the entity is not greater than the lumenal capacity of vesicle cells of the epithelium; and
b) contacting the fragment-linked entity with the epithelium.

79. The method of claim 78, wherein the vertebrate is a human.

80. The method of claim 79, wherein the entity is an antigen of a human pathogen.

81. A method of inducing an immune response against Clostridium neurotoxin (BoNT) in a vertebrate, the method comprising contacting a composition to an epithelium of a vertebrate, wherein the composition comprises an entity linked to a carboxyterminal fragment of HC of a Clostridium botulinum neurotoxin (BoNT) and the entity is an antigen that induces protective immunity against botulinum neurotoxin in a vertebrate when the antigen is delivered to the circulation of the vertebrate.

82. A pharmaceutical composition for rapid delivery of an entity to the bloodstream of a vertebrate, the composition comprising the entity linked to a carboxyterminal fragment of the HC of Clostridium botulinum neurotoxin (BoNT), wherein the composition is formulated for pulmonary administration.

83. A pharmaceutical composition for rapid delivery of an entity to the bloodstream of a vertebrate, the composition comprising the entity linked to a carboxyterminal fragment of the HC of Clostridium botulinum neurotoxin (BoNT), wherein the composition is formulated for oral administration.

84. A foodstuff, wherein the genome of an ingredient of the foodstuff comprises a polynucleotide expressibly encoding a protein that comprises a therapeutic or an immunogenic polypeptide linked to a carboxyterminal fragment of the HC of a Clostridium botulinum neurotoxin.

85. A translocating polypeptide that comprises an immunogenic polypeptide linked to a carboxyterminal fragment of the HC of a Clostridium botulinum neurotoxin.
